(19) United States
(12) Patent Application Publication
(10) Pub. No.: US 2024/0331863 A1
(43) Pub. Date: Oct. 3, 2024

(54) BIOMARKERS AND RELATED METHODS FOR DETECTING INFLAMMATORY BOWEL DISEASE AND DISCRIMINATING BETWEEN CROHN'S DISEASE AND ULCERATIVE COLITIS

(71) Applicant: PRECOGIFY PHARMACEUTICAL CHINA CO., LTD., Beijing (CN)

(72) Inventors: Kai LIN, Beijing (CN); Xudong DAI, Beijing (CN); Yu TIAN, Beijing (CN); Xiaowei LI, Beijing (CN); Tengsong CUI, Beijing (CN); Mingyuan MA, Beijing (CN)

(73) Assignee: PRECOGIFY PHARMACEUTICAL CHINA CO., LTD., Beijing (CN)

(21) Appl. No.: 18/677,942

(22) Filed: May 30, 2024

Related U.S. Application Data
(63) Continuation of application No. PCT/CN2023/073422, filed on Jan. 20, 2023.

(51) Int. Cl. G16H 50/20; G16H 10/60

(52) U.S. Cl. CPC G16H 50/20; G16H 10/60

(57) ABSTRACT

The present disclosure provides panels of metabolites for use as diagnostic biomarkers for detecting IBD in a subject and panels of metabolites for use as diagnostic biomarkers for discriminating between UC and CD, which are the main subtypes of IBD. Panels of metabolites for used as diagnostic biomarkers for discriminating between IBD and colorectal polyp are also provided. These diagnostic biomarkers are serum metabolites associated with gut microbiome. Systems and methods for detecting IBD and for discriminating between UC and CD using the panels of metabolites are also provided.

### CROSS REFERENCE TO RELATED APPLICATION

The present application is a Continuation of International application No. PCT/CN2023/073422, filed on Jan. 20, 2023, which is incorporated herein by reference in its entirety.

### TECHNICAL FIELD

The present disclosure generally relates to the field of intestinal disorders, and in particular, to biomarkers and related methods for detecting inflammatory bowel disease (IBD) and discriminating between Crohn's disease and ulcerative colitis.

### BACKGROUND

Inflammatory bowel disease (IBD) is a term that describes disorders involving long-standing (chronic) inflammation of tissues in the digestive tract, characterized by symptoms including diarrhea, rectal bleeding, abdominal pain, fatigue and weight loss. Additionally, suffering from IBD may also lead to a significant increase of the risk of colon cancer. IBD affect more than 3.5 million people, and their incidence is increasing worldwide, especially in countries undergoing industrialization and westernization. Medical treatment and surgery have all been utilized for IBD treatment, but the recurrence of inflammation after relapse is common, and requires repetitive colonoscopy examination.

There are two main subtypes of IBD: Crohn's disease (CD) and Ulcerative colitis (UC). Identification of an IBD patient as either UC or CD is necessary for treatment and management of the disease. Conventional diagnosis of IBD (for both Crohn's disease and ulcerative colitis) requires the combination of colonoscopy examination and histological examination of the biopsies. The invasive approaches often cause discomfort, pain, or even tissue damage to the patient. As a result, the non-invasive approaches are sometimes preferred. Therefore, it is desirable to develop non-invasive methods for the detection of IBD and discrimination between UC and CD.

### SUMMARY

According to an aspect of the present disclosure, a system for detecting inflammatory bowel disease (IBD) in a subject is provided. The system may include at least one storage device including a set of instructions; and at least one processor in communication with the at least one storage device, wherein when executing the set of instructions, the at least one processor is directed to perform operations including: (a) obtaining, from a quantitative measurement device, quantified abundance of one or more target metabolites in a panel of metabolites in a sample from the subject, wherein the panel of metabolites include metabolites of Table 1; (b) determining a sample score by processing the quantified abundance of each of the one or more target metabolites using a prediction model; and (c) determining whether the subject has IBD by comparing the sample score to a cut-off score.

According to another aspect of the present disclosure, a system for detecting Crohn's disease (CD) in a subject is provided. The system may include at least one storage device including a set of instructions; and at least one processor in communication with the at least one storage device, wherein when executing the set of instructions, the at least one processor is directed to perform operations including: (a) obtaining, from a quantitative measurement device, quantified abundance of one or more target metabolites in a panel of metabolites in a sample from the subject, wherein the panel of metabolites include metabolites of Table 6; (b) determining a sample score by processing the quantified abundance of each of the one or more target metabolites using a prediction model; and (c) determining whether the subject has CD by comparing the sample score to a cut-off score.

According to yet another aspect of the present disclosure, a system for detecting Ulcerative colitis (UC) in a subject is provided. The system may include at least one storage device including a set of instructions; and at least one processor in communication with the at least one storage device, wherein when executing the set of instructions, the at least one processor is directed to perform operations including: (a) obtaining, from a quantitative measurement device, quantified abundance of one or more target metabolites in a panel of metabolites in a sample from the subject, wherein the panel of metabolites include metabolites of Table 11; (b) determining a sample score by processing the quantified abundance of each of the one or more target metabolites using a prediction model; and (c) determining whether the subject has UC by comparing the sample score to a cut-off score.

According to still another aspect of the present disclosure, a system for determining whether a subject has Crohn's disease (CD) or Ulcerative colitis (UC) is provided. The system may include at least one storage device including a set of instructions; and

at least one processor in communication with the at least one storage device, wherein when executing the set of instructions, the at least one processor is directed to perform operations including: (a) obtaining, from a quantitative measurement device, quantified abundance of one or more target metabolites in a panel of metabolites in a sample from the subject, wherein the panel of metabolites include metabolites of Table 16; (b) determining a sample score by processing the quantified abundance of each of the one or more target metabolites using a prediction model; and (c) determining whether the subject has CD or the UC by comparing the sample score to a cut-off score.

According to yet another aspect of the present disclosure, a system for determining whether a subject has Crohn's disease (CD) or Ulcerative colitis (UC) is provided. The system may include: at least one storage device including a set of instructions; and at least one processor in communication with the at least one storage device, wherein when executing the set of instructions, the at least one processor is directed to perform operations including: (a) obtaining, from a quantitative measurement device, quantified abundance of one or more target metabolites in a panel of metabolites in a sample from the subject, wherein the panel of metabolites include metabolites of Table 16; (b) determining a sample score by processing the quantified abundance of each of the one or more target metabolites using a prediction model; and (c) determining whether the subject has CD or the UC by comparing the sample score to a cut-off score.

According to still another aspect of the present disclosure, a system for determining whether a subject has inflammatory bowel disease (IBD) or colorectal polyp is provided. The system may include at least one storage device including a set of instructions; and at least one processor in communication with the at least one storage device, wherein when executing the set of instructions, the at least one processor is directed to perform operations including: (a) obtaining, from a quantitative measurement device, quantified abundance of one or more target metabolites in a panel of metabolites in a sample from the subject, wherein the panel of metabolites include metabolites of Table 21; (b) determining a sample score by processing the quantified abundance of each of the one or more target metabolites using a prediction model; and (c) determining whether the subject has IBD or the colorectal polyp by comparing the sample score to a cut-off score.

According to still another aspect of the present disclosure, a method of detecting inflammatory bowel disease (IBD) in a subject and treating the subject is provided. The method may include (a) obtaining, from a quantitative measurement device, quantified abundance of one or more target metabolites in a panel of metabolites in a sample from the subject, wherein the panel of metabolites include the metabolites of Table 1; (b) determining a sample score by processing the quantified abundance of each of the one or more target metabolites using a prediction model; (c) determining whether the subject has IBD by at least comparing the sample score to a cut-off score; and (d) in response to determining that the subject has IBD, applying a treatment to the subject, wherein the treatment includes at least one of conducting a surgery for the subject or administering anti-IBD therapeutics to the subject. In some embodiments, a use of one or more target metabolites in generating a trained machine-learning model for determining whether a subject has IBD is provided. The one or more target metabolites include at least one, two, three, or all of metabolites in Table 1.

According to another aspect of the present disclosure, a method of detecting Ulcerative colitis (UC) in a subject and treating the subject is provided. The method may include: (a) obtaining, from a quantitative measurement device, quantified abundance of one or more target metabolites in a panel of metabolites in a sample from the subject, wherein the panel of metabolites include the metabolites of Table 6; (b) determining a sample score by processing the quantified abundance of each of the one or more target metabolites using a prediction model; (c) determining whether the subject has UC by at least comparing the sample score to a cut-off score; and (d) in response to determining that the subject has UC, applying a treatment to the subject, wherein the treatment includes at least one of conducting a surgery for the subject or administering anti-UC therapeutics to the subject.

According to still another aspect of the present disclosure, a method of detecting Ulcerative colitis (UC) in a subject and treating the subject is provided. The method may include: (a) obtaining, from a quantitative measurement device, quantified abundance of one or more target metabolites in a panel of metabolites in a sample from the subject, wherein the panel of metabolites include the metabolites of Table 11; (b) determining a sample score by processing the quantified abundance of each of the one or more target metabolites using a prediction model; (c) determining whether the subject has UC by at least comparing the sample score to a cut-off score; and (d) in response to determining that the subject has UC, applying a treatment to the subject, wherein the treatment includes at least one of conducting a surgery for the subject or administering anti-UC therapeutics to the subject.

According to yet another aspect of the present disclosure, a method of determining whether a subject has Crohn's disease (CD) or Ulcerative colitis (UC) in a subject and treating the subject is provided. The method includes: comprising: (a) obtaining, from a quantitative measurement device, quantified abundance of one or more target metabolites in a panel of metabolites in a sample from the subject, wherein the panel of metabolites include the metabolites of Table 16; (b) determining a sample score by processing the quantified abundance of each of the one or more target metabolites using a prediction model; (c) determining whether the subject has IBD by at least comparing the sample score to a cut-off score; and (d) in response to determining that the subject has IBD, applying a treatment to the subject, wherein the treatment includes at least one of conducting a surgery for the subject or administering anti-IBD therapeutics to the subject.

According to still another aspect of the present disclosure, a method of determining whether a subject has Crohn's disease (CD) or Ulcerative colitis (UC) in a subject and treating the subject is provided. The method may include: (a) obtaining, from a quantitative measurement device, quantified abundance of one or more target metabolites in a panel of metabolites in a sample from the subject, wherein the panel of metabolites include the metabolites of Table 16; (b) determining a sample score by processing the quantified abundance of each of the one or more target metabolites using a prediction model; (c) determining whether the subject has IBD by at least comparing the sample score to a cut-off score; and (d) in response to determining that the subject has IBD, applying a treatment to the subject, wherein the treatment includes at least one of conducting a surgery for the subject or administering anti-IBD therapeutics to the subject.

In some embodiments, a use of one or more target metabolites in generating a trained machine-learning model for determining whether a subject has CD. The one or more target metabolites include at least one, two, three, four, or five of metabolites in Table 6.

In some embodiments, a use of one or more target metabolites in generating a trained machine-learning model for determining whether a subject has UC is provided. The one or more target metabolites include at least one, two, three, or ten of metabolites in Table 11.

In some embodiments, a use of one or more target metabolites in generating a trained machine-learning model for determining whether a subject has CD or UC is provided.

In some embodiments, a use of one or more target metabolites in generating a trained machine-learning model for determining whether a subject has IBD or colorectal polyp. The one or more target metabolites include at least one, two, three, four, or all of metabolites in Table 21.

In some embodiments, a use of one or more target metabolites for preparing a kit for detecting IBD in a subject, the one or more target metabolites including at least one, two, three, or all of metabolites in Table 1.

In some embodiments, a use of one or more target metabolites for preparing a kit for detecting CD in a subject is provided. The one or more target metabolites may include at least one, two, three, or all of metabolites in Table 6.

In some embodiments, a use of one or more target metabolites for preparing a kit for detecting UC in a subject, the one or more target metabolites including at least o one, two, three, or all of metabolites in Table 11.

In some embodiments, a use of one or more target metabolites for preparing a kit for determining whether a subject has CD or UC is provided. The one or more target metabolites may include one, two, or all of metabolites in Table 16.

In some embodiments, a use of one or more target metabolites for preparing a kit for determining whether a subject has IBD or colorectal polyp is provided. The one or more target metabolites including at least one, two, three, or all of metabolites in Table 21.

In some embodiments, a kit for detecting IBD in a subject is provided. The kit may include one or more target metabolites, and the one or more target metabolites include at least one, two, three, or all of metabolites in Table 1.

In some embodiments, a kit for detecting CD in a subject is provided. The kit includes one or more target metabolites, and the one or more target metabolites include at least one, two, three, or all of metabolites in Table 6.

In some embodiments, a kit for detecting UC in a subject is provided. The kit may include one or more target metabolites, wherein the one or more target metabolites include at least one, two, three, four, five, or all of metabolites in Table 11.

In some embodiments, a kit for determining whether a subject has CD or UC in a subject is provided. The kit may include one or more target metabolites, wherein the one or more target metabolites include at least one, two, or all of metabolites in Table 16.

In some embodiments, a kit for determining whether a subject has IBD or colorectal polyp is provided. The kit may include one or more target metabolites, wherein the one or more target metabolites include at least one, two, three, or all of metabolites in Table 21.

Additional features will be set forth in part in the description which follows, and in part will become apparent to those skilled in the art upon examination of the following and the accompanying drawings or may be learned by production or operation of the examples. The features of the present disclosure may be realized and attained by practice or use of various aspects of the methodologies, instrumentalities, and combinations set forth in the detailed examples discussed below.

### BRIEF DESCRIPTION OF THE DRAWINGS

The present disclosure is further described in terms of exemplary embodiments. These exemplary embodiments are described in detail with reference to the drawings. It should be noted that the drawings are not to scale. These embodiments are non-limiting exemplary embodiments, in which like reference numerals represent similar structures throughout the several views of the drawings, and wherein:

FIG. 1 is a schematic diagram illustrating an exemplary system for detecting IBD in a subject according to some embodiments of the present disclosure;

FIG. 2A is a Principal Component Analysis (PCA) plot showing discriminations of serum metabolomic states of samples from Normal (N, blue), CD (red) and UC patients (green) based on metabolites that showed significant alternation either between normal and IBD, or between UC and CD patients;

FIG. 2B is a Venn diagram showing overlaps among the three significantly altered and annotated serum metabolites lists (N vs. UC; N vs. CD; CD vs. UC);

FIG. 3 is a Venn diagram showing overlaps among the three significantly altered gut microbiome lists (N vs. UC; N vs. CD; CD vs. UC);

FIG. 4 is a PCA plot showing discriminations of serum metabolomic states of samples from Normal (N, blue), CD (red) and UC patients (green) based on metabolites that both gut-microbiome associated and also showed significant alternation either between normal and IBD, or between UC and CD patients;

FIG. 5A shows the performance of a prediction model for discriminating between negative subjects (including normal subjects) and positive subjects (including subjects having UC) in the discovery cohort;

FIG. 5B shows the performance of a prediction model for discriminating between negative subjects (including normal subjects) and positive subjects (including subjects having CD) in the discovery cohort;

FIG. 5C shows the performance of a prediction model for discriminating between subjects having UC and subjects having CD;

FIG. 5D is a PCA plot of the prediction model for discriminating between normal subjects and subjects having UC;

FIG. 5E is a PCA plot of the prediction model for discriminating between normal subjects and subjects having CD;

FIG. 5F is a PCA plot of the prediction model for discriminating between subjects having UC and subjects having CD;

FIG. 6A shows the performance of a prediction model for discriminating between subjects having UC and normal subjects in the training set;

FIG. 6B shows the performance of a prediction model for discriminating between subjects having UC and normal subjects in the testing set;

FIG. 6C is a PCA plot of the prediction model for discriminating between subjects having UC and normal subjects in the training set;

FIG. 6D is a PCA plot of the prediction model for discriminating between subjects having UC and normal subjects in the testing set;

FIG. 7A shows the performance of a prediction model for discriminating between subjects having CD and normal subjects in the training set;

FIG. 7B shows the performance of a prediction model for discriminating between subjects having CD and normal subjects in the testing set;

FIG. 7C is a PCA plot of the prediction model for discriminating between subjects having CD and normal subjects in the training set;

FIG. 7D is a PCA plot of the prediction model for discriminating between subjects having CD and normal subjects in the testing set;

FIG. 8A shows the performance of a prediction model for discriminating between subjects having CD and subjects having UC in the training set;

FIG. 8B shows the performance of a prediction model for discriminating between subjects having CD and subjects having UC in the testing set;

FIG. 8C is a PCA plot of the prediction model for discriminating between subjects having CD and subjects having UC in the training set;

FIG. 8D is a PCA plot of the prediction model for discriminating between subjects having CD and subjects having UC in the testing set;

FIG. 9A shows the performance of a prediction model for discriminating between non-IBD subjects and subjects having IBD in the training set;

FIG. 9B shows the performance of a prediction model for discriminating between non-IBD subjects and subjects having IBD in the testing set;

FIG. 9C is a PCA plot of the prediction model for discriminating between subjects having CD and subjects having UC in the training set;

FIG. 9D is a PCA plot of the prediction model for discriminating between subjects having CD and subjects having UC in the testing set;

FIG. 10A shows the performance of a prediction model for discriminating between subjects having IBD and subjects having colorectal polyps in the training set;

FIG. 10B shows the performance of a prediction model for discriminating between subjects having IBD and subjects having colorectal polyps in the testing set;

FIG. 10C is a PCA plot of the prediction model for discriminating between subjects having IBD and subjects having colorectal polyps in the training set; and

FIG. 10D is a PCA plot of the prediction model for discriminating between subjects having IBD and subjects having colorectal polyps in the testing set.

### DETAILED DESCRIPTION

The following description is presented to enable any person skilled in the art to make and use the present disclosure and is provided in the context of a particular application and its requirements. Various modifications to the disclosed embodiments will be readily apparent to those skilled in the art, and the general principles defined herein may be applied to other embodiments and applications without departing from the spirit and scope of the present disclosure. Thus, the present disclosure is not limited to the embodiments shown but is to be accorded the widest scope consistent with the claims.

The terminology used herein is to describe particular example embodiments only and is not intended to be limiting. As used herein, the singular forms “a,” “an,” and “the” may be intended to include the plural forms as well, unless the context clearly indicates otherwise. It will be further understood that the terms “comprises,” “comprising,” “includes,” and/or “including” when used in this specification, specify the presence of stated features, integers, steps, operations, elements, and/or components, but do not preclude the presence or addition of one or more other features, integers, steps, operations, elements, components, and/or groups thereof.

These and other features, and characteristics of the present disclosure, as well as the methods of operation and functions of the related elements of structure and the combination of parts and economies of manufacture, may become more apparent upon consideration of the following description with reference to the accompanying drawing(s), all of which form a part of this specification. It is to be expressly understood, however, that the drawing(s) is for the purpose of illustration and description only and are not intended to limit the scope of the present disclosure. It is understood that the drawings are not to scale.

As used herein, the term “subject” of the present disclosure refers to any human or non-human animal. Exemplary non-human animals may include Mammalia (such as chimpanzees and other apes and monkey species), farm animals (such as cattle, sheep, pigs, goats, and horses), domestic mammals (such as dogs and cats), laboratory animals (such as mice, rats, and guinea pigs), or the like. In some embodiments, the subject is a human. The term “normal subject” refers to a subject who is not suffering from IBD or colorectal polyps.

The present disclosure provides panels of metabolites for use as diagnostic biomarkers for detecting IBD in a subject and panels of metabolites for use as diagnostic biomarkers for discriminating between UC and CD, which are the main subtypes of IBD. Panels of metabolites for used as diagnostic biomarkers for discriminating between IBD and colorectal polyp are also provided. These diagnostic biomarkers are serum metabolites associated with gut microbiome. Systems and methods for detecting IBD and for discriminating between UC and CD using the panels of metabolites are also provided. As compared with conventional methods for detecting IBD, (e.g., a method using a colonoscope and/or a biopsy test), the methods provided by the present disclosure are non-invasive and are capable of effectively distinguishing subjects having IBD from non-IBD subjects, and discriminating between patients having UC and patients have CD.

In some embodiments, the diagnostic biomarkers provided by the present disclosure may be used to monitor status of IBD patients, for example, disease alleviation, or recurrence via non-invasive blood test, instead of relying on invasive colonoscopy. In some embodiments, the diagnostic biomarkers provided by the present disclosure may be used for conducting precision treatment on IBD patients. Specifically, the diagnositic biomarkers may be used to distinguish UC and CD subtypes and may help determine the corresponding treatment. Real-time monitoring disease status during the treatment may be conducted, which helps determine whether the patient is responsive to current treatment.

According to an aspect of the present disclosure, systems for detecting IBD and systems for discriminating between UC and CD are provided. Specifically, the systems may include a system for detecting IBD, a system for detecting UC, a system for detecting CD, a system for discriminating between UC and CD, and a system for discriminating between IBD and colorectal polyp. A major difference between these systems provided by the present disclosure is that these systems utilize different panels of metabolites.

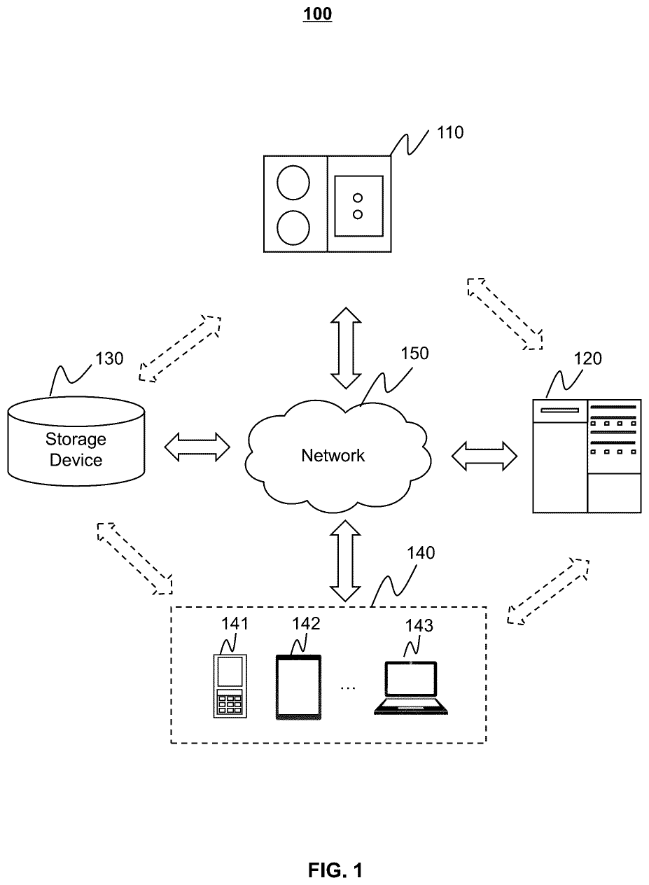

FIG. 1 is a schematic diagram illustrating an exemplary system for detecting IBD in a subject according to some embodiments of the present disclosure. In some embodiments, the method for detecting intestinal disorders in a subject may be implemented on the system 100. As illustrated, the system 100 may include a quantitative measurement device 110, a processing device 120, a storage device 130, a terminal device 140, and a network 150. The components of the system 100 may be connected in various ways. Merely by way of example, as illustrated in FIG. 1, the quantitative measurement device 110 may be connected to the processing device 120 directly as indicated by the bi-directional arrow in dotted lines linking the quantitative measurement device 110 and the processing device 120, or through the network 150. As another example, the storage device 130 may be connected to the quantitative measurement device 110 directly as indicated by the bi-directional arrow in dotted lines linking the quantitative measurement device 110 and the storage device 130, or through the network 150. As still another example, the terminal device 140 may be connected to the processing device 120 directly as indicated by the bi-directional arrow in dotted lines linking the terminal device 140 and the processing device 120, or through the network 150.

The quantitative measurement device 110 may be configured to measure an abundance of one or more target metabolites for use as diagnostic biomarkers for detecting diagnostic disorders. In some embodiments, the quantitative measurement device 110 may measure the abundance of the one or more target metabolites using a relative quantification approach or an absolute quantification approach. Merely by way of example, the quantitative measurement device 110 may include a mass spectrometer (MS; e.g., liquid chromatography-mass spectrometer, gas chromatography-mass spectrometer; matrix-assisted laser desorption/ionization time-of-flight mass spectrometer), an ultraviolet spectrometer, a High-Performance Liquid Chromatography (HPLC) apparatus, or the like.

The processing device 120 may process data and/or information obtained from the quantitative measurement device 110, the storage device 130, and/or the terminal device 140. In some embodiments, the processing device 120 may be used to process the quantified abundance of the one or more target metabolites for evaluating whether the subject has. For example, the processing device 120 may obtain a prediction model. The quantified abundance of the one or more target metabolites may be inputted into the prediction model to obtain a sample score for the subject. The processing device 120 may further evaluate whether the subject has IBD by comparing the sample score to a cut-off value of the prediction model. In some embodiments, the processing device 120 may determine the quantified abundance of the one or more target metabolites based on data acquired by the quantitative measurement device 110.

In some embodiments, the processing device 120 may be a single server or a server group. The server group may be centralized or distributed. In some embodiments, the processing device 120 may be local or remote. For example, the processing device 120 may access information and/or data from the quantitative measurement device 110, the storage device 130, and/or the terminal device 140 via the network 150. As another example, the processing device 120 may be directly connected to the quantitative measurement device 110, the terminal device 140, and/or the storage device 130 to access information and/or data. In some embodiments, the processing device 120 may be implemented on a cloud platform. For example, the cloud platform may include a private cloud, a public cloud, a hybrid cloud, a community cloud, a distributed cloud, an inter-cloud, a multi-cloud, or the like, or a combination thereof. In some embodiments, the processing device 120 may be part of the terminal device 140. In some embodiments, the processing device 120 may be part of the quantitative measurement device 110.

The storage device 130 may store data, instructions, and/or any other information. In some embodiments, the storage device 130 may store data obtained from the quantitative measurement device 110, the processing device 120, and/or the terminal device 140. The data may include quantified abundance of the one or more target metabolites of the subject and/or the prediction model for processing the quantified abundance, etc. In some embodiments, the storage device 130 may store data and/or instructions that the processing device 120 may execute or use to perform exemplary methods described in the present disclosure. In some embodiments, the storage device 130 may include a mass storage, removable storage, a volatile read-and-write memory, a read-only memory (ROM), or the like, or any combination thereof. Exemplary mass storage may include a magnetic disk, an optical disk, a solid-state drive, etc. Exemplary removable storage may include a flash drive, a floppy disk, an optical disk, a memory card, a zip disk, a magnetic tape, etc. Exemplary volatile read-and-write memories may include a random-access memory (RAM). Exemplary RAM may include a dynamic RAM (DRAM), a double date rate synchronous dynamic RAM (DDR SDRAM), a static RAM (SRAM), a thyristor RAM (T-RAM), and a zero-capacitor RAM (Z-RAM), etc. Exemplary ROM may include a mask ROM (MROM), a programmable ROM (PROM), an erasable programmable ROM (EPROM), an electrically erasable programmable ROM (EEPROM), a compact disk ROM (CD-ROM), and a digital versatile disk ROM, etc. In some embodiments, the storage device 130 may be implemented on a cloud platform. Merely by way of example, the cloud platform may include a private cloud, a public cloud, a hybrid cloud, a community cloud, a distributed cloud, an inter-cloud, a multi-cloud, or the like, or any combination thereof. some embodiments, the storage device 130 may be connected to the network 150 to communicate with one or more other components (e.g., the processing device 120, the terminal device 140) of the system 100. One or more components of the system 100 may access the data or instructions stored in the storage device 130 via the network 150. In some embodiments, the storage device 130 may be integrated into the quantitative measurement device 110 or the processing device 120.

The terminal device 140 may be connected to and/or communicate with the quantitative measurement device 110, the processing device 120, and/or the storage device 130. In some embodiments, the terminal device 140 may include a mobile device 141, a tablet computer 142, a laptop computer 143, or the like, or any combination thereof. For example, the mobile device 141 may include a mobile phone, a personal digital assistant (PDA), or the like, or any combination thereof. In some embodiments, the terminal device 140 may include an input device, an output device, etc. The input device may include alphanumeric and other keys that may be input via a keyboard, a touchscreen (e.g., with haptics or tactile feedback), a speech input, an eye-tracking input, a brain monitoring system, or any other comparable input mechanism. Other types of the input device may include a cursor control device, such as a mouse, a trackball, or cursor direction keys, etc. The output device may include a display, a printer, or the like, or any combination thereof. The terminal device 140 may be used to present information to a user and/or convey a user instruction to other components of the system 100. For example, the user (e.g., a doctor) may instruct the quantitative measurement device 110 to start quantifying the abundance of the one or more target metabolites via the terminal device 140. As another example, the user may view an evaluation result regarding whether the subject has IBD via the terminal device 140.

The network 150 may include any suitable network that can facilitate the exchange of information and/or data for the system 100. In some embodiments, one or more components (e.g., the quantitative measurement device 110, the processing device 120, the storage device 130, the terminal device 140) of the system 100 may communicate information and/or data with one or more other components of the system 100 via the network 150.

It should be noted that the system 100 is only provided for illustration purposes. The system for detecting UC, the system for detecting CD, the system for discriminating between UC and CD, and the system for discriminating between IBD and colorectal polyp may have components that are similar to the system 100.

According to an aspect of the present disclosure, a panel of metabolites for detecting whether a subject has IBD is provided. In some embodiments, the group of metabolites may include one or more target metabolites correlated with IBD. The one or more target metabolites may be serum metabolites that exhibit significant differentiation between a positive group of subjects (IBD patients) and a negative group of subjects (normal people). More details regarding the determination of the one or more target metabolites may be found elsewhere in the present disclosure, e.g., Example 1.

In some embodiments, the abundance of the metabolite(s) in a sample obtained from a normal subject may be different from the abundance of the metabolite(s) in a sample obtained from a subject that has IBD. As used herein, the term “abundance” refers to the quantity or amount of a substance in a certain sample. The sample may be a fluid sample such as a serum sample. Merely by way of example, the one or more target metabolites may be present in the serum and may be referred to as “serum metabolites”.

In some embodiments, to measure the abundance of a metabolite, the concentration or amount of the metabolite in the fluid sample may be measured. The abundance of each of the one or more target metabolites may be quantified by a quantitative measurement device using a relative quantification approach or an absolute quantification approach. For example, the abundance of a metabolite may be a relative abundance determined based on a normalized value or a relative value with respect to a control. In some embodiments, the control may be the precise concentration or amount of a set of chemicals that are artificially added into a subject, such as spike-in control. Alternatively, the control may be the concentration or amount of the same metabolite of a sample obtained from a pool of subjects who do not have IBD and is considered physically healthy. Alternatively, the abundance of the metabolite may be an absolute abundance that directly reflects the level of the metabolite in the subject. In some embodiments, the abundance of the metabolite may be obtained by mass spectrometry, chromatography (e.g., HPLC), and any other appropriate techniques.

Table 1 shows an exemplary group of metabolites that can be used for detecting IBD. Each of the metabolites, which are biomarkers, has shown a strong and reliable correlation with the presence of IBD. In some embodiments, the group of metabolites provided by the present disclosure may include one or more target metabolites of Table 1. In some embodiments, the group of metabolites may include at least one of the metabolites of Table 1. In some embodiments, the group of metabolites may include at least two of the metabolites of Table 1. In some embodiments, the group of metabolites may include at least 3, 4, or 5 of the metabolites of Table 1. As another example, the group of metabolites may include all of the metabolites of Table 1.

TABLE 1

| | | | | | Delta |
|---|---|---|---|---|---|
| No. | Meta ID | MASS (+/−) | | Compound | (ppm) |
| 1 | BN029 | 187.098 | (−) | Azelaic acid | 0 |
| 2 | BP013 | 303.232 | (+) | 17-Alpha- | 0 |
| | | | | Methyltestosterone | |
| 3 | C004 | 267.073 | (−) | C10H12N4O5 | 2 |
| 4 | C008 | 327.256 | (−) | C19H36O4 | 5 |
| 5 | C019 | 447.312 | (−) | C27H44O5 | 1 |
| 6 | C027 | 481.354 | (−) | C31H46O4 | 45 |
| 7 | C147 | 506.323 | (+) | C25H47NO9 | 18 |
| 8 | C148 | 508.340 | (+) | C25H50NO7P | 1 |
| 9 | X285 | 512.336 | (−) | C26H43NO7S | 131 |
| 10 | X403 | 239.092 | (−) | C13H12N4O | 8 |
| 11 | X508 | 212.020 | (+) | C7H2F5NO | 33 |

For example, mass spectrometry (or other techniques) may be used to quantify the abundance of one or more target metabolites in a panel of metabolites in a sample. The abundance of each metabolite that has been quantified can be processed and used to detect IBD and/or facilitate the treatment of IBD in the subject. In some embodiments, any one of the metabolites in Table 1 can be quantified and used for these purposes. In some embodiments, any two, three, or four metabolites in Table 1 can be quantified and used for these purposes. In some embodiments, any five, ten, or fifteen metabolites in Table 1 can be quantified and used for these purposes. In some embodiments, all the metabolites in Table 1 can be quantified and used for these purposes.

In some embodiments, the one or more target metabolites for detecting IBD and/or facilitating the treatment of IBD may include at least one metabolite of Table 1 and at least one metabolite of Table 2. Each of the metabolites in Table 2 is found to be closely correlated with the presence of IBD. In some embodiments, the one or more target metabolites may further include 1, 2, 3, or all of the metabolites of the metabolites in Table 2. For example, the one or more target metabolites may include one metabolite in Table 1 and one metabolite in Table 2. As another example, the one or more target metabolites may include one metabolite in Table A and two metabolites in Table 2. As yet another example, the one or more target metabolites may include two metabolites in Table 1 and one metabolite in Table 2. See, e.g., Example 2. Similarly, any combinations of one or more metabolites in Table 1 and one or more metabolites in Table 2 may be used to achieve the same purposes. In some embodiments, one or more of the metabolites in Table 2 may be used, independently from the metabolites listed in Table 1, for detecting and/or facilitating the treatment of IBD in the subject.

TABLE 2

| | | | | | Delta |
|---|---|---|---|---|---|
| No. | Meta ID | MASS (+/−) | | Compound | (ppm) |
| 1 | BP002 | 177.102 | (+) | S-(−)-Cotinine | 0 |
| 2 | BP011 | 316.248 | (+) | Decanoyl-L- | 0 |
| | | | | carnitine | |
| 3 | X407 | 314.103 | (−) | C17H17NO5 | 1 |
| 4 | X293 | 204.067 | (−) | C11H11NO3 | 2 |
| 5 | BN021 | 405.265 | (−) | 3- | 0 |
| | | | | Dehydrocholic | |
| | | | | Acid | |
| 6 | C119 | 337.273 | (+) | C21H36O3 | 2 |
| 7 | BN017 | \ | | Epitestosterone | 0 |
| | | | | Sulfate | |
| 8 | DS04 | 464.302 | (−) | GCA | 0 |
| | | | | (Glycocholic | |
| | | | | Acid Hydrate) | |
| 9 | X024 | 369.174 | (−) | C19H30O5S | 0 |

In some embodiments, the one or more target metabolites for detecting IBD may include a metabolite of Table 3. In some embodiments, the metabolite in Table 3 may be used, in addition to the one or more metabolites listed in Table 1 and/or one or more metabolites listed in Table 2, for detecting IBD and/or facilitating the treatment of IBD in the subject.

TABLE 3

| | | | | Delta |
|---|---|---|---|---|
| No. | Meta ID | MASS (+/−) | Compound | (ppm) |
| 1 | X082 | 353.212 (−) | C19H26N6O | 7 |

In some embodiments, the one or more target metabolites provided by the present disclosure may include at least one metabolite of Table 4. Each of the metabolites in Table 4 is found to be correlated with the presence of IBD. In some embodiments, one or more of the metabolites in Table 4 may be used, in addition to the one or more metabolites listed in Table 1 and/or one or more metabolites listed in Table 2, for detecting IBD and/or facilitating the treatment of IBD in the subject. As another example, one or more of the metabolites in Table 23 may be used, in addition to the one or more metabolites listed in Table 1, one or more metabolites listed in Table 2, and one or more metabolites listed in Table 3 for the same purposes. In some embodiments, the abundance of 1, 2, 3 or all the metabolites of the metabolites in Table 4 may be quantified.

TABLE 4

| | Meta | | | | Delta |
|---|---|---|---|---|---|
| No | ID | MASS (+/−) | | Compound | (ppm) |
| 1 | BN001 | 319.228 | (−) | 5_HETE | 0 |
| 2 | BN003 | 319.228 | (−) | 8_HETE | 0 |
| 3 | BN004 | 319.228 | (−) | 9_HETE | 0 |
| 4 | BN006 | 319.228 | (−) | 12_HETE | 0 |
| 5 | BN012 | 343.228 | (−) | 14(S)-HDHA | 0 |
| 6 | BN013 | 343.228 | (−) | 17(S)_HDHA | 0 |
| 7 | BN015 | 350.210 | (−) | Sphingosine-1-phosphate (d16:1) | 0 |
| 8 | BN016 | 313.239 | (−) | Octadecane dioic acid | 0 |
| 9 | BN020 | 389.270 | (−) | 3α-Hydroxy-6-OXO-5α-Cholan-24-OIC | 0 |
| | | | | Acid | |
| 10 | BN022 | 405.265 | (−) | 5α-Cholanic Acid-3α, 7β-Diol-6-One | 0 |
| 11 | BN023 | 301.218 | (−) | Eicosapentaenoic Acid | 0 |
| 12 | BN027 | 480.310 | (−) | 1-Stearoyl-2-Hydroxy-sn-Glycero-3- | 0 |
| | | | | Phosphoethanolamine | |
| 13 | BN028 | 159.067 | (−) | Pimelic acid | 0 |
| 14 | BN030 | 303.233 | (−) | Arachidonic acid | 0 |
| 15 | BP003 | 379.284 | (+) | 2-Arachidonoyl Glycerol | 0 |
| 16 | BP007 | 286.201 | (+) | trans-2-octenoyl-I-carnitine | 0 |
| 17 | BP009 | 355.284 | (+) | 1-Linoleoyl-rac-glycerol | 0 |
| 18 | C016 | 427.163 | (−) | C24H23F3N2O2 | 2 |
| 19 | C017 | 439.379 | (−) | C27H52O4 | 0 |
| 20 | C021 | 468.308 | (−) | C29H43NO2S | 30 |
| 21 | C026 | 480.310 | (−) | C25H43N3O6 | 4 |
| 22 | C031 | 540.331 | (−) | C34H43N3O3 | 14 |
| 23 | C033 | 581.241 | (−) | C33H34N4O6 | 1 |
| 24 | C035 | 590.346 | (−) | C33H45N5O5 | 19 |
| 25 | C041 | 499.288 | (−) | C26H44O9 | 7 |
| 26 | C043 | 511.302 | (−) | C31H44O6 | 9 |
| 27 | C102 | 181.072 | (+) | C7H8N4O2 | 1 |
| 28 | C110 | 286.201 | (+) | C15H27NO4 | 1 |
| 29 | C112 | 315.134 | (+) | C15H17F3N2O2 | 9 |
| 30 | C116 | 330.263 | (+) | C18H35NO4 | 2 |
| 31 | C120 | 355.283 | (+) | C21H38O4 | 4 |
| 32 | C131 | 468.308 | (+) | C22H46NO7P | 1 |
| 33 | C132 | 480.134 | (+) | C23H21N5O5S | 1 |
| 34 | C135 | 195.087 | (+) | C8H10N4O2 | 2 |
| 35 | C136 | 287.204 | (+) | C19H26O2 | 14 |
| 36 | C137 | 302.215 | (+) | C19H27NO2 | 11 |
| 37 | C139 | 341.306 | (+) | C21H40O3 | 2 |
| 38 | C144 | 357.280 | (+) | C24H36O2 | 3 |
| 39 | C145 | 464.314 | (+) | C23H46NO6P | 2 |
| 40 | C146 | 482.324 | (+) | C23H48NO7P | 0 |
| 41 | C149 | 508.340 | (+) | C28H45NO7 | 26 |
| 42 | C150 | 530.324 | (+) | C27H48NO7P | 1 |
| 43 | DS01 | 407.281 | (−) | CA (Cholic Acid) | 0 |
| 44 | DS02 | 391.286 | (−) | CDCA (Chenodeoxycholic Acid) | 0 |
| 45 | DS05 | 448.307 | (−) | GCDCA (Glycochenodeoxycholic Acid) | 0 |
| 46 | DS10 | 391.286 | (−) | UDCA (Ursodeoxycholic Acid) | 0 |
| 47 | DS11 | \ | | 5β-CAA-3β, 12α-2K | 0 |
| 48 | X004 | 239.092 | (−) | C12H16O5 | 2 |
| 49 | X006 | 263.104 | (−) | C13H16N2O4 | 1 |
| 50 | X013 | 313.238 | (−) | C18H34O4 | 1 |
| 51 | X016 | 319.228 | (−) | C20H32O3 | 0 |
| 52 | X023 | 367.158 | (−) | C19H28O5S | 1 |
| 53 | X055 | 526.315 | (−) | C27H46NO7P | 40 |
| 54 | X066 | 592.362 | (−) | C29H56NO9P | 0 |
| 55 | X154 | 302.196 | (+) | C16H23N5O | 5 |
| 56 | X160 | 316.247 | (+) | C17H33NO4 | 4 |
| 57 | X166 | 352.224 | (+) | C16H34NO5P | 2 |
| 58 | X183 | 490.300 | (+) | C27H41F2N5O | 72 |
| 59 | X188 | 542.324 | (+) | C28H48NO7P | 0 |
| 60 | X278 | 289.106 | (−) | C11H18N2O7 | 6 |
| 61 | X280 | 447.312 | (−) | C30H40O3 | 48 |
| 62 | X281 | 447.312 | (−) | C27H44O5 | 1 |
| 63 | X286 | 512.336 | (−) | C29H41F2N5O | 30 |
| 64 | X289 | 536.299 | (−) | C29H47NO8 | 45 |
| 65 | X292 | 187.007 | (−) | C7H804S | 0 |
| 66 | X401 | 222.114 | (−) | C12H17NO3 | 2 |
| 67 | X408 | 317.212 | (−) | C20H30O3 | 1 |
| 68 | X409 | 335.259 | (−) | C20H32O4 | 108 |
| 69 | X411 | 345.243 | (−) | C22H34O3 | 2 |
| 70 | X513 | 305.247 | (+) | C20H32O2 | 2 |
| 71 | X519 | 364.084 | (+) | C11H18N5O7P | 49 |
| 72 | X525 | 563.427 | (+) | C34H58O6 | 6 |
| 73 | X664 | 413.201 | (−) | C23H30N2O5 | 18 |
| 74 | X657 | 212.003 | (−) | C8H7NO4S | 1 |
| 75 | X682 | 368.087 | (+) | C16H15F2N3O3S | 3 |
| 76 | X667 | 453.321 | (−) | C26H46O6 | 2 |
| 77 | X677 | 251.127 | (+) | C14H18O4 | 2 |
| 78 | X653 | 194.046 | (−) | C9H9NO4 | 1 |
| 79 | X684 | 399.237 | (+) | C21H34O7 | 2 |
| 80 | X678 | 285.206 | (+) | C16H28O4 | 2 |
| 81 | X681 | 337.273 | (+) | C21H36O3 | 2 |
| 82 | X686 | 510.355 | (+) | C25H52NO7P | 1 |

In some embodiments, the one or more target metabolites for detecting IBD may include one or more metabolite combinations shown in Table 5.

TABLE 5

| Meta ID | AUC | Sens | Spec |
|---|---|---|---|
| BN017, DS04 | 0.76 | 0.55 | 0.91 |
| BN021, X024, X082 | 0.78 | 0.57 | 0.93 |
| BN017, C119, DS04 | 0.76 | 0.59 | 0.9 |

For the metabolite annotations of Meta IDs used in the present disclosure, please refer to Table 31.

In some embodiments, the one or more target metabolites for detecting IBD may include the one or more metabolic combinations shown in Table 5 but exclude any metabolites shown in Table 1. Alternatively, the one or more target metabolites may include the one or more metabolic combinations shown in Table 5 and at least one metabolite selected from the metabolites in Table s 1-4.

A method of detecting IBD in a subject is provided. In some embodiments, the method may include: (a) obtaining, from a quantitative measurement device, quantified abundance of one or more target metabolites in a panel of metabolites in a sample from the subject; (b) determining a sample score by processing the quantified abundance of each of the one or more target metabolites using a prediction model; and (c) estimating whether the subject has IBD by comparing the sample score to a cut-off score. The description of the one or more target metabolites may be found earlier in the present disclosure. For example, the one or more target metabolites may include at least one metabolite in the metabolites of Table 1. As another example, the one or more target metabolites may include at least one metabolite in the metabolites of Table 1, at least one metabolite selected from the metabolites of Table 2-4, and/or one or more metabolic combinations in Table 5.

In some embodiments, the method of detecting IBD may be followed by a treatment for IBD. For example, the treatment may include a surgery for removing a diseased bowel part and/or administering anti-IBD therapeutics to the subject. For example, the anti-IBD treatment may include corticosteroids, anti-inflammatory agents, tumor necrosis factor inhibitors, immunosuppressants, antibiotics, and Alpha 4 Integrin inhibitors.

In some embodiments, the abundance of the one or more components of the panel of metabolites may be measured using mass spectrometry (MS; e.g., liquid chromatography-mass spectrometry (LC-MS), gas chromatography-mass spectrometry (GC-MS); matrix-assisted laser desorption/ionization time-of-flight mass spectrometry (MALDI-TOF MS)), ultraviolet spectrometry, High-Performance Liquid Chromatography (HPLC), or the like. In some embodiments, step b) may further include normalizing the abundance of each of the metabolites quantified in step (a), and determining the sample score by processing the normalized abundance with a prediction model.

In some embodiments, the determination of the sample score may be implemented on a computing device (e.g., the processing device 120 illustrated in FIG. 1). The computing device may obtain a prediction model for determining the sample score. The abundance of each of the metabolites quantified in step a) may be inputted into the prediction model. The prediction model may process the abundance (e.g., a relative abundance or an absolute abundance) of each of the metabolites quantified in step a) and output the sample score. Merely by way of example, the abundance of each of the metabolites quantified in step a) may be quantified by measuring the concentration of each of the metabolites. In some embodiments, the measured concentration may be normalized. For instance, the measured concentration may be divided by a total concentration of all metabolites in the sample. The sample score may indicate a probability that the subject has IBD.

In some embodiments, the prediction model may be a trained machine-learning model. For example, the prediction model may be generated using a gradient boosting decision tree (GBDT) algorithm, a decision tree algorithm, a Random Forest algorithm, a logistic regression algorithm, a support vector machine (SVM) algorithm, a Naive Bayesian algorithm, an AdaBoost algorithm, a K-a nearest neighbor (KNN) algorithm, a Markov Chains algorithm, an XGBoosting algorithm, a deep learning algorithm, a neural network, or the like, or any combination thereof, which is not limited by the present disclosure.

To obtain the prediction model, a preliminary model may be trained using a plurality of training datasets. Each of the plurality of training datasets may include a quantified abundance of a sample metabolite of a reference subject and a label indicating whether the reference subject has IBD or is normal. The plurality of reference subjects may include a plurality of normal subjects who do not have IBD and a plurality of subjects having IBD. Merely by way of example, the label may be a positive label or a negative label. The positive label indicates that the reference subject has IBD, and the negative label indicates that the reference subject is normal. If a reference subject is not suffering from IBD, the corresponding label may be designated as 0 (i.e., as a negative label). If a reference subject has IBD, the corresponding label may be designated as 1 (i.e., as a positive sample). Accordingly, the sample score outputted by the prediction model may be a value between 0 and 1. The closer the sample score is to 1, the higher the probability that the subject has IBD is.

In step c), the sample score is compared to a cut-off score related to the prediction model. As used herein, the term “cut-off value” refers to a dividing point on measuring scales where evaluation results are divided into different categories. In some embodiments, when the sample score is equal to or greater than the cut-off score, the computing device may determine that the subject has IBD. The cut-off value may be determined based on the performance of the prediction model.

In some embodiments, the prediction model may be used to distinguish normal people from IBD patients. In some embodiments, the plurality of reference subjects having IBD may include patents having CD or UC.

In some embodiments, a receiver operating characteristic (ROC) curve may be used to evaluate the performance of the prediction model. The ROC curve may illustrate the diagnostic ability of the prediction model as its cut-off value is varied. The ROC curve is usually generated by plotting the sensitivity against the specificity. An area-under-the-curve (AUC) may be determined based on the ROC curve. The AUC may indicate the probability that a classifier (i.e., the prediction model) will rank a randomly chosen positive instance higher than a randomly chosen negative one.

More descriptions regarding the performance of some exemplary prediction models for detecting IBD may be found in the Examples section.

According to another aspect of the present disclosure, a panel of metabolites for detecting whether a subject has CD is provided. In some embodiments, the group of metabolites may include one or more target metabolites correlated with CD. The one or more target metabolites may be serum metabolites that exhibit significant differentiation between a positive group of subjects (CD patients) and a negative group of subjects (normal people). More details regarding the determination of the one or more target metabolites may be found elsewhere in the present disclosure, e.g., Example 1.

Table 6 shows an exemplary group of metabolites that can be used for detecting CD. Each of the metabolites, which are biomarkers, has shown a strong and reliable correlation with the presence of CD. In some embodiments, the group of metabolites provided by the present disclosure may include one or more target metabolites of Table 6. In some embodiments, the group of metabolites may include at least one of the metabolites of Table 6. In some embodiments, the group of metabolites may include at least two of the metabolites of Table 6. In some embodiments, the group of metabolites may include at least 3, 4, or 5 of the metabolites of Table 6. As another example, the group of metabolites may include all of the metabolites of Table 6.

TABLE 6

| | | | | | Delta |
|---|---|---|---|---|---|
| No. | Meta ID | Compound | MASS (+/−) | | (ppm) |
| 1 | BN029 | Azelaic acid | 187.098 | (−) | 0 |
| 2 | C004 | C10H12N4O5 | 267.073 | (−) | 2 |
| 3 | C019 | C27H44O5 | 447.312 | (−) | 1 |
| 4 | C027 | C31H46O4 | 481.354 | (−) | 45 |
| 5 | X508 | C7H2F5NO | 212.020 | (+) | 33 |

In some embodiments, the one or more target metabolites for detecting CD and/or facilitating the treatment of CD may include at least one metabolite of Table 6 and at least one metabolite of Table 7. Each of the metabolites in Table 7 is found to be closely correlated with the presence of CD. In some embodiments, the one or more target metabolites may further include 1, 2, 3, or all of the metabolites of the metabolites in Table 7. For example, the one or more target metabolites may include one metabolite in Table 6 and one metabolite in Table 7. As another example, the one or more target metabolites may include one metabolite in Table 6 and two metabolites in Table 7. As yet another example, the one or more target metabolites may include two metabolites in Table 6 and one metabolite in Table 7. See, e.g., Example 2. Similarly, any combinations of one or more metabolites in Table 6 and one or more metabolites in Table 7 may be used to achieve the same purposes. In some embodiments, one or more of the metabolites in Table 7 may be used, independently from the metabolites listed in Table 6, for detecting and/or facilitating the treatment of CD in the subject.

TABLE 7
In some embodiments, one or more of the
metabolites in Table 8 may be used,

| | | | | | Delta |
|---|---|---|---|---|---|
| No. | Meta ID | Compound | MASS (+/−) | | (ppm) |
| 1 | X082 | C19H26N6O | 353.212 | (−) | 7 |
| 2 | C146 | C23H48NO7P | 482.324 | (+) | 0 |
| 3 | C036 | C31H57N5O9 | 642.396 | (−) | 19 |
| 4 | C150 | C27H48NO7P | 530.324 | (+) | 1 |
| 5 | X004 | C12H16O5 | 239.092 | (−) | 2 |
| 6 | C009 | C22H20N4O | 355.158 | (−) | 4 |
| 7 | DS02 | CDCA | 391.286 | (−) | 0 |
| | | (Chenodeoxycholic | | | |
| | | Acid) | | | |
| 8 | C147 | C25H47NO9 | 506.323 | (+) | 18 |
| 9 | BP012 | (±)-Hexanoyl carnitine | 261.193 | (+) | 0 |
| | | chloride | | | |

in addition to the one or more metabolites listed in Table 6 and/or one or more metabolites listed in Table 7, for detecting CD and/or facilitating the treatment of CD in the subject. In some embodiments, the abundance of 1, 2, 3 or all the metabolites of the metabolites in Table 8 may be quantified for the same purposes.

TABLE 8

| | | | | | Delta |
|---|---|---|---|---|---|
| No. | Meta ID | Compound | MASS (+/−) | | (ppm) |
| 1 | BN001 | 5_HETE | 319.228 | (−) | 0 |
| 2 | X403 | C13H12N4O | 239.092 | (−) | 8 |

In some embodiments, the one or more target metabolites provided by the present disclosure may include at least one metabolite of Table 9. Each of the metabolites in Table 9 is found to be correlated with the presence of CD. In some embodiments, one or more of the metabolites in Table 9 may be used, in addition to the one or more metabolites listed in Table 6 and/or one or more metabolites listed in Table 7, for detecting CD and/or facilitating the treatment of CD in the subject. As another example, one or more of the metabolites in Table 9 may be used, in addition to the one or more metabolites listed in Table 6, one or more metabolites listed in Table 7, and one or more metabolites listed in Table 8 for the same purposes. In some embodiments, the abundance of 1, 2, 3 or all the metabolites of the metabolites in Table 9 may be quantified.

TABLE 9

| | | | | | Delta |
|---|---|---|---|---|---|
| No. | Meta ID | Compound | MASS (+/−) | | (ppm) |
| 1 | BN003 | 8_HETE | 319.228 | (−) | 0 |
| 2 | BN006 | 12_HETE | 319.228 | (−) | 0 |
| 3 | BN012 | 14(S)-HDHA | 343.228 | (−) | 0 |
| 4 | BN015 | Sphingosine-1- | 350.210 | (−) | 0 |
| | | phosphate (d16:1) | | | |
| 5 | BN021 | 3-Dehydrocholic Acid | 405.265 | (−) | 0 |
| 6 | BN022 | 5α-Cholanic Acid- | 405.265 | (−) | 0 |
| | | 3α,7β-Diol-6-One | | | |
| 7 | BN023 | Eicosapentaenoic Acid | 301.218 | (−) | 0 |
| 8 | BN028 | Pimelic acid | 159.067 | (−) | 0 |
| 9 | BN030 | Arachidonic acid | 303.233 | (−) | 0 |
| 10 | BP003 | 2-Arachidonoyl Glycerol | 379.284 | (+) | 0 |
| 11 | BP009 | 1-Linoleoyl-rac-glycerol | 355.284 | (+) | 0 |
| 12 | BP013 | 17-Alpha- | 303.232 | (+) | 0 |
| | | Methyltestosterone | | | |
| 13 | C006 | C18H32O3 | 295.229 | (−) | 2 |
| 14 | C008 | C19H36O4 | 327.256 | (−) | 5 |
| 15 | C015 | C24H40O5 | 407.280 | (−) | 0 |
| 16 | C017 | C27H52O4 | 439.379 | (−) | 0 |
| 17 | C031 | C34H43N3O3 | 540.331 | (−) | 14 |
| 18 | C033 | C33H34N4O6 | 581.241 | (−) | 1 |
| 19 | C120 | C21H38O4 | 355.283 | (+) | 4 |
| 20 | C132 | C23H21N5O5S | 480.134 | (+) | 1 |
| 21 | C145 | C23H46NO6P | 464.314 | (+) | 2 |
| 22 | DS01 | CA (Cholic Acid) | 407.281 | (−) | 0 |
| 23 | DS10 | UDCA | 391.286 | (−) | 0 |
| | | (Ursodeoxycholic Acid) | | | |
| 24 | DS11 | 5β-CAA-3β, 12α-2K | \ | | 0 |
| 25 | X006 | C13H16N2O4 | 263.104 | (−) | 1 |
| 26 | X011 | C18H32O4 | 311.223 | (−) | 1 |
| 27 | X016 | C20H32O3 | 319.228 | (−) | 0 |
| 28 | X023 | C19H28O5S | 367.158 | (−) | 1 |
| 29 | X036 | C23H27FN4O3 | 425.201 | (−) | 4 |
| 30 | X055 | C27H46NO7P | 526.315 | (−) | 40 |
| 31 | X066 | C29H56NO9P | 592.362 | (−) | 0 |
| 32 | X166 | C16H34NO5P | 352.224 | (+) | 2 |
| 33 | X278 | C11H18N2O7 | 289.106 | (−) | 6 |
| 34 | X280 | C30H40O3 | 447.312 | (−) | 48 |
| 35 | X281 | C27H44O5 | 447.312 | (−) | 1 |
| 36 | X285 | C26H43NO7S | 512.336 | (−) | 131 |
| 37 | X401 | C12H17NO3 | 222.114 | (−) | 2 |
| 38 | X407 | C17H17NO5 | 314.103 | (−) | 1 |
| 39 | X408 | C20H30O3 | 317.212 | (−) | 1 |
| 40 | X409 | C20H32O4 | 335.259 | (−) | 108 |
| 41 | X411 | C22H34O3 | 345.243 | (−) | 2 |
| 42 | X513 | C20H32O2 | 305.247 | (+) | 2 |
| 43 | X666 | C24H30O8 | 445.19 | (−) | 8 |
| 44 | X679 | C17H37NO2 | 288.289 | (+) | 2 |
| 45 | X665 | C26H44O4 | 419.316 | (−) | 1 |
| 46 | X659 | C18H32O3 | 295.228 | (−) | 1 |
| 47 | X667 | C26H46O6 | 453.321 | (−) | 2 |
| 48 | X660 | C17H26N4O | 301.202 | (−) | 3 |
| 49 | X657 | C8H7NO4S | 212.003 | (−) | 1 |
| 50 | X661 | C14H17NO8 | 327.099 | (−) | 10 |
| 51 | X662 | C21H31F3O | 355.228 | (−) | 7 |
| 52 | X663 | C22H26O6 | 385.169 | (−) | 9 |

In some embodiments, the one or more target metabolites for detecting CD may include one or more metabolite combinations shown in Table 10.

TABLE 10

| Meta ID | AUC | Sens | Spec |
|---|---|---|---|
| C146, X508 | 0.92 | 0.88 | 0.97 |
| C036, X508 | 0.91 | 0.89 | 0.96 |
| C019, C150 | 0.86 | 0.81 | 0.93 |
| C004, C027 | 0.93 | 0.83 | 0.97 |
| C019, X004 | 0.84 | 0.83 | 0.87 |
| C009, DS02 | 0.87 | 0.83 | 0.88 |
| C027, C147 | 0.88 | 0.86 | 0.84 |
| BP012, C019 | 0.85 | 0.79 | 0.88 |
| C146, C150, X508 | 0.93 | 0.9 | 0.93 |
| BN001, C150, X004 | 0.86 | 0.82 | 0.87 |
| BN001, C036, X508 | 0.92 | 0.9 | 0.95 |
| C019, C036, C150 | 0.86 | 0.8 | 0.92 |
| BN001, C004, C027 | 0.96 | 0.91 | 0.95 |
| C019, X004, X403 | 0.83 | 0.85 | 0.84 |
| C009, C027, DS02 | 0.9 | 0.87 | 0.89 |
| BP012, C027, C150 | 0.84 | 0.84 | 0.85 |
| BP012, C019, DS02 | 0.95 | 0.9 | 1 |

In some embodiments, the one or more target metabolites for detecting CD may include the one or more metabolic combinations shown in Table 10 but exclude any metabolites shown in Table 6. Alternatively, the one or more target metabolites may include the one or more metabolic combinations shown in Table 10 and at least one metabolite selected from the metabolites in Tables 6-10.

A method of detecting CD in a subject is provided. In some embodiments, the method may include: (a) obtaining, from a quantitative measurement device, quantified abundance of one or more target metabolites in a panel of metabolites in a sample from the subject; (b) determining a sample score by processing the quantified abundance of each of the one or more target metabolites using a prediction model; and (c) estimating whether the subject has CD by comparing the sample score to a cut-off score. The description of the one or more target metabolites may be found earlier in the present disclosure. For example, the one or more target metabolites may include at least one metabolite in the metabolites of Table 6. As another example, the one or more target metabolites may include at least one metabolite in the metabolites of Table 6, at least one metabolite selected from the metabolites of Table 7-10, and/or one or more metabolic combinations in Table 10.

In some embodiments, the method of detecting CD may be followed by a treatment for CD. For example, the treatment may include a surgery for removing a diseased bowel part and/or administering anti-CD therapeutics to the subject. Specifically, the treatment for CD may include anticholinergic agents and bile acid sequestrants, if there is no bowel obstruction. Additionally, several recently developed biologic medications have only been approved to treat CD.

More descriptions regarding the performance of some exemplary prediction models for detecting CD may be found in the Examples section.

According to another aspect of the present disclosure, a panel of metabolites for detecting whether a subject has UC is provided. In some embodiments, the group of metabolites may include one or more target metabolites correlated with UC. The one or more target metabolites may be serum metabolites that exhibit significant differentiation between a positive group of subjects (UC patients) and a negative group of subjects (normal people). More details regarding the determination of the one or more target metabolites may be found elsewhere in the present disclosure, e.g., Example 1.

Table 11 shows an exemplary group of metabolites that can be used for detecting UC. Each of the metabolites, which are biomarkers, has shown a strong and reliable correlation with the presence of UC. In some embodiments, the group of metabolites provided by the present disclosure may include one or more target metabolites of Table 11. In some embodiments, the group of metabolites may include at least one of the metabolites of Table 11. In some embodiments, the group of metabolites may include at least two of the metabolites of Table 11. In some embodiments, the group of metabolites may include at least 3, 4, or 5 of the metabolites of Table 11. As another example, the group of metabolites may include all of the metabolites of Table 11.

TABLE 11

| | | | | | Delta |
|---|---|---|---|---|---|
| No. | Meta ID | Compound | MASS | (+/−) | (ppm) |
| 1 | BN029 | Azelaic acid | 187.098 | (−) | 0 |
| 2 | C004 | C10H12N4O5 | 267.073 | (−) | 2 |
| 3 | C019 | C27H44O5 | 447.312 | (−) | 1 |
| 4 | C027 | C31H46O4 | 481.354 | (−) | 45 |
| 5 | C146 | C23H48NO7P | 482.324 | (+) | 0 |
| 6 | DS04 | GCA (Glycocholic Acid | 464.302 | (−) | 0 |
| | | Hydrate) | | | |
| 7 | X004 | C12H16O5 | 239.092 | (−) | 2 |
| 8 | X403 | C13H12N4O | 239.092 | (−) | 8 |
| 9 | X508 | C7H2F5NO | 212.020 | (+) | 33 |

In some embodiments, the one or more target metabolites for detecting UC and/or facilitating the treatment of UC may include at least one metabolite of Table 11 and at least one metabolite of Table 12. Each of the metabolites in Table 12 is found to be closely correlated with the presence of UC. In some embodiments, the one or more target metabolites may further include 1, 2, 3, or all of the metabolites of the metabolites in Table 12. For example, the one or more target metabolites may include one metabolite in Table 11 and one metabolite in Table 12. As another example, the one or more target metabolites may include one metabolite in Table A and two metabolites in Table 12. As yet another example, the one or more target metabolites may include two metabolites in Table 11 and one metabolite in Table 12. See, e.g., Example 2. Similarly, any combinations of one or more metabolites in Table 11 and one or more metabolites in Table 12 may be used to achieve the same purposes. In some embodiments, one or more of the metabolites in Table 12 may be used, independently from the metabolites listed in Table 11, for detecting and/or facilitating the treatment of UC in the subject.

TABLE 12

| | | | | | Delta |
|---|---|---|---|---|---|
| No. | Meta ID | Compound | MASS (+/−) | | (ppm) |
| 1 | X082 | C19H26N6O | 353.212 | (−) | 7 |
| 2 | C009 | C22H20N4O | 355.158 | (−) | 4 |
| 3 | BN001 | 5_HETE | 319.228 | (−) | 0 |
| 4 | C036 | C31H57N5)9 | 642.396 | (−) | 19 |
| 5 | C147 | C25H47NO9 | 506.323 | (+) | 18 |
| 6 | BP012 | (±)-Hexanoyl carnitine | 261.193 | (+) | 0 |
| | | chloride | | | |
| 7 | C150 | C27H48NO7P | 530.324 | (+) | 1 |
| 8 | DS02 | CDCA | 391.286 | (−) | 0 |
| | | (Chenodeoxycholic | | | |
| | | Acid) | | | |

TABLE 13
In some embodiments, one or more of the
metabolites in Table 13 may be used,

| | | | | Delta |
|---|---|---|---|---|
| No. | Meta ID | Compound | MASS (+/−) | (ppm) |
| 1 | DS03 | DCA (Deoxycholic | 391.286(−) | 0 |
| | | Acid) | | |

in addition to the one or more metabolites listed in Table 11 and/or one or more metabolites listed in Table 12, for detecting UC and/or facilitating the treatment of UC in the subject. In some embodiments, the abundance of the metabolite of the metabolites in Table 13 may be quantified for the same purposes.

In some embodiments, the one or more target metabolites provided by the present disclosure may include at least one metabolite of Table 14. Each of the metabolites in Table 14 is found to be correlated with the presence of UC. In some embodiments, one or more of the metabolites in Table 14 may be used, in addition to the one or more metabolites listed in Table 11 and/or one or more metabolites listed in Table 12, for detecting UC and/or facilitating the treatment of UC in the subject. As another example, one or more of the metabolites in Table 14 may be used, in addition to the one or more metabolites listed in Table 11, one or more metabolites listed in Table 12, and one or more metabolites listed in Table 13 for the same purposes. In some embodiments, the abundance of 1, 2, 3 or all the metabolites of the metabolites in Table 14 may be quantified.

TABLE 14

| | | | | | Delta |
|---|---|---|---|---|---|
| No. | Meta ID | Compound | MASS (+/−) | | (ppm) |
| 1 | BN023 | Eicosapentaenoic Acid | 301.218 | (−) | 0 |
| 2 | BN025 | Hydrocortisone | 361.202 | (−) | 0 |
| 3 | BN028 | Pimelic acid | 159.067 | (−) | 0 |
| 4 | BP007 | trans-2-octenoyl-I- | 286.201 | (+) | 0 |
| | | carnitine | | | |
| 5 | BP010 | trans-2-Hexadecenoyl- | 398.326 | (+) | 0 |
| | | L-carnitine | | | |
| 6 | BP011 | Decanoyl-L-carnitine | 316.248 | (+) | 0 |
| 7 | BP013 | 17-Alpha- | 303.232 | (+) | 0 |
| | | Methyltestosterone | | | |
| 8 | C011 | C16H30O10 | 381.174 | (−) | 7 |
| 9 | C012 | C19H21N5O3S | 398.132 | (−) | 7 |
| 10 | C017 | C27H52O4 | 439.379 | (−) | 0 |
| 11 | C021 | C29H43NO2S | 468.308 | (−) | 30 |
| 12 | C026 | C25H43N3O6 | 480.310 | (−) | 4 |
| 13 | C031 | C34H43N3O3 | 540.331 | (−) | 14 |
| 14 | C035 | C33H45N5O5 | 590.346 | (−) | 19 |
| 15 | C043 | C31H44O6 | 511.302 | (−) | 9 |
| 16 | C102 | C7H8N4O2 | 181.072 | (+) | 1 |
| 17 | C110 | C15H27NO4 | 286.201 | (+) | 1 |
| 18 | C114 | C19H37NO6 | 317.195 | (+) | 3 |
| 19 | C122 | C24H37NO2 | 372.300 | (+) | 28 |
| 20 | C124 | C23H43NO4 | 398.325 | (+) | 4 |
| 21 | C129 | C25H47NO5 | 442.352 | (+) | 2 |
| 22 | C131 | C22H46NO7P | 468.308 | (+) | 1 |
| 23 | C132 | C23H21N5O5S | 480.134 | (+) | 1 |
| 24 | C135 | C8H10N4O2 | 195.087 | (+) | 2 |
| 25 | C136 | C19H26O2 | 287.204 | (+) | 14 |
| 26 | C145 | C23H46NO6P | 464.314 | (+) | 2 |
| 27 | C148 | C25H50NO7P | 508.340 | (+) | 1 |
| 28 | C149 | C28H45NO7 | 508.340 | (+) | 26 |
| 29 | DS05 | GCDCA | 448.307 | (−) | 0 |
| | | (Glycochenodeoxycholic | | | |
| | | Acid) | | | |
| 30 | DS07 | GLCA (Glycolithocholic | 432.312 | (−) | 0 |
| | | Acid) | | | |
| 31 | DS10 | UDCA (Ursodeoxycholic | 391.286 | (−) | 0 |
| | | Acid) | | | |
| 32 | X006 | C13H16N2O4 | 263.104 | (−) | 1 |
| 33 | X013 | C18H34O4 | 313.238 | (−) | 1 |
| 34 | X023 | C19H28O5S | 367.158 | (−) | 1 |
| 35 | X055 | C27H46NO7P | 526.315 | (−) | 40 |
| 36 | X066 | C29H56NO9P | 592.362 | (−) | 0 |
| 37 | X070 | C34H66NO8P | 646.427 | (−) | 28 |
| 38 | X160 | C17H33NO4 | 316.247 | (+) | 4 |
| 39 | X166 | C16H34NO5P | 352.224 | (+) | 2 |
| 40 | X183 | C27H41F2N5O | 490.300 | (+) | 72 |
| 41 | X278 | C11H18N2O7 | 289.106 | (−) | 6 |
| 42 | X281 | C27H44O5 | 447.312 | (−) | 1 |
| 43 | X285 | C26H43NO7S | 512.336 | (−) | 131 |
| 44 | X286 | C29H41F2N5O | 512.336 | (−) | 30 |
| 45 | X289 | C29H47NO8 | 536.299 | (−) | 45 |
| 46 | X401 | C12H17NO3 | 222.114 | (−) | 2 |
| 47 | X408 | C20H30O3 | 317.212 | (−) | 1 |
| 48 | X409 | C20H32O4 | 335.259 | (−) | 108 |
| 49 | X411 | C22H34O3 | 345.243 | (−) | 2 |
| 50 | X412 | C19H22N4O3 | 353.164 | (−) | 6 |
| 51 | X515 | C19H18N4O2 | 335.151 | (+) | 2 |
| 52 | X655 | C10H18O4 | 201.114 | (−) | 3 |
| 53 | X667 | C26H46O6 | 453.321 | (−) | 2 |
| 54 | X650 | C8H9NO2 | 150.056 | (−) | 5 |
| 55 | X653 | C9H9NO4 | 194.046 | (−) | 1 |
| 56 | X683 | C22H24N2O5 | 397.183 | (+) | 17 |
| 57 | X652 | C9H9NO4 | 194.046 | (−) | 1 |
| 58 | X680 | C20H30O2 | 303.231 | (+) | 2 |
| 59 | X651 | C6H6O5S | 188.987 | (−) | 1 |
| 60 | X654 | C10H18O4 | 201.114 | (−) | 6 |
| 61 | X656 | C4H8NO7P | 212.003 | (−) | 28 |

In some embodiments, the one or more target metabolites for detecting UC may include one or more metabolite combinations shown in Table 15.

TABLE 15

| Meta ID | AUC | Sens | Spec |
|---|---|---|---|
| C147, X082 | 0.83 | 0.82 | 0.8 |
| BP012, C147 | 0.84 | 0.83 | 0.82 |
| C019, C150 | 0.84 | 0.86 | 0.82 |
| C009, DS02 | 0.83 | 0.86 | 0.78 |
| BP012, C009, C147 | 0.84 | 0.84 | 0.8 |
| BN001, C147, X082 | 0.84 | 0.83 | 0.8 |
| BP012, C009C147 | 0.84 | 0.84 | 0.8 |

In some embodiments, the one or more target metabolites for detecting UC may include the one or more metabolic combinations shown in Table 15 but exclude any metabolites shown in Table 11. Alternatively, the one or more target metabolites may include the one or more metabolic combinations shown in Table 15 and at least one metabolite selected from the metabolites in Table s 11-15.

A method of detecting UC in a subject is provided. In some embodiments, the method may include: (a) obtaining, from a quantitative measurement device, quantified abundance of one or more target metabolites in a panel of metabolites in a sample from the subject; (b) determining a sample score by processing the quantified abundance of each of the one or more target metabolites using a prediction model; and (c) estimating whether the subject has UC by comparing the sample score to a cut-off score. The description of the one or more target metabolites may be found earlier in the present disclosure. For example, the one or more target metabolites may include at least one metabolite in the metabolites of Table 11. As another example, the one or more target metabolites may include at least one metabolite in the metabolites of Table 11, at least one metabolite selected from the metabolites of Table 12-15, and/or one or more metabolic combinations in Table 15.

In some embodiments, the method of detecting UC may be followed by a treatment for UC. For example, the treatment may include a surgery for removing a diseased bowel part and/or administering anti-UC therapeutics to the subject. Specifically, the treatment of UC may include 5-ASA medications, Colazal (balsalazide disodium).

More descriptions regarding the performance of some exemplary prediction models for detecting UC may be found in the Examples section.

According to another aspect of the present disclosure, a panel of metabolites for determining whether a subject has CD or UC is provided. In some embodiments, the group of metabolites may include one or more target metabolites correlated with CD and UC. The one or more target metabolites may be serum metabolites that exhibit significant differentiation between subjects having CD and subjects having UC. More details regarding the determination of the one or more target metabolites may be found elsewhere in the present disclosure, e.g., Example 1.

In some embodiments, the panel of metabolites for detecting IBD in a subject may be firstly used to determine whether the subject has IBD. If the subject has IBD, the panel of metabolites for determining whether the subject has CD or UC may be used to distinguish the subtype of IBD for the subject.

Table 16 shows an exemplary group of metabolites that can be used for determining whether a subject has CD or UC. Each of the metabolites, which are biomarkers, has shown a strong and reliable correlation with the presence of UC and CD. In some embodiments, the group of metabolites provided by the present disclosure may include one or more target metabolites of Table 16. In some embodiments, the group of metabolites may include at least one of the metabolites of Table 16. In some embodiments, the group of metabolites may include at least two of the metabolites of Table 16. In some embodiments, the group of metabolites may include at least 1, 2, or 3 of the metabolites of Table 16. As another example, the group of metabolites may include all of the metabolites of Table 16.

TABLE 16

| | | | | Delta |
|---|---|---|---|---|
| No. | Meta ID | Compound | MASS (+/−) | (ppm) |
| 1 | DS11 | 5β-CAA-3β, 12α-2K | \ | 0 |
| 2 | X082 | C19H26N6O | 353.212 (−) | 7 |
| 3 | C128 | C28H43O3 | 428.363 (+) | 79 |

In some embodiments, the one or more target metabolites for determining whether a subject has CD or UC and/or facilitating the treatment of CD or UC may include at least one metabolite of Table 16 and at least one metabolite of Table 17. In some embodiments, the one or more target metabolites may further include 1, 2, 3, or all of the metabolites of the metabolites in Table 17. For example, the one or more target metabolites may include one metabolite in Table 16 and one metabolite in Table 17. As another example, the one or more target metabolites may include one metabolite in Table A and two metabolites in Table 17. As yet another example, the one or more target metabolites may include two metabolites in Table 16 and one metabolite in Table 17. See, e.g., Example 2. Similarly, any combinations of one or more metabolites in Table 16 and one or more metabolites in Table 17 may be used to achieve the same purposes.

TABLE 17

| | | | | Delta |
|---|---|---|---|---|
| No. | Meta ID | Compound | MASS (+/−) | (ppm) |
| 1 | BN012 | 14(S)-HDHA | 343.228(−) | 0 |
| 2 | BN025 | Hydrocortisone | 361.202(−) | 0 |

In some embodiments, one or more of the metabolites in Table 18 may be used, in addition to the one or more metabolites listed in Table 16 and/or one or more metabolites listed in Table 17, for determining whether a subject has CD or UC and/or facilitating the treatment of CD/UC in the subject. In some embodiments, the abundance of 1, 2, 3 or all the metabolites of the metabolites in Table 18 may be quantified for the same purposes.

TABLE 18

| | | | | | Delta |
|---|---|---|---|---|---|
| No. | Meta ID | Compound | MASS (+/−) | | (ppm) |
| 1 | DS05 | GCDCA | 448.307 | (−) | 0 |
| | | (Glycochenodeoxycholic | | | |
| | | Acid) | | | |
| 2 | DS04 | GCA (Glycocholic Acid | 464.302 | (−) | 0 |
| | | Hydrate) | | | |
| 3 | DS07 | GLCA (Glycolithocholic | 432.312 | (−) | 0 |
| | | Acid) | | | |
| 4 | C008 | C19H36O4 | 327.256 | (−) | 5 |
| 5 | C112 | C15H17F3N2O2 | 315.134 | (+) | 9 |
| 6 | C146 | C23H48NO7P | 482.324 | (+) | 0 |
| 7 | X515 | C19H18N4O2 | 335.151 | (+) | 2 |
| 8 | X407 | C17H17NO5 | 314.103 | (−) | 1 |
| 9 | X411 | C22H34O3 | 345.243 | (−) | 2 |
| 10 | BP003 | 2-Arachidonoyl Glycerol | 379.284 | (+) | 0 |

In some embodiments, the one or more target metabolites provided by the present disclosure may include at least one metabolite of Table 19. In some embodiments, one or more of the metabolites in Table 19 may be used, in addition to the one or more metabolites listed in Table 16 and/or one or more metabolites listed in Table 17, for determining whether a subject has CD or UC and/or facilitating the treatment of CD/UC in the subject. As another example, one or more of the metabolites in Table 19 may be used, in addition to the one or more metabolites listed in Table 16, one or more metabolites listed in Table 17, and one or more metabolites listed in Table 18 for the same purposes. In some embodiments, the abundance of 1, 2, 3 or all the metabolites of the metabolites in Table 19 may be quantified.

TABLE 19

| | | | | | Delta |
|---|---|---|---|---|---|
| No. | Meta ID | Compound | MASS (+/−) | | (ppm) |
| 1 | BN030 | Arachidonic acid | 303.233 | (−) | 0 |
| 2 | BP006 | Myristoyl-L-carnitine | 372.311 | (+) | 0 |
| 3 | BP010 | trans-2-Hexadecenoyl- | 398.326 | (+) | 0 |
| | | L-carnitine | | | |
| 4 | BP012 | (±)-Hexanoyl carnitine | 261.193 | (+) | 0 |
| | | chloride | | | |
| 5 | C006 | C18H32O3 | 295.229 | (−) | 2 |
| 6 | C009 | C22H20N4O | 355.158 | (−) | 4 |
| 7 | C011 | C16H30O10 | 381.174 | (−) | 7 |
| 8 | C043 | C31H44O6 | 511.302 | (−) | 9 |
| 9 | C122 | C24H37NO2 | 372.300 | (+) | 28 |
| 10 | C124 | C23H43NO4 | 398.325 | (+) | 4 |
| 11 | C129 | C25H47NO5 | 442.352 | (+) | 2 |
| 12 | X055 | C27H46NO7P | 526.315 | (−) | 40 |
| 13 | X292 | C7H8O4S | 187.007 | (−) | 0 |
| 14 | X412 | C19H22N4O3 | 353.164 | (−) | 6 |
| 15 | X676 | C9H18N4O4 | 247.144 | (+) | 15 |
| 16 | X653 | C9H9NO4 | 194.046 | (−) | 1 |
| 17 | X659 | C18H32O3 | 295.228 | (−) | 1 |
| 18 | X665 | C26H44O4 | 419.316 | (−) | 1 |
| 19 | X652 | C9H9NO4 | 194.046 | (−) | 1 |
| 20 | X658 | C15H12O5 | 271.05 | (−) | 26 |
| 21 | X673 | C9H12O3 | 169.086 | (+) | 1 |
| 22 | X670 | C32H54O5 | 517.39 | (−) | 1 |
| 23 | X672 | C9H7N | 130.065 | (+) | 1 |
| 24 | X675 | C6H14NO2Se | 212.02 | (+) | 6 |

In some embodiments, the one or more target metabolites for determining whether a subject has CD or UC may include one or more metabolite combinations shown in Table 20.

TABLE 20

| Meta ID | AUC | Sens | Spec |
|---|---|---|---|
| BN025, C008, DS04 | 0.76 | 0.61 | 0.86 |
| C112, C146, X515 | 0.76 | 0.57 | 0.9 |
| BN025, DS07, X082 | 0.78 | 0.69 | 0.84 |
| C008, DS04, DS07 | 0.77 | 0.59 | 0.88 |

In some embodiments, the one or more target metabolites for determining whether a subject has CD or UC may include the one or more metabolic combinations shown in Table 20 but exclude any metabolites shown in Table 16. Alternatively, the one or more target metabolites may include the one or more metabolic combinations shown in Table 20 and at least one metabolite selected from the metabolites in Tables 16-19.

A method of determining whether a subject has CD or UC in a subject is provided. In some embodiments, the method may include: (a) obtaining, from a quantitative measurement device, quantified abundance of one or more target metabolites in a panel of metabolites in a sample from the subject; (b) determining a sample score by processing the quantified abundance of each of the one or more target metabolites using a prediction model; and (c) estimating whether the subject has UC or CD by comparing the sample score to a cut-off score. The description of the one or more target metabolites may be found earlier in the present disclosure. For example, the one or more target metabolites may include at least one metabolite in the metabolites of Table 16. As another example, the one or more target metabolites may include at least one metabolite in the metabolites of Table 16, at least one metabolite selected from the metabolites of Table 17-20, and/or one or more metabolic combinations in Table 20.

In some embodiments, the method of determining whether the subject has CD or UC in a subject may be followed by a treatment accordingly. For example, the treatment may include a surgery for removing a diseased bowel part and/or administering anti-UC therapeutics/anti-CD therapeutics to the subject accordingly.

More descriptions regarding the performance of some exemplary prediction models for determining whether the subject has CD or UC may be found in the Examples section.

According to another aspect of the present disclosure, a panel of metabolites for determining whether a subject has IBD or colorectal polyp is provided. As used herein, the term “colorectal polyp” refers to colorectal adenoma and non-adenoma polyps. In some embodiments, the group of metabolites may include one or more target metabolites correlated with IBD. The one or more target metabolites may be serum metabolites that exhibit significant differentiation between subjects having IBD and subjects having colorectal polyp. More details regarding the determination of the one or more target metabolites may be found elsewhere in the present disclosure, e.g., Example 1.

Conventional methods for discriminating between IBD and colorectal polyp use the combination of colonoscopy examination and histological examination of the biopsies. The approach for determining whether a subject has IBD or colorectal polyp provided by the present disclosure is a non-invasive method utilizing the panel of specific metabolites, which may reduce the pain and the hurt to the patient.

Table 21 shows an exemplary group of metabolites that can be used for determining whether a subject has IBD or colorectal polyp. Each of the metabolites, which are biomarkers, has shown a strong and reliable correlation with the presence of IBD. In some embodiments, the group of metabolites provided by the present disclosure may include one or more target metabolites of Table 21. In some embodiments, the group of metabolites may include at least one of the metabolites of Table 21. In some embodiments, the group of metabolites may include at least two of the metabolites of Table 21. In some embodiments, the group of metabolites may include at least 3, 4, or 5 of the metabolites of Table 21. As another example, the group of metabolites may include all of the metabolites of Table 21.

TABLE 21

| | | | | | Delta |
|---|---|---|---|---|---|
| No. | Meta ID | Compound | MASS (+/−) | | (ppm) |
| 1 | BN029 | Azelaic acid | 187.098 | (−) | 0 |
| 2 | C004 | C10H12N4O5 | 267.073 | (−) | 2 |
| 3 | C017 | C27H52O4 | 439.379 | (−) | 0 |
| 4 | C027 | C31H46O4 | 481.354 | (−) | 45 |
| 5 | C102 | C7H8N4O2 | 181.072 | (+) | 1 |
| 6 | X403 | C13H12N4O | 239.092 | (−) | 8 |
| 7 | X408 | C20H30O3 | 317.212 | (−) | 1 |
| 8 | X411 | C22H34O3 | 345.243 | (−) | 2 |
| 9 | X508 | C7H2F5NO | 212.020 | (+) | 33 |

In some embodiments, the one or more target metabolites for determining whether a subject has IBD or colorectal polyp and/or facilitating the treatment of IBD/colorectal polyp may include at least one metabolite of Table 21 and at least one metabolite of Table 22. Each of the metabolites in Table 22 is found to be closely correlated with the presence of IBD. In some embodiments, the one or more target metabolites may further include 1, 2, 3, or all of the metabolites of the metabolites in Table 22. For example, the one or more target metabolites may include one metabolite in Table 21 and one metabolite in Table 22. As another example, the one or more target metabolites may include one metabolite in Table A and two metabolites in Table 2. As yet another example, the one or more target metabolites may include two metabolites in Table 21 and one metabolite in Table 22. See, e.g., Example 2. Similarly, any combinations of one or more metabolites in Table 21 and one or more metabolites in Table 22 may be used to achieve the same purposes. In some embodiments, one or more of the metabolites in Table 22 may be used, independently from the metabolites listed in Table 21, for detecting and/or facilitating the treatment of IBD/colorectal polyp in the subject.

TABLE 22

| | Meta | | | | Delta |
|---|---|---|---|---|---|
| No. | ID | Compound | MASS (+/−) | | (ppm) |
| 1 | X293 | C11H11NO3 | 204.067 | (−) | 2 |
| 2 | BN035 | 9(S),10(S),13(S)_Trihydroxy_11(E)_Octadecenoic | 329.234 | (−) | 0 |
| | | Acid | | | |
| 3 | C035 | C33H45N5O5 | 590.346 | (−) | 19 |
| 4 | X082 | C19H26N6O | 353.212 | (−) | 7 |
| 5 | C112 | C15H17F3N2O2 | 315.134 | (+) | 9 |
| 6 | DS02 | CDCA (Chenodeoxycholic Acid) | 391.286 | (−) | 0 |
| 7 | DS04 | GCA (Glycocholic Acid Hydrate) | 464.302 | (−) | 0 |

In some embodiments, one or more of the metabolites in Table 23 may be used, in addition to the one or more metabolites listed in Table 21 and/or one or more metabolites listed in Table 22, for determining whether a subject has IBD or colorectal polyp and/or facilitating the treatment of IBD in the subject. In some embodiments, the abundance of 1, 2, 3 or all the metabolites of the metabolites in Table 23 may be quantified for the same purposes.

TABLE 23

| | | | | | Delta |
|---|---|---|---|---|---|
| No. | Meta ID | Compound | MASS (+/−) | | (ppm) |
| 1 | X024 | C19H30O5S | 369.174 | (−) | 0 |
| 2 | BN021 | 3-Dehydrocholic Acid | 405.265 | (−) | 0 |
| 3 | BP002 | S-(−)-Cotinine | 177.102 | (+) | 0 |
| 4 | C021 | C29H43NO2S | 468.308 | (−) | 30 |

In some embodiments, the one or more target metabolites provided by the present disclosure may include at least one metabolite of Table 24. Each of the metabolites in Table 24 is found to be correlated with the presence of IBD. In some embodiments, one or more of the metabolites in Table 24 may be used, in addition to the one or more metabolites listed in Table 21 and/or one or more metabolites listed in Table 22, for determining whether a subject has IBD or colorectal polyp and/or facilitating the treatment of IBD in the subject. As another example, one or more of the metabolites in Table 24 may be used, in addition to the one or more metabolites listed in Table 21, one or more metabolites listed in Table 22, and one or more metabolites listed in Table 23 for the same purposes. In some embodiments, the abundance of 1, 2, 3 or all the metabolites of the metabolites in Table 24 may be quantified.

TABLE 24

| | | | | | Delta |
|---|---|---|---|---|---|
| No. | Meta ID | Compound | MASS (+/−) | | (ppm) |
| 1 | BN001 | 5_HETE | 319.228 | (−) | 0 |
| 2 | BN003 | 8_HETE | 319.228 | (−) | 0 |
| 3 | BN006 | 12_HETE | 319.228 | (−) | 0 |
| 4 | BN012 | 14(S)-HDHA | 343.228 | (−) | 0 |
| 5 | BN015 | Sphingosine-1-phosphate (d16:1) | 350.210 | (−) | 0 |
| 6 | BN016 | Octadecane dioic acid | 313.239 | (−) | 0 |
| 7 | BN017 | Epitestosterone Sulfate | \ | | 0 |
| 8 | BN020 | 3α-Hydroxy-6-OXO-5α-Cholan-24-OIC | 389.270 | (−) | 0 |
| | | Acid | | | |
| 9 | BN022 | 5α-Cholanic Acid-3α, 7β-Diol-6-One | 405.265 | (−) | 0 |
| 10 | BN023 | Eicosapentaenoic Acid | 301.218 | (−) | 0 |
| 11 | BN027 | 1-Stearoyl-2-Hydroxy-sn-Glycero-3- | 480.310 | (−) | 0 |
| | | Phosphoethanolamine | | | |
| 12 | BN028 | Pimelic acid | 159.067 | (−) | 0 |
| 13 | BN030 | Arachidonic acid | 303.233 | (−) | 0 |
| 14 | BP003 | 2-Arachidonoyl Glycerol | 379.284 | (+) | 0 |
| 15 | BP007 | trans-2-octenoyl-I-carnitine | 286.201 | (+) | 0 |
| 16 | BP009 | 1-Linoleoyl-rac-glycerol | 355.284 | (+) | 0 |
| 17 | BP013 | 17-Alpha-Methyltestosterone | 303.232 | (+) | 0 |
| 18 | C008 | C19H36O4 | 327.256 | (−) | 5 |
| 19 | C016 | C24H23F3N2O2 | 427.163 | (−) | 2 |
| 20 | C019 | C27H44O5 | 447.312 | (−) | 1 |
| 21 | C026 | C25H43N3O6 | 480.310 | (−) | 4 |
| 22 | C031 | C34H43N3O3 | 540.331 | (−) | 14 |
| 23 | C032 | C33H35N5O5 | 580.235 | (−) | 37 |
| 24 | C033 | C33H34N4O6 | 581.241 | (−) | 1 |
| 25 | C041 | C26H44O9 | 499.288 | (−) | 7 |
| 26 | C043 | C31H44O6 | 511.302 | (−) | 9 |
| 27 | C110 | C15H27NO4 | 286.201 | (+) | 1 |
| 28 | C116 | C18H35NO4 | 330.263 | (+) | 2 |
| 29 | C120 | C21H38O4 | 355.283 | (+) | 4 |
| 30 | C131 | C22H46NO7P | 468.308 | (+) | 1 |
| 31 | C132 | C23H21N5O5S | 480.134 | (+) | 1 |
| 32 | C135 | C8H10N4O2 | 195.087 | (+) | 2 |
| 33 | C136 | C19H26O2 | 287.204 | (+) | 14 |
| 34 | C137 | C19H27NO2 | 302.215 | (+) | 11 |
| 35 | C139 | C21H40O3 | 341.306 | (+) | 2 |
| 36 | C144 | C24H36O2 | 357.280 | (+) | 3 |
| 37 | C145 | C23H46NO6P | 464.314 | (+) | 2 |
| 38 | C146 | C23H48NO7P | 482.324 | (+) | 0 |
| 39 | C147 | C25H47NO9 | 506.323 | (+) | 18 |
| 40 | C148 | C25H50NO7P | 508.340 | (+) | 1 |
| 41 | C149 | C28H45NO7 | 508.340 | (+) | 26 |
| 42 | C150 | C27H48NO7P | 530.324 | (+) | 1 |
| 43 | DS01 | CA (Cholic Acid) | 407.281 | (−) | 0 |
| 44 | DS05 | GCDCA (Glycochenodeoxycholic | 448.307 | (−) | 0 |
| | | Acid) | | | |
| 45 | DS10 | UDCA (Ursodeoxycholic Acid) | 391.286 | (−) | 0 |
| 46 | DS11 | 5β-CAA-3β, 12α-2K | \ | | 0 |
| 47 | X004 | C12H16O5 | 239.092 | (−) | 2 |
| 48 | X006 | C13H16N2O4 | 263.104 | (−) | 1 |
| 49 | X013 | C18H34O4 | 313.238 | (−) | 1 |
| 50 | X016 | C20H32O3 | 319.228 | (−) | 0 |
| 51 | X023 | C19H28O5S | 367.158 | (−) | 1 |
| 52 | X032 | C25H24O5 | 403.158 | (−) | 7 |
| 53 | X055 | C27H46NO7P | 526.315 | (−) | 40 |
| 54 | X066 | C29H56NO9P | 592.362 | (−) | 0 |
| 55 | X154 | C16H23N5O | 302.196 | (+) | 5 |
| 56 | X166 | C16H34NO5P | 352.224 | (+) | 2 |
| 57 | X183 | C27H41F2N5O | 490.300 | (+) | 72 |
| 58 | X188 | C28H48NO7P | 542.324 | (+) | 0 |
| 59 | X278 | C11H18N2O7 | 289.106 | (−) | 6 |
| 60 | X280 | C30H40O3 | 447.312 | (−) | 48 |
| 61 | X281 | C27H44O5 | 447.312 | (−) | 1 |
| 62 | X285 | C26H43NO7S | 512.336 | (−) | 131 |
| 63 | X286 | C29H41F2N5O | 512.336 | (−) | 30 |
| 64 | X289 | C29H47NO8 | 536.299 | (−) | 45 |
| 65 | X292 | C7H8O4S | 187.007 | (−) | 0 |
| 66 | X401 | C12H17NO3 | 222.114 | (−) | 2 |
| 67 | X409 | C20H32O4 | 335.259 | (−) | 108 |
| 68 | X513 | C20H32O2 | 305.247 | (+) | 2 |
| 69 | X519 | C11H18N5O7P | 364.084 | (+) | 49 |

In some embodiments, the one or more target metabolites for determining whether a subject has IBD or colorectal polyp may include the metabolite combination shown in Table 25.

TABLE 25

| Meta ID | AUC | Sens | Spec |
|---|---|---|---|
| BP002, BN021, C021 | 0.81 | 0.82 | 0.75 |

In some embodiments, the one or more target metabolites for determining whether a subject has IBD or colorectal polyp may include the one or more metabolic combinations shown in Table 25 but exclude any metabolites shown in Table 21. Alternatively, the one or more target metabolites may include the one or more metabolic combinations shown in Table 25 and at least one metabolite selected from the metabolites in Tables 21-24.

A method of determining whether a subject has IBD or colorectal polyp in a subject is provided. In some embodiments, the method may include: (a) obtaining, from a quantitative measurement device, quantified abundance of one or more target metabolites in a panel of metabolites in a sample from the subject; (b) determining a sample score by processing the quantified abundance of each of the one or more target metabolites using a prediction model; and (c) estimating whether the subject has IBD or colorectal polyp by comparing the sample score to a cut-off score. The description of the one or more target metabolites may be found earlier in the present disclosure. For example, the one or more target metabolites may include at least one metabolite in the metabolites of Table 21. As another example, the one or more target metabolites may include at least one metabolite in the metabolites of Table 21, at least one metabolite selected from the metabolites of Table 22-25, and/or one or more metabolic combinations in Table 25.

In some embodiments, the method of determining whether the subject has IBD or colorectal polyp is provided. The method may be followed by a treatment for IBD. For example, the treatment may include a surgery for removing the colorectal polyp and/or administering anti-IBD therapeutics to the subject.

More descriptions regarding the performance of some exemplary prediction models for determining whether a subject has IBD or colorectal polyp may be found in the Examples section.

According to another aspect of the present disclosure, a method of identifying gut microbiome-associated (GMA) metabolites as biomarkers for a prediction panel for IBD is provided. The method includes: obtaining source data by conducting untargeted mass spectrometry to samples from IBD patients and control group of persons not having IBD; identifying a first group of metabolites that are significantly altered in the IBD patients; identifying a second group of metabolites from the first group by selecting metabolites that show significant correlation with gut microbiome; and selecting the GMA metabolites for the prediction panel from the second group of metabolites using a selection model.

According to another aspect of the present disclosure, a method of identifying GMA metabolites as biomarkers for a prediction panel for CD is provided. The method includes: obtaining source data by conducting untargeted mass spectrometry to samples from CD patients and control group of persons not having CD; identifying a first group of metabolites that are significantly altered in the CD patients; identifying a second group of metabolites from the first group by selecting metabolites that show significant correlation with gut microbiome; and selecting the GMA metabolites for the prediction panel from the second group of metabolites using a selection model.

According to another aspect of the present disclosure, a method of identifying GMA metabolites as biomarkers for a prediction panel for UC is provided. The method includes: obtaining source data by conducting untargeted mass spectrometry to samples from UC patients and control group of persons not having UC; identifying a first group of metabolites that are significantly altered in the UC patients; identifying a second group of metabolites from the first group by selecting metabolites that show significant correlation with gut microbiome; and selecting the GMA metabolites for the prediction panel from the second group of metabolites using a selection model.

According to another aspect of the present disclosure, a method of identifying gut microbiome-associated GMA metabolites as biomarkers for a prediction panel for determining whether a subject has Crohn's disease CD or Ulcerative colitis UC. The method includes: obtaining source data by conducting untargeted mass spectrometry to samples from CD patients and samples from UC patients; identifying a first group of metabolites that are significantly altered between the UC patients and the CD patients; identifying a second group of metabolites from the first group by selecting metabolites that show significant correlation with gut microbiome; and selecting the GMA metabolites for the prediction panel from the second group of metabolites using a selection model.

According to another aspect of the present disclosure, a method of identifying gut microbiome-associated GMA metabolites as biomarkers for a prediction panel for determining whether a subject has inflammatory bowel disease IBD or colorectal polyp is provided. The method includes: obtaining source data by conducting untargeted mass spectrometry to samples from IBD patients and samples from patients having colorectal polyps; identifying a first group of metabolites that are significantly altered between the IBD patients and the patients having colorectal polyps; identifying a second group of metabolites from the first group by selecting metabolites that show significant correlation with gut microbiome; and selecting the GMA metabolites for the prediction panel from the second group of metabolites using a selection model.

In some embodiments, a use of one or more target metabolites in generating a trained machine-learning model for determining whether a subject has IBD is provided. The one or more target metabolites include at least one, two, three, or all of metabolites in Table 1.

In some embodiments, a use of one or more target metabolites in generating a trained machine-learning model for determining whether a subject has CD. The one or more target metabolites include at least one, two, three, four, or five of metabolites in Table 6.

In some embodiments, a use of one or more target metabolites in generating a trained machine-learning model for determining whether a subject has UC is provided. The one or more target metabolites include at least one, two, three, or ten of metabolites in Table 11.

In some embodiments, a use of one or more target metabolites in generating a trained machine-learning model for determining whether a subject has CD or UC is provided.

In some embodiments, a use of one or more target metabolites in generating a trained machine-learning model for determining whether a subject has IBD or colorectal polyp. The one or more target metabolites include at least one, two, three, four, or all of metabolites in Table 21.

In some embodiments, a use of one or more target metabolites for preparing a kit for detecting IBD in a subject, the one or more target metabolites including at least one, two, three, or all of metabolites in Table 1.

In some embodiments, a use of one or more target metabolites for preparing a kit for detecting CD in a subject is provided. The one or more target metabolites may include at least one, two, three, or all of metabolites in Table 6.

In some embodiments, a use of one or more target metabolites for preparing a kit for detecting UC in a subject, the one or more target metabolites including at least o one, two, three, or all of metabolites in Table 11.

In some embodiments, a use of one or more target metabolites for preparing a kit for determining whether a subject has CD or UC is provided. The one or more target metabolites may include one, two, or all of metabolites in Table 16.

In some embodiments, a use of one or more target metabolites for preparing a kit for determining whether a subject has IBD or colorectal polyp is provided. The one or more target metabolites including at least one, two, three, or all of metabolites in Table 21.

In some embodiments, a kit for detecting IBD in a subject is provided. The kit may include one or more target metabolites, and the one or more target metabolites include at least one, two, three, or all of metabolites in Table 1.

In some embodiments, a kit for detecting CD in a subject is provided. The kit includes one or more target metabolites, and the one or more target metabolites include at least one, two, three, or all of metabolites in Table 6.

In some embodiments, a kit for detecting UC in a subject is provided. The kit may include one or more target metabolites, wherein the one or more target metabolites include at least one, two, three, four, five, or all of metabolites in Table 11.

In some embodiments, a kit for determining whether a subject has CD or UC in a subject is provided. The kit may include one or more target metabolites, wherein the one or more target metabolites include at least one, two, or all of metabolites in Table 16.

In some embodiments, a kit for determining whether a subject has IBD or colorectal polyp is provided. The kit may include one or more target metabolites, wherein the one or more target metabolites include at least one, two, three, or all of metabolites in Table 21.

The methods and metabolite biomarkers provided by the present disclosure are further described according to the following examples, which should not be construed as limiting the scope of the present disclosure. More description regarding the performance of some exemplary prediction models based on the one or more target metabolites may also be found in the following examples.

### EXAMPLES

### Material and Method

### 1. Study Cohorts and Sample Collection

In this study, two independent cohorts were enrolled. The discovery cohort is composed of 173 individuals, including 66 CD, 33 UC and 74 normal individuals. Matched serum and fecal samples of individuals in this cohort have been collected and further stored at −80° C. until been examined.

TABLE 26
Grouping standards and cohort composition
of the discovery cohorts.

| Group | Category | Number | Age (mean ± SD) | Total |
|---|---|---|---|---|
| Non IBD | Normal | 74 | 36.9 ± 10.53 | 74 |
| IBD | UC | 33 | 42 ± 13.11 | 99 |
| | CD | 66 | 29 ± 11.38 | |

In addition, serum samples of an independent modeling cohort were also been collected, including 54 UC, 37 CD, 35 normal and 74 colorectal adenoma and non-adenoma polyp individuals. These individuals were randomly divided into training set and testing set with a 6:4 ratio.

TABLE 27
Grouping standards and cohort composition
of the modeling cohorts.

| | | | Age | |
|---|---|---|---|---|
| Group | Category | Number | (mean ± SD) | Total |
| Non | Normal | 35 | 45.2 ± 4.05 | 109 |
| IBD | Non-adenoma | 26 | 44 ± 3.78 | |
| | polyps | | | |
| | Colorectal | 48 | 50.1 ± 3.72 | |
| | adenoma | | | |
| IBD | UC | 54 | 44.3 ± 10.52 | 91 |
| | CD | 37 | 46.4 ± 6.75 | |

### 2. Reagents and Equipment

### Equipment

Vortex mixer (Kylin-Bell Vortex X5)
20 μL, 100 μL, 200 μL, 1000 μL Pipettes and tips (Gilson)
High-speed microcentrifuge (Centrifuge 5415R)
Electronic balance (MettlerToledo AB104)
Centrifugal vacuum evaporator (TOMY CC-105)
Exion −20adxr Ultra Performance Liquid Chromatography system (shimadzu) coupled with a Triple Quad™ 4500MD LC-MS/MS system (AB Sciex)
ACQUITY UPLC BEH C18 Column (Shim-pack Velox C18 2.7 μm 2.1×100 mm)
R statistical scripting language (version 4.2.1)
AB Sciex Analyst software system (version 1.6.3)
Q Exactive Plus mass spectrometer fitted with UltiMate3000 LC series (ThermoFisher)
CORTECS (Waters) 1.6 μm C18+2.1*100 mm column

### Reagents and Supplies

LC-MS-grade methanol (Thermo Fisher Scientific)
LC-MS-grade acetonitrile (Thermo Fisher Scientific)
LC-MS-grade formic acid (Thermo Fisher Scientific)
Ammonium acetate, LC-MS grade (Thermo Fisher Scientific)
13C cholic acid (Sigma-Aldrich)
Ultrapure water, HPLC grade (watsons)
Centrifuge tubes (1.5 ml; Axygen, cat. no. MCT-150-C)
10 μL, 200 μL, 1000 μL Pipette tips (Axygen)

### Solutions

¹³C labeled cholic acid stock solution (internal standard): weigh 10.8 mg cholic acid and dissolve into 1080 μL methanol, violently vortex until total dissolution. The final concentration of the stock solution is 10 mg/ml.

Precipitation solution: add 120 ul 13C cholic acid stock solution into 300 ml methanol and mix.

### 3 Metagenome Sequencing

Fecal samples of the individuals involved in the discovery cohort were used for DNA extraction by QIAamp DNA Stool Mini Kit (QIAGEN), among which 151 DNA samples passed the quality control. Whole-genome shotgun libraries preparation and subsequent metagenomic sequencing were carried out on the HiSeq 4000 platform (Illumina) with 150 base pair, paired-end reads at Shanghai OE Biotech Co. Ltd, targeting >10 Gb of sequence per sample.

Raw sequencing data was processed using Trimmomatic V0.36, including adapter trimming, depleting low quality reads or base pairs, as well as removing host contaminations by mapping against the human genome (hg19) with Bowtie 2. Afterwards, clean reads were constructed and further taxonomically profiled using MetaPhIAn2 version 2.2.0 with default parameters. In total, 8705 microbiome species were profiled and among them, species with relative abundances more than 0.01% in at least 3 individual were selected and considered for further model construction and microbiome-metabolome co-relation analysis.

### 4 Untargeted Metabolomics Detection

### I. Metabolites Extraction

All samples were prepared by the salting-out process for extraction. To 60 μL of serum, 6 μL Internal Standard solution (L-Tyrosine-(phenyl-3,5-d2) 100 μg/ml, Sigma-Aldrich; 13C-Cholic Acid 10 ug/ml, Cambridge Isotope Laboratories; Doxercalciferol 60 ug/ml, MedChem Express), 240 μL ACN:IPA (3:1, both ThermoFisher), 60 μL ammonium formate (0.5 g/ml) were added and vigorously mixed. After centrifugation at 18,000 g for 5 min, 200 μL supernatant was dried by Centrivap cold-trap centrifugation (Labconco), resuspended in 75 μL 55% methanol (ThermoFisher) containing 0.1% FA (ThermoFisher), and centrifuged at 13,000 rpm for 3 min at RT. Supernatant was used for metabolic analysis using a Q Exactive Plus mass spectrometer fitted with UltiMate3000 LC series (ThermoFisher) at a positive and negative of HESI (both 130-1200 m/z), respectively. A CORTECS (Waters) 1.6 μm C18+2.1*100 mm column, maintained at 35° C., was set to a 0.3 mL/min flow rate and 5 μL sample injection. Mobile phase A (ACN containing 0.1% FA) was applied as a gradient (from 5% to 45% at 0.5-14 min, 75% at 32 min, 80% at 42 min, 100% at 50-55 min and back to 5% for 5 min). Mobile phase B was Merck Millipore water containing 0.1% FA.

Metabolomics raw data were pre-processed and normalized by XC-MS. Metabolites with the maximum average abundance more than 5000 in either UC, CD, or normal group were collected into the omics profile. The spectra of significantly different permutations and all other metabolites separately were scanned in SIM and PRM modes and Full MS/dd-MS2 mode with reference with HMDB, mzCloud, and Chemspider databases in Compound Discover (v3.1).

### II. Quality Control

Equal volume (15 ul) of serum derived from each individual from the discovery cohort were pooled together, and the pooled sample was used as the QC sample. At least 15 QC samples were arranged in each detection batch. Peak areas of metabolites for all individuals were normalized to the same QC sample before subsequent analysis.

### 5 Gut Microbiome-Serum Metabolome Correlation Analysis

Pairwise correlation coefficients using Spearman's correlation coefficients between gut microbiome species and serum metabolites were carried out for the 66 CD patients in individuals of the matched cohort. Correlation coefficient and FDR for each species-metabolite pair was calculated and considered significantly associated with the cut off of FDR equal to or less than 10%.

### 6 Targeted Metabolomics Detection

### I. Metabolites Extraction

For metabolite extraction in targeted metabolomics detection, 10 μL internal standard solution (5 μg/mL 13C-Cholic Acid) was added to 80 μL serum with 150 μL acetonitrile:isopropanol (4:1 by volume, Thermo Fisher), 50 μL ammonium formate (0.5 g/mL), vortexing and followed by centrifugation at 17,949 g for 5 min. Then, 60 μL supernatant was diluted within 150 μL HPLC-grade water before use.

### II. Detection Method

The pseudo-targeted method was developed in dependent of pure standards, similar to what has been described by Fujian Zheng et. al (Nature Protocols, 2020), determining relative level of all metabolites in the identified panel by using the same reference pool sample for normalizing abundances for each individual. Targeted metabolomics detection was carried on AB SCIEX Triple Quad™ 4500 system and run-in separate ion modes (positive and negative). The mobile phase and the column used for reversed-phase liquid chromatography were used as listed in the table below. The injection volume was 15 μL for each mode. Metabolites were eluted from the column at a flow rate of 0.3 ml/min with a gradually increasing concentration of mobile phase B, 12% of mobile phase B initially, to 60% of the mobile phase B after 2.5 min. A linear 60%-85% and 85%-100% phase B gradient was set at 6 min and 8.5 min. Delustering potentials and collision energies were optimized from the quality control samples of the control group. Metabolite peaks were integrated using the Sciex Analyst 1.6.3 software.

TABLE 28
Details of chromatography parameters.
Positive mode

| Column | Shimadzu shim-pack Velox | | | |
|---|---|---|---|---|
| | C18(50*2.1 mm, 2.7 μm) | | | |
| Temperature | 45 C. ° | | | |
| Autosampler temperature | 15 C. ° | | | |
| Mobile phase | A: 0.1% formic acid-water | | | |
| | B: 0.1% formic acid-acetonitrile | | | |
| | | | mobile | mobile |
| | | flow | phase | phase |
| | time(min) | rate(ml/min) | A (%) | B (%) |
| Mobile phase gradient | 0.50 | 0.4 | 70 | 30 |
| | 2.50 | 0.4 | 50 | 50 |
| | 3.70 | 0.4 | 20 | 80 |
| | 3.71 | 0.4 | 2 | 98 |
| | 4.99 | 0.4 | 2 | 98 |
| | 5.00 | 0.4 | 80 | 20 |
| | 6.00 | 0.4 | 80 | 20 |

| | |
|---|---|
| Autosampler wash | 80% formic acid |
| Sample size | 15 μL |
| Pre-balance before sample | inject wash solution once |
| injection | |

Negative mode

| Column | Shimadzu shim-pack Velox | | | |
|---|---|---|---|---|
| | C18(50*2.1 mm, 2.7 μm) | | | |
| Temperature | 45 C. ° | | | |
| Autosampler temperature | 15 C. ° | | | |
| Mobile phase | A: 0.1% formic acid-water | | | |
| | B: 0.1% formic acid-acetonitrile | | | |
| | | | mobile | mobile |
| | | flow | phase | phase |
| | time(min) | rate(ml/min) | A (%) | B (%) |
| Mobile phase gradient | 0.10 | 0.4 | 75 | 25 |
| | 1.50 | 0.4 | 65 | 35 |
| | 2.80 | 0.4 | 65 | 35 |
| | 2.81 | 0.4 | 60 | 40 |
| | 4.00 | 0.4 | 60 | 40 |
| | 6.00 | 0.4 | 40 | 60 |
| | 7.00 | 0.4 | 20 | 80 |

| | |
|---|---|
| Autosampler wash | 80% formic acid |
| Sample size | 15 μL |
| Pre-balance before sample | inject wash solution once |
| injection | |

### IV. Parameters for Mass Spectrum

TABLE 29
details of ion source parameters under positive and negative modes

| | | | | | |
|---|---|---|---|---|---|
| | | Ion | | | |
| Curtain | Collision | Spray | | Ion | Ion |
| Gas | Gas | Voltage | Temperature | Source | Source |
| (CUR) | (CAD) | (IS) | (TEM) | Gas1(Gas1) | Gas2(Gas2) |

Turbo Ion spray (ESI+)

| | | | | | |
|---|---|---|---|---|---|
| 30 | 8 | 5200 | 500 | 45 | 45 |

Turbo Ion spray (ESI−)

| | | | | | |
|---|---|---|---|---|---|
| 30 | 8 | −4500 | 500 | 50 | 50 |

TABLE 30
Scheduled MRM under positive and negative modes.
Turbo Ion spray (ESI+)

| | | | |
|---|---|---|---|
| MRM detection | 60 sec | Target Scan Time | 0.4 sec |

Turbo Ion spray (ESI−)

| | | | |
|---|---|---|---|
| MRM detection | 60 sec | Target Scan Time | 0.4 sec |

### V. Quality Control

### QC Sample

Equal volume (15 ul) of serum derived from each individual from this cohort were pooled together, and the pooled sample was used as the QC sample. At least 6 QC samples were arranged in each detection batch. Peak areas of metabolites for all individuals were normalized to the same QC sample before subsequent analysis.

### 7 Data Analysis

Data preprocessing, statistical analysis, and predictive model building were conducted using R programming (v4.2.1). Relative abundances for each metabolites were used in this study. Raw abundances of metabolites for all individuals were normalized by Loess, and their ratios to the abundances of the same QC sample were calculated (the relative abundance) and used for subsequent analysis.

### 8 Selection of the Metabolites for the IBD Diagnosis and UC/CD Specifying Models

To select the metabolite features for the diagnosis models, the LASSO algorithm was implemented with 10-fold cross validation for feature selection from the serum metabolomics data. The selected feature was subsequently used to construct prediction model by logic regression in the training cohort, and the cut off value was set at the point to achieve the highest accuracy.

### Example 1 Untargeted Metabolomics Profiling in Serum from the Discovery Cohort Revealed Significant Reprogramming Between Normal and IBD Patients

Previous studies have revealed a significant shift of gut microbiome structure in IBD patients, and metabolic activity were also changed, including a reduced production of secondary bile acid, while elevation of sphingolipids, and carboxamide acid pathways. Additionally, gut epithelium permeability was also significantly increased in IBD patients, and UC patients were also higher than CD group. These may cause significant changes of gut content into the circulating system, and thus may contribute to the significant reprograming of serum metabolites, which could provide potential approach for biomarker discovery of IBD diagnosis and UC/CD subtyping.

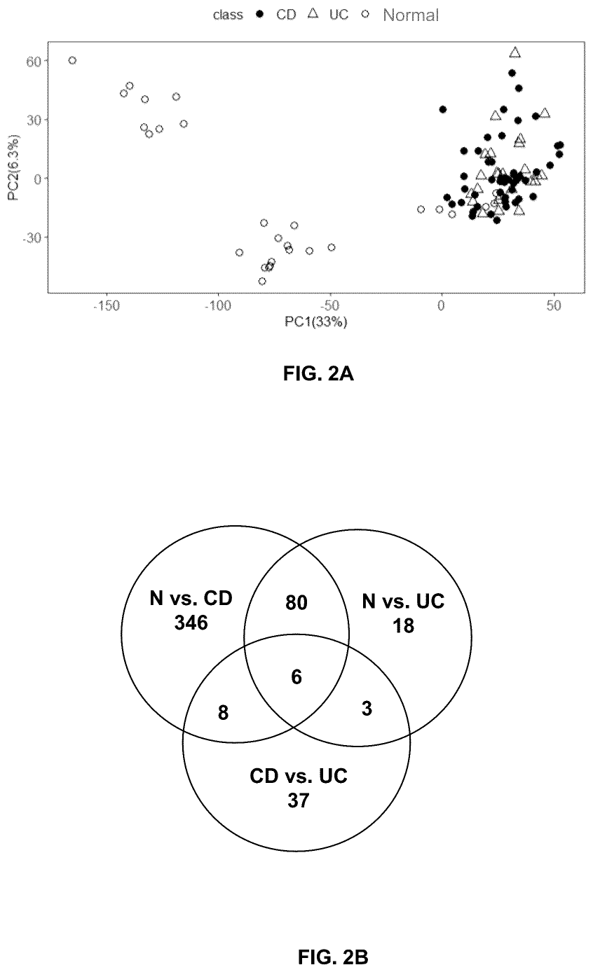

To investigate the changes of the serum metabolome between normal and IBD patients, untargeted metabolome profiling was carried out by UPLC-MS within a cross-sectional discovery cohort. Within this cohort, untargeted metabolome of 50 CD, 26 UC and 28 normal individuals passed quality control and were used for subsequent analysis. Equal volume of serum samples were pooled together as QC sample to normalize accuracy and repeatability within each batches. After filtering out low-abundance signals (mean abundance<50000 in all groups) and non-accurate signals (CV % in QC samples >30%), all metabolites that showed significantly different abundances between either pair of groups (p value<0.005, fold change >1.2 or <0.8) were explored. Distributions of all samples in a principal component analysis (PCA) plot was displayed according to these metabolites (FIG. 2A), observing that the UC and CD individuals were similar, while normal group could be clearly distinguished from these two groups. On further metabolite annotation and comparison of the three lists of significantly altered metabolites, 461 altered metabolites in UC or CD individuals compared to normal individuals, as well as 54 altered metabolites between UC and CD individuals (FIG. 2B) were acquired.

### Example 2 Reprogramming of Microbiome Structure in IBD Patients

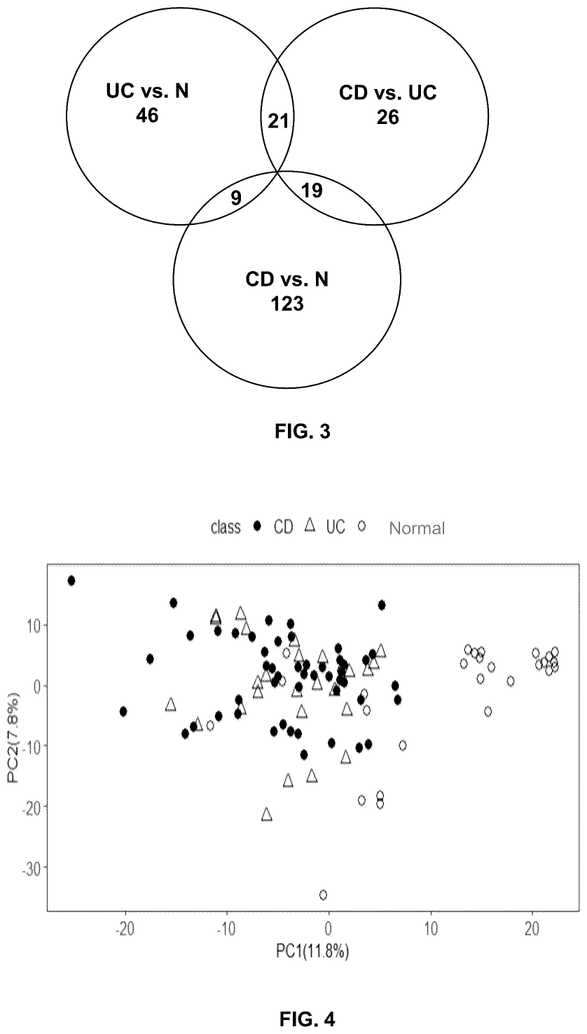

The reprogramming of serum metabolome may attributes to both gut microenvironment and host itself. To further evaluate the contribution of gut microbiome to these altered serum metabolites and reveal mechanistic links, metagenome sequencing was carried out using fecal samples within the discovery cohort. In total, metagenome data of 151 individuals, including 65 CD, 12 UC, and 74 normal individuals passed quality control and been used for subsequent analysis. Taxonomic profiling of the metagenome data revealed 8706 microbiome species, and significant shifts could be observed in gut microbiome between IBD and non-IBD individuals, while comparably less significant between UC and CD patients, and significantly altered gut microbiome species have also been displayed in the venn diagram (FIG. 3). As used herein, a non-IBD individual or a non-IBD subject refers to a normal subject or a subject having colorectal polyp. Specific microbiome species that have been reported to be pathogenic or protective, including Faecalibacterium prausnitzii, R. gnavus, exhibit consistent trend with previous findings, further supporting the quality of our metagenome sequencing data.

To profile microbiome associated serum metabolites, Spearman's correlation coefficient analysis was carried out, using the 1286 species with relative abundance higher than 0.01% in at least 3 individual and annotated metabolites that were significantly different between IBD and non-IBD individuals, or between UC and CD patients, setting the cut-off at FDR≤10.0%. In total, co-related species-metabolite pairs were found, with 246 IBD vs non-IBD significantly different metabolites could be matched to the 728 IBD related gut microbiomes, while 96 UC vs CD significantly different metabolites could be matched to the 462 gut microbiomes. Based on all these gut microbiome co-related serum metabolites, a clear separation between normal and IBD patients could also be observed (FIG. 4), indicating the contribution of gut microbiome on serum metabolome reprogramming in IBD patients.

### Example 3 Establishing Diagnostic Model for IBD Diagnosis and UC/CD Specification in the Training Cohort

I. Predictive Accuracy of these Metabolites Panel in the Discovery Cohort.

Based on these metabolites described in above table 31, a LASSO algorithm was performed with 10-fold cross validation for feature selection from the targeted serum metabolomics data of the discovery cohort to seek for key metabolite biomarkers for detecting IBD or specifying UC and CD. 156 metabolite features have been selected and used for subsequent model construction. The predictive accuracy of this metabolite panel in distinguishing UC/CD vs. non-IBD, as well as UC vs. CD patients in the discovery cohort was evaluated.

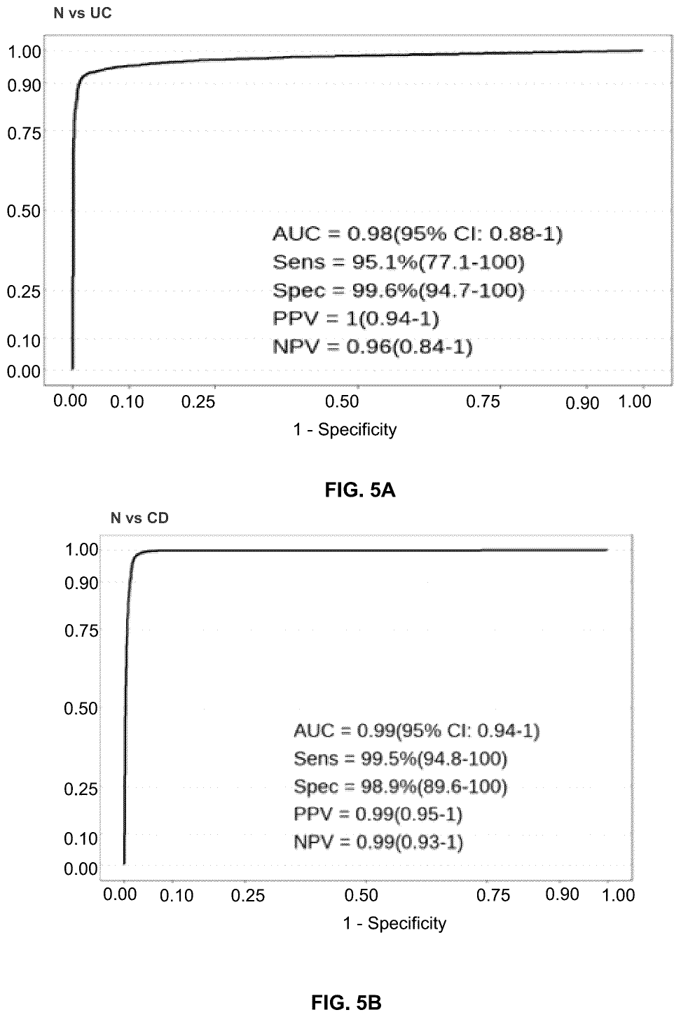

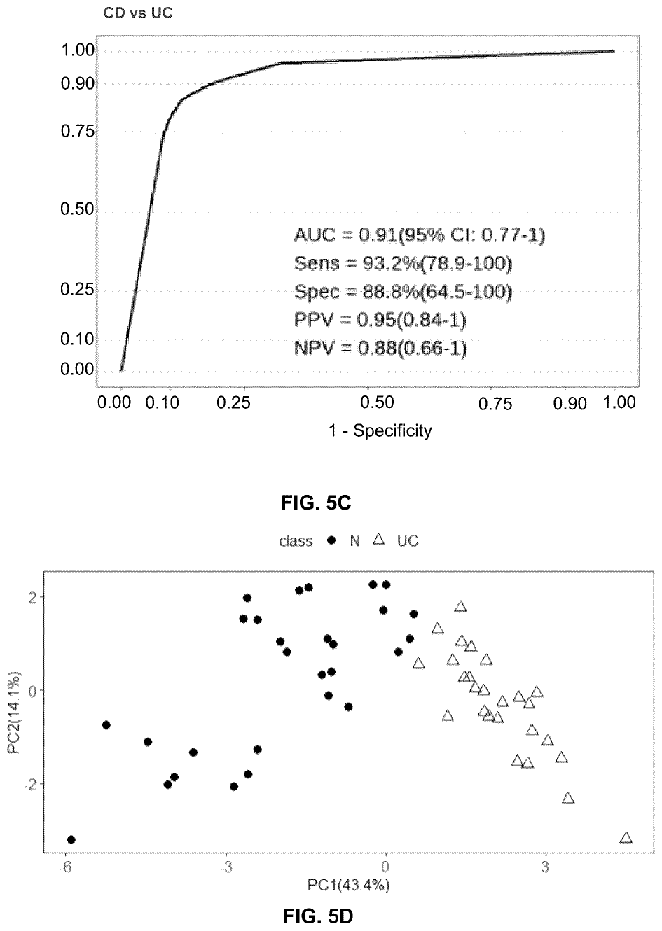

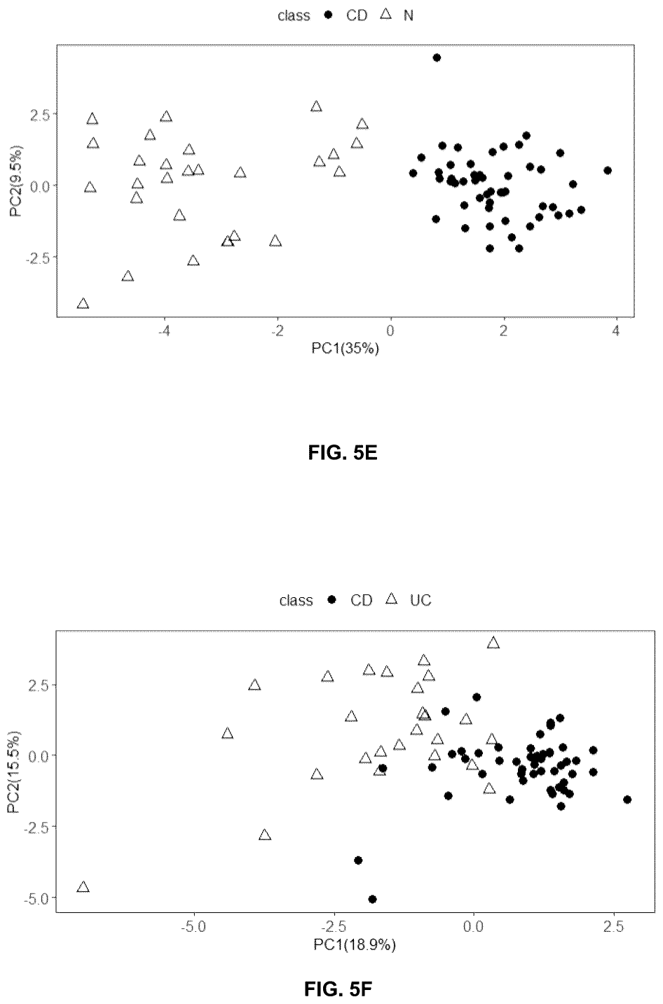

First, based on the relative abundances detected by untargeted metabolomic profiling of these 156 metabolites, the normal individuals and UC or CD patients in the discovery cohort could all be accurately distinguished, reaching an area under the curve (AUC) of 0.98 (95% CI 0.88 to 1.00) and 0.99 (95% CI 0.94 to 1.00), respectively (FIG. 5A, B). Additionally, based on this panel of serum metabolites, another model was used to specify UC and CD, yielding an AUC of 0.91 in the discovery cohort (FIG. 5C). PCA plots (FIG. 5D-5F) also showed clear separations between the normal individuals and IBD patients, as well as between UC and CD patients using respective models, further indicates the predictive value of these metabolites.

### II. Candidate Feature Selection Based on Untargeted Metabolome and Transition to MRM Based Targeted Detection.

Based on these annotated serum metabolites that both gut-microbiome associated and also showed significant alternation either between normal and IBD, or between UC and CD patients, characteristic precursor and daughter ion pairs of 156 metabolites could be acquired, and further carried out their transitions based on MRM detection with the 4500MD UPLC-MS system. This transition process yields a panel of 156 serum metabolites that showed potential for discriminating UC and CD from normal individuals. These metabolites were then selected to constitute the IBD diagnostic and UC/CD subtyping panel.

As is shown in table 31 below, based on the parameters described above, 156 metabolites were involved in the diagnostic panel, and used for subsequent model construction.

TABLE 31
List of metabolites involved in the IBD
diagnostic and UC/CD subtyping panel.

| | | | Del- |
|---|---|---|---|
| Meta ID | MASS (+/−) | Compound | ta(ppm) |
| BN001 | 319.228(−) | 5_HETE | 0 |
| BN003 | 319.228(−) | 8_HETE | 0 |
| BN004 | 319.228(−) | 9_HETE | 0 |
| BN006 | 319.228(−) | 12_HETE | 0 |
| BN012 | 343.228(−) | 14(S)-HDHA | 0 |
| BN013 | 343.228(−) | 17(S)_HDHA | 0 |
| BN015 | 350.210(−) | Sphingosine-1-phosphate (d16:1) | 0 |
| BN016 | 313.239(−) | Octadecane dioic acid | 0 |
| BN017 | \ | Epitestosterone Sulfate | 0 |
| BN020 | 389.270(−) | 3α-Hydroxy-6-OXO-5α- | 0 |
| | | Cholan-24-OIC Acid | |
| BN021 | 405.265(−) | 3-Dehydrocholic Acid | 0 |
| BN022 | 405.265(−) | 5α-Cholanic Acid- | 0 |
| | | 3α, 7β-Diol-6-One | |
| BN023 | 301.218(−) | Eicosapentaenoic Acid | 0 |
| BN025 | 361.202(−) | Hydrocortisone | 0 |
| BN027 | 480.310(−) | 1-Stearoyl-2-Hydroxy-sn- | 0 |
| | | Glycero-3-Phosphoethanolamine | |
| BN028 | 159.067(−) | Pimelic acid | 0 |
| BN029 | 187.098(−) | Azelaic acid | 0 |
| BN030 | 303.233(−) | Arachidonic acid | 0 |
| BN035 | 329.234(−) | 9(S),10(S),13(S)_Trihydroxy_{—} | 0 |
| | | 11(E)_Octadecenoic Acid | |
| BP002 | 177.102(+) | S-(−)-Cotinine | 0 |
| BP003 | 379.284(+) | 2-Arachidonoyl Glycerol | 0 |
| BP006 | 372.311(+) | Myristoyl-L-carnitine | 0 |
| BP007 | 286.201(+) | trans-2-octenoyl-I-carnitine | 0 |
| BP009 | 355.284(+) | 1-Linoleoyl-rac-glycerol | 0 |
| BP010 | 398.326(+) | trans-2-Hexadecenoyl-L-carnitine | 0 |
| BP011 | 316.248(+) | Decanoyl-L-carnitine | 0 |
| BP012 | 261.193(+) | (+)-Hexanoyl carnitine chloride | 0 |
| BP013 | 303.232(+) | 17-Alpha-Methyltestosterone | 0 |
| C004 | 267.073 (−) | C10H12N4O5 | 2 |
| C006 | 295.229 (−) | C18H32O3 | 2 |
| C008 | 327.256 (−) | C19H36O4 | 5 |
| C009 | 355.158 (−) | C22H20N4O | 4 |
| C011 | 381.174 (−) | C16H30O10 | 7 |
| C012 | 398.132 (−) | C19H21N5O3S | 7 |
| C015 | 407.280 (−) | C24H40O5 | 0 |
| C016 | 427.163 (−) | C24H23F3N2O2 | 2 |
| C017 | 439.379 (−) | C27H52O4 | 0 |
| C019 | 447.312 (−) | C27H44O5 | 1 |
| C021 | 468.308 (−) | C29H43NO2S | 30 |
| C026 | 480.310 (−) | C25H43N3O6 | 4 |
| C027 | 481.354 (−) | C31H46O4 | 45 |
| C030 | 528.310 (−) | C30H47N3O5 | 65 |
| C031 | 540.331 (−) | C34H43N3O3 | 14 |
| C032 | 580.235 (−) | C33H35N5O5 | 37 |
| C033 | 581.241 (−) | C33H34N4O6 | 1 |
| C035 | 590.346 (−) | C33H45N5O5 | 19 |
| C036 | 642.396 (−) | C31H57N5O9 | 19 |
| C041 | 499.288 (−) | C26H44O9 | 7 |
| C043 | 511.302 (−) | C31H44O6 | 9 |
| C102 | 181.072 (+) | C7H8N4O2 | 1 |
| C110 | 286.201 (+) | C15H27NO4 | 1 |
| C112 | 315.134 (+) | C15H17F3N2O2 | 9 |
| C114 | 317.195 (+) | C19H37NO6 | 3 |
| C116 | 330.263 (+) | C18H35NO4 | 2 |
| C119 | 337.273 (+) | C21H36O3 | 2 |
| C120 | 355.283 (+) | C21H38O4 | 4 |
| C122 | 372.300 (+) | C24H37NO2 | 28 |
| C124 | 398.325 (+) | C23H43NO4 | 4 |
| C128 | 428.363 (+) | C28H43O3 | 79 |
| C129 | 442.352 (+) | C25H47NO5 | 2 |
| C131 | 468.308 (+) | C22H46NO7P | 1 |
| C132 | 480.134 (+) | C23H21N5O5S | 1 |
| C135 | 195.087 (+) | C8H10N4O2 | 2 |
| C136 | 287.204 (+) | C19H26O2 | 14 |
| C137 | 302.215 (+) | C19H27NO2 | 11 |
| C139 | 341.306 (+) | C21H40O3 | 2 |
| C144 | 357.280 (+) | C24H36O2 | 3 |
| C145 | 464.314 (+) | C23H46NO6P | 2 |
| C146 | 482.324 (+) | C23H48NO7P | 0 |
| C147 | 506.323 (+) | C25H47NO9 | 18 |
| C148 | 508.340 (+) | C25H50NO7P | 1 |
| C149 | 508.340 (+) | C28H45NO7 | 26 |
| C150 | 530.324 (+) | C27H48NO7P | 1 |
| DS01 | 407.281(−) | CA (Cholic Acid) | 0 |
| DS02 | 391.286(−) | CDCA (Chenodeoxycholic Acid) | 0 |
| DS03 | 391.286(−) | DCA (Deoxycholic Acid) | 0 |
| DS04 | 464.302(−) | GCA (Glycocholic Acid Hydrate) | 0 |
| DS05 | 448.307(−) | GCDCA (Glycochenodeoxycholic Acid) | 0 |
| DS07 | 432.312(−) | GLCA (Glycolithocholic Acid) | 0 |
| DS10 | 391.286(−) | UDCA (Ursodeoxycholic Acid) | 0 |
| DS11 | \ | 5β-CAA-3β, 12α-2K | 0 |
| X004 | 239.092 (−) | C12H16O5 | 2 |
| X006 | 263.104 (−) | C13H16N2O4 | 1 |
| X011 | 311.223 (−) | C18H32O4 | 1 |
| X013 | 313.238 (−) | C18H34O4 | 1 |
| X016 | 319.228 (−) | C20H32O3 | 0 |
| X023 | 367.158 (−) | C19H28O5S | 1 |
| X024 | 369.174 (−) | C19H30O5S | 0 |
| X032 | 403.158 (−) | C25H24O5 | 7 |
| X036 | 425.201 (−) | C23H27FN4O3 | 4 |
| X055 | 526.315 (−) | C27H46NO7P | 40 |
| X066 | 592.362 (−) | C29H56NO9P | 0 |
| X070 | 646.427 (−) | C34H66NO8P | 28 |
| X082 | 353.212 (−) | C19H26N6O | 7 |
| X154 | 302.196 (+) | C16H23N5O | 5 |
| X160 | 316.247 (+) | C17H33NO4 | 4 |
| X166 | 352.224 (+) | C16H34NO5P | 2 |
| X183 | 490.300 (+) | C27H41F2N5O | 72 |
| X188 | 542.324 (+) | C28H48NO7P | 0 |
| X278 | 289.106 (−) | C11H18N2O7 | 6 |
| X280 | 447.312 (−) | C30H40O3 | 48 |
| X281 | 447.312 (−) | C27H44O5 | 1 |
| X285 | 512.336 (−) | C26H43NO7S | 131 |
| X286 | 512.336 (−) | C29H41F2N5O | 30 |
| X289 | 536.299 (−) | C29H47NO8 | 45 |
| X292 | 187.007 (−) | C7H8O4S | 0 |
| X293 | 204.067 (−) | C11H11NO3 | 2 |
| X401 | 222.114 (−) | C12H17NO3 | 2 |
| X403 | 239.092 (−) | C13H12N4O | 8 |
| X407 | 314.103 (−) | C17H17NO5 | 1 |
| X408 | 317.212 (−) | C20H30O3 | 1 |
| X409 | 335.259 (−) | C20H32O4 | 108 |
| X411 | 345.243 (−) | C22H34O3 | 2 |
| X412 | 353.164 (−) | C19H22N4O3 | 6 |
| X508 | 212.020 (+) | C7H2F5NO | 33 |
| X513 | 305.247 (+) | C20H32O2 | 2 |
| X515 | 335.151 (+) | C19H18N4O2 | 2 |
| X519 | 364.084 (+) | C11H18N5O7P | 49 |
| X525 | 563.427 (+) | C34H58O6 | 6 |
| X650 | 150.056(−) | C8H9NO2 | 5 |
| X651 | 188.987(−) | C6H6O5S | 1 |
| X652 | 194.046(−) | C9H9NO4 | 1 |
| X653 | 194.046(−) | C9H9NO4 | 1 |
| X654 | 201.114(−) | C10H18O4 | 6 |
| X655 | 201.114(−) | C10H18O4 | 3 |
| X656 | 212.003(−) | C4H8NO7P | 28 |
| X657 | 212.003(−) | C8H7NO4S | 1 |
| X658 | 271.05(−) | C15H12O5 | 26 |
| X659 | 295.228(−) | C18H32O3 | 1 |
| X660 | 301.202(−) | C17H26N4O | 3 |
| X661 | 327.099(−) | C14H17NO8 | 10 |
| X662 | 355.228(−) | C21H31F3O | 7 |
| X663 | 385.169(−) | C22H26O6 | 9 |
| X664 | 413.201(−) | C23H30N2O5 | 18 |
| X665 | 419.316(−) | C26H44O4 | 1 |
| X666 | 445.19(−) | C24H30O8 | 8 |
| X667 | 453.321(−) | C26H46O6 | 2 |
| X668 | 463.342(−) | C28H48O5 | 1 |
| X669 | 465.246(−) | C25H38O8 | 6 |
| X670 | 517.39(−) | C32H54O5 | 1 |
| X671 | 130.065(+) | C5H3D3N2O2 | 26 |
| X672 | 130.065(+) | C9H7N | 1 |
| X673 | 169.086(+) | C9H12O3 | 1 |
| X674 | 195.113(+) | C10H14N2O2 | 1 |
| X675 | 212.02(+) | C6H14NO2Se | 6 |
| X676 | 247.144(+) | C9H18N4O4 | 15 |
| X677 | 251.127(+) | C14H18O4 | 2 |
| X678 | 285.206(+) | C16H28O4 | 2 |
| X679 | 288.289(+) | C17H37NO2 | 2 |
| X680 | 303.231(+) | C20H30O2 | 2 |
| X681 | 337.273(+) | C21H36O3 | 2 |
| X682 | 368.087(+) | C16H15F2N3O3S | 3 |
| X683 | 397.183(+) | C22H24N2O5 | 17 |
| X684 | 399.237(+) | C21H34O7 | 2 |
| X685 | 411.198(+) | C20H30N2O5S | 8 |
| X686 | 510.355(+) | C25H52NO7P | 1 |

### Example 4 Targeted Model Establishment and Examination in the Modeling Cohort: N Vs UC, N Vs CD, UC Vs CD; IBD Vs Adenomas and Non-Adenoma Polyps

Based on the metabolites panel by targeted detection described in table 31, an independent modeling cohort was enrolled, including 54 UC, 37 CD, 35 normal individuals, as well as 74 colorectal adenoma and non-adenoma polyps patients, and performed targeted metabolomics detection in these individuals. These individuals were randomly divided into training set and testing set with a 6:4 ratio. The composition of the training and the testing set were listed in table 32. IBD diagnosis and UC/CD specification models were subsequently developed in the training set and examined in the testing set, based on targeted detection of these metabolites enrolled in this panel.

TABLE 32
Patient composition in the training set of the modeling cohort.

| | | | Age | |
|---|---|---|---|---|
| Group | Category | Number | (mean ± SD) | Total |
| Non | Normal | 21 | 45.6 ± 4.81 | 109 |
| IBD | Non-adenoma | 16 | 44.4 ± 3.65 | |
| | polyps | | | |
| | Colorectal | 29 | 49.4 ± 3.93 | |
| | adenoma | | | |
| IBD | UC | 33 | 46.5 ± 14.39 | 91 |
| | CD | 23 | 46.7 ± 11 | |

TABLE 33
Patient composition in the testing set of the modeling cohort.

| | | | Age | |
|---|---|---|---|---|
| Group | Category | Number | (mean ± SD) | Total |
| Non | Normal | 14 | 45.3 ± 3.43 | 109 |
| IBD | Non-adenoma | 10 | 43.3 ± 4.08 | |
| | polyps | | | |
| | Colorectal | 19 | 51.3 ± 3.12 | |
| | adenoma | | | |
| IBD | UC | 21 | 44.6 ± 11.43 | 91 |
| | CD | 14 | 40.4 ± 8.7 | |

### I. Diagnostic Model to Discriminate Normal Individuals and UC Patients.

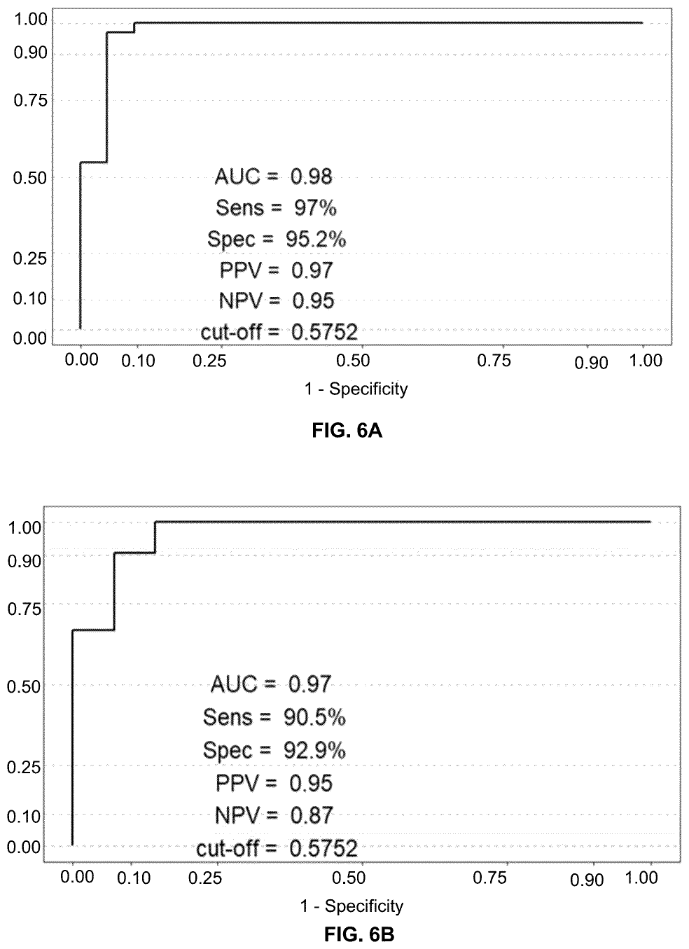

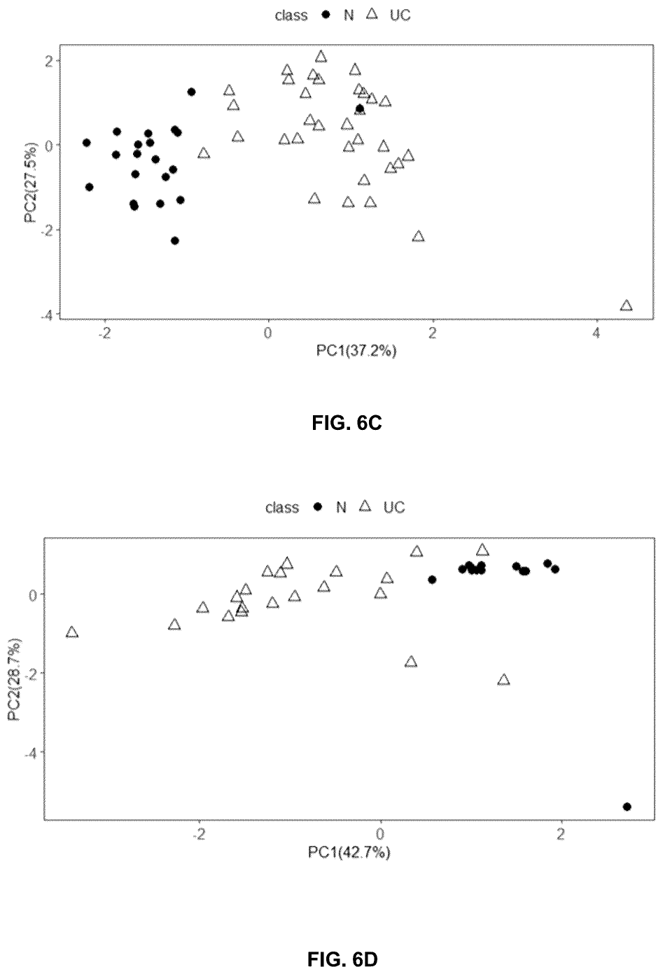

Based on the metabolites panel by targeted detection described in table 31, fold change and p value between the 21 normal and 33 UC individuals within the training set was calculated (described in table 32). Significantly altered serum metabolites (fold change >1.2 or <0.8, p value<0.05) were filtered out and subsequent feature selection for model construction were carried out using the LASSO algorithm. Subsequently, prediction models were constructed based on logistic regression to discriminate UC and normal individuals in the training set, achieving an AUC of 0.98 (sensitivity=97%, specificity=95.2%, PPV=0.97, NPV=0.95) (FIG. 6A). To further evaluate performance of this model, the performance of the N vs UC diagnostic model in the testing set was evaluated. This model could also yields an AUC of 0.97 (sensitivity=90.5%, specificity=92.9%, PPV=0.95, NPV=0.87) to discriminate UC patients from normal individuals in the testing set (FIG. 6B), and PCA plots (FIG. 6C-6D) also showed clear separations between the normal individuals and IBD patients in both training and the testing set. Significantly altered serum metabolites were listed in Table 34, Metabolites used for the N vs UC diagnostic model were listed in Table 35. In addition, using either 1 or combination of 2 or 3 metabolites listed in Table 36 could also acquire promising performances for N vs UC diagnosis. Metabolites or metabolites combinations and their respective performances were listed in table 37.

TABLE 34
List of significantly altered serum metabolites in the N vs UC

| | average | average | | |
|---|---|---|---|---|
| | abundance | abundance | | |
| | in N | in UC | | |
| Meta ID | samples | samples | Foldchange | pvalue |
| BN001 | 8183 | 4595 | 0.562 | 0.000317 |
| BN012 | 33300 | 19727 | 0.592 | 0.041062 |
| BN015 | 74240 | 40756 | 0.549 | 4.08E−07 |
| BN021 | 970 | 3598 | 3.71 | 0.04481 |
| BN022 | 6033 | 22255 | 3.69 | 0.036227 |
| BN023 | 301821 | 162918 | 0.54 | 0.033105 |
| BN025 | 26725 | 43622 | 1.63 | 0.027917 |
| BN028 | 42492 | 12470 | 0.293 | 0.001876 |
| BN029 | 15303992 | 3082529 | 0.201 | 5.07E−09 |
| BP007 | 86670 | 52196 | 0.602 | 0.002942 |
| BP010 | 100984 | 136450 | 1.35 | 0.004077 |
| BP011 | 1802788 | 2714688 | 1.51 | 0.007198 |
| BP012 | 215618 | 285704 | 1.33 | 0.00457 |
| BP013 | 17819 | 6402 | 0.359 | 7.7E−06 |
| C004 | 12081 | 130053 | 10.8 | 0.000512 |
| C009 | 655529 | 818418 | 1.25 | 0.003553 |
| C011 | 1738925 | 2250936 | 1.29 | 0.001598 |
| C012 | 2938 | 4919 | 1.67 | 0.005708 |
| C017 | 872740 | 490361 | 0.562 | 2.38E−05 |
| C019 | 35887 | 15295 | 0.426 | 9.89E−05 |
| C021 | 453563 | 352909 | 0.778 | 0.019039 |
| C026 | 16201509 | 12183534 | 0.752 | 0.048569 |
| C027 | 127794 | 41602 | 0.326 | 2.30E−07 |
| C031 | 102535209 | 80079998 | 0.781 | 0.002869 |
| C035 | 4517101 | 3292966 | 0.729 | 0.049157 |
| C036 | 20228 | 32759 | 1.62 | 0.004528 |
| C043 | 75310 | 200107 | 2.66 | 0.002647 |
| C102 | 330441 | 23181 | 0.0702 | 0.0306 |
| C110 | 90706 | 50052 | 0.552 | 0.002439 |
| C114 | 12335 | 19002 | 1.54 | 0.009387 |
| C122 | 7544 | 14969 | 1.98 | 0.010973 |
| C124 | 238338 | 327004 | 1.37 | 0.003822 |
| C129 | 24784 | 34072 | 1.37 | 0.010495 |
| C131 | 6523205 | 3468244 | 0.532 | 0.003232 |
| C132 | 282499 | 170549 | 0.604 | 0.000153 |
| C135 | 490785 | 80946 | 0.165 | 0.04561 |
| C136 | 57750 | 31670 | 0.548 | 0.002995 |
| C145 | 326344 | 179917 | 0.551 | 0.000193 |
| C146 | 4313170 | 2147826 | 0.498 | 2.75E−05 |
| C147 | 95155 | 54743 | 0.575 | 0.000211 |
| C148 | 1652992 | 901971 | 0.546 | 0.001302 |
| C149 | 536898 | 355268 | 0.662 | 0.003119 |
| C150 | 46113 | 56804 | 1.23 | 0.002408 |
| DS02 | 487702 | 4289042 | 8.79 | 0.001911 |
| DS03 | 1929113 | 484792 | 0.251 | 0.009186 |
| DS04 | 109028 | 394572 | 3.62 | 0.000859 |
| DS05 | 1206246 | 3251447 | 2.7 | 0.003603 |
| DS07 | 25204 | 4574 | 0.181 | 0.01085 |
| DS10 | 267758 | 1811925 | 6.77 | 0.047948 |
| X004 | 4613023 | 1727995 | 0.375 | 3.77E−05 |
| X006 | 208696 | 374611 | 1.8 | 0.032418 |
| X013 | 348340 | 151753 | 0.436 | 0.047162 |
| X023 | 3934149 | 3084372 | 0.784 | 0.018454 |
| X055 | 7245417 | 3708044 | 0.512 | 4.00E−08 |
| X066 | 6309371 | 4110158 | 0.651 | 0.000383 |
| X070 | 860375 | 1329695 | 1.55 | 0.005485 |
| X082 | 415 | 1223729 | 2950 | 0.004411 |
| X160 | 1852331 | 2776187 | 1.5 | 0.008055 |
| X166 | 131134 | 72827 | 0.555 | 0.000354 |
| X183 | 149915 | 81008 | 0.54 | 0.001873 |
| X278 | 600761 | 323720 | 0.539 | 4.37E−05 |
| X281 | 38561 | 16977 | 0.44 | 0.000288 |
| X285 | 14329935 | 8147062 | 0.569 | 4.39E−05 |
| X286 | 1441386 | 826183 | 0.573 | 0.000202 |
| X289 | 240927 | 147317 | 0.611 | 0.000452 |
| X401 | 16246 | 9106 | 0.56 | 0.000364 |
| X403 | 11968117 | 4831677 | 0.404 | 9.56E−06 |
| X408 | 13573 | 7737 | 0.57 | 0.041423 |
| X409 | 3019 | 1942 | 0.643 | 0.013608 |
| X411 | 34827 | 21747 | 0.624 | 0.038337 |
| X412 | 41143 | 51096 | 1.24 | 0.003571 |
| X508 | 14027 | 214607 | 15.3 | 0.00013 |
| X515 | 9189 | 11758 | 1.28 | 0.017247 |
| X655 | 119679 | 531374 | 4.44 | 1.65E−05 |
| X667 | 13958334 | 890542 | 0.0638 | 0.0001487 |
| X650 | 578193 | 919326 | 1.59 | 0.007436 |
| X653 | 18666 | 81757 | 4.38 | 7.24E−06 |
| X683 | 453161 | 169935 | 0.375 | 0.008423 |
| X652 | 7764528 | 14364377 | 1.85 | 6.95E−06 |
| X680 | 183051 | 362440 | 1.98 | 0.009412 |
| X651 | 33837799 | 5718588 | 0.169 | 0.03781 |
| X654 | 1207668 | 531374 | 0.44 | 0.0138 |
| X656 | 4586171 | 2687496 | 0.586 | 0.04164 |

TABLE 35
List of metabolites involved in the N vs UC diagnostic model.

| NO | Meta ID |
|---|---|
| 1 | BN001 |
| 2 | BN029 |
| 3 | BP012 |
| 4 | C004 |
| 5 | C009 |
| 6 | C019 |
| 7 | C027 |
| 8 | C036 |
| 9 | C146 |
| 10 | C147 |
| 11 | C150 |
| 12 | DS02 |
| 13 | DS03 |
| 14 | DS04 |
| 15 | X004 |
| 16 | X082 |
| 17 | X403 |
| 18 | X508 |
| 19 | X655 |
| 20 | X667 |
| 21 | X650 |
| 22 | X653 |
| 23 | X683 |
| 24 | X652 |
| 25 | X680 |
| 26 | X651 |
| 27 | X654 |
| 28 | X656 |

TABLE 36
List of Metabolites or metabolites combinations and their
respective performances in the N vs UC diagnostic model.

| Meta ID | AUC | Sens | Spec | Meta ID | AUC | Sens | Spec |
|---|---|---|---|---|---|---|---|
| BN029 | 0.95 | 0.91 | 0.94 | BN029, X004 | 0.94 | 0.90 | 0.93 |
| C004 | 0.86 | 0.79 | 0.91 | C019, BP012 | 0.84 | 0.82 | 0.87 |
| C019 | 0.84 | 0.84 | 0.83 | C150, C146 | 0.85 | 0.87 | 0.79 |
| C027 | 0.88 | 0.89 | 0.82 | BN001, BN029, X082 | 0.95 | 0.91 | 0.96 |
| C146 | 0.81 | 0.81 | 0.80 | BN029, C027, X082 | 0.97 | 0.95 | 0.94 |
| DS04 | 0.80 | 0.86 | 0.72 | BP012, C009, C147 | 0.84 | 0.84 | 0.8 |
| X004 | 0.82 | 0.81 | 0.86 | C146, C150, X508 | 0.91 | 0.9 | 0.89 |
| X403 | 0.82 | 0.81 | 0.85 | C009, C147, X004 | 0.88 | 0.84 | 0.87 |
| X508 | 0.86 | 0.77 | 0.97 | BN001, C150, X004 | 0.82 | 0.82 | 0.83 |
| BN029, X082 | 0.96 | 0.92 | 0.96 | BN001, C036, X508 | 0.93 | 0.92 | 0.89 |
| C146, X508 | 0.91 | 0.91 | 0.89 | BN001, C147, X082 | 0.84 | 0.83 | 0.8 |
| C009, X004 | 0.85 | 0.83 | 0.84 | C019, C036, C150 | 0.83 | 0.82 | 0.84 |
| BN001, X004 | 0.82 | 0.82 | 0.85 | BN001, C004, C027 | 0.95 | 0.91 | 0.94 |
| X508, C036 | 0.88 | 0.84 | 0.91 | X004, C019, X403 | 0.83 | 0.82 | 0.85 |
| C147, X082 | 0.83 | 0.82 | 0.80 | C009, C027, DS02 | 0.9 | 0.92 | 0.87 |
| C147, BP012 | 0.84 | 0.83 | 0.82 | BP012, C027, C150 | 0.89 | 0.91 | 0.84 |
| C019, C150 | 0.84 | 0.86 | 0.82 | BN029, C146, C150 | 0.95 | 0.91 | 0.94 |
| C004, C027 | 0.94 | 0.90 | 0.95 | C147, DS03, DS04 | 0.85 | 0.8 | 0.89 |
| X004, C019 | 0.84 | 0.82 | 0.87 | BN029, C027, C147 | 0.95 | 0.93 | 0.92 |
| C009, DS02 | 0.83 | 0.86 | 0.78 | BN029, DS02, X004 | 0.96 | 0.97 | 0.94 |
| BN029, C150 | 0.94 | 0.91 | 0.94 | BP012, C019, DS02 | 0.91 | 0.88 | 0.93 |
| C147, C027 | 0.89 | 0.89 | 0.85 | C146, C147, C150 | 0.85 | 0.86 | 0.79 |

### II. Diagnostic Model to Discriminate Normal Individuals and CD Patients.

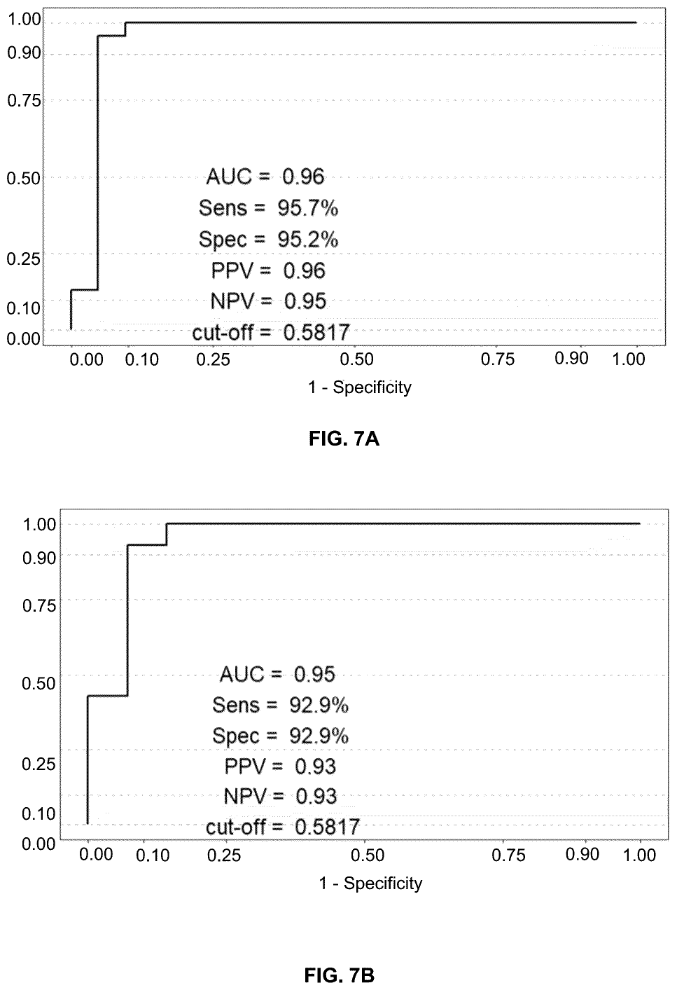

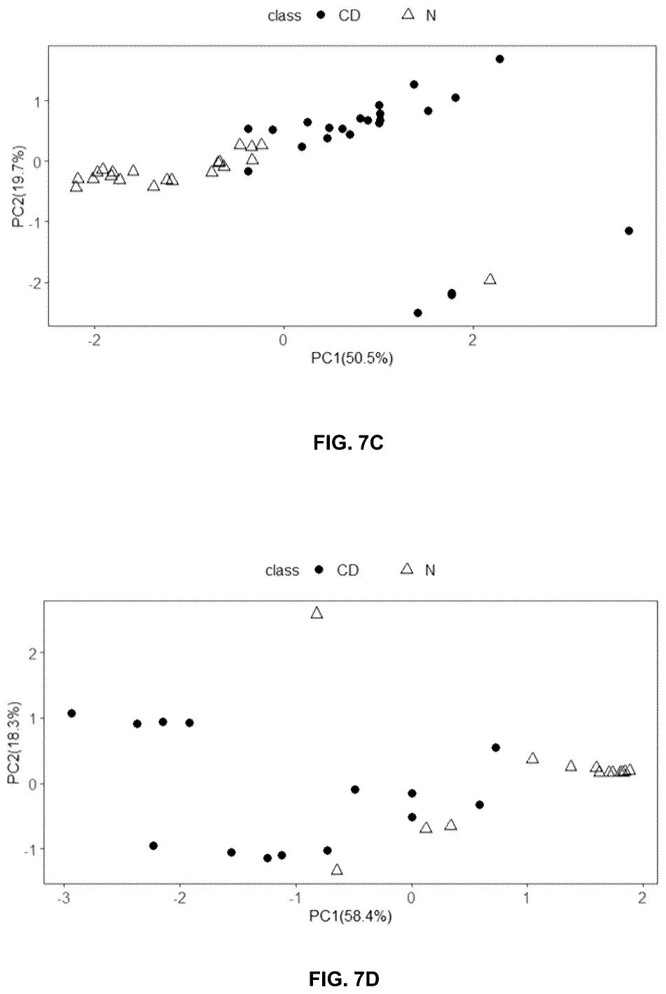

Based on the metabolites panel by targeted detection described in table 31, fold change and p value between the 21 normal and 23 CD individuals within the training set (described in table 32) were evaluated. Significantly altered serum metabolites (fold change >1.2 or <0.8, p value<0.05) were filtered out and subsequent feature selection for model construction were carried out using the LASSO algorithm. Subsequently, prediction models were constructed based on logistic regression to discriminate CD and normal individuals in the training set, achieving an AUC of 0.97 (sensitivity=95.7%, specificity=95.2%, PPV=0.96, NPV=0.95) (FIG. 7A). To further evaluate performance of this model, the performance of the N vs CD diagnostic model in the testing set was examined. This model could also yields an AUC of of 0.95 (sensitivity=92.9%, specificity=92.9%, PPV=0.93, NPV=0.93). To discriminate CD patients from normal individuals in the testing set (FIG. 7B), and PCA plots (FIG. 7C and FIG. 7D) also showed clear separations between the normal individuals and IBD patients in both training and the testing set. Significantly altered serum metabolites were listed in Table 37, Metabolites used for the N vs CD diagnostic model were listed in Table 38. In addition, using either 1 or combination of 2 or 3 metabolites listed in Table 39 could also acquire promising performances for N vs CD diagnosis. Metabolites or metabolites combinations and their respective performances were listed in table 39.

TABLE 37
List of significantly altered serum metabolites in the N vs CD

| | average | average | | |
|---|---|---|---|---|
| | abundance | abundance | | |
| | in N | in CD | | |
| Meta ID | samples | samples | Foldchange | pvalue |
| BN001 | 8183 | 4117 | 0.503 | 5.28E−05 |
| BN003 | 3104 | 1681 | 0.542 | 0.004582 |
| BN006 | 142921 | 74222 | 0.519 | 1.54E−02 |
| BN012 | 33300 | 7496 | 0.225 | 2.14E−07 |
| BN015 | 74240 | 39621 | 0.534 | 1.91E−06 |
| BN021 | 970 | 4912 | 5.07 | 0.000363 |
| BN022 | 6033 | 29741 | 4.93 | 0.00061 |
| BN023 | 301821 | 133455 | 0.442 | 8.63E−03 |
| BN028 | 42492 | 16508 | 0.389 | 0.007833 |
| BN029 | 15303992 | 2289963 | 0.15 | 1.10E−09 |
| BN030 | 885245 | 557202 | 0.629 | 6.08E−05 |
| BP003 | 55156 | 24262 | 0.44 | 0.004559 |
| BP009 | 268194 | 110931 | 0.414 | 0.002885 |
| BP013 | 17819 | 4679 | 0.263 | 1.82E−07 |
| C004 | 12081 | 159089 | 13.2 | 0.034649 |
| C006 | 68737 | 47742 | 0.695 | 0.006223 |
| C008 | 637104 | 413710 | 0.649 | 0.000127 |
| C015 | 2713 | 24252 | 8.94 | 0.000345 |
| C017 | 872740 | 509860 | 0.584 | 0.000103 |
| C019 | 35887 | 14160 | 0.395 | 2.01E−05 |
| C027 | 127794 | 48275 | 0.378 | 1.65E−06 |
| C031 | 102535209 | 77003942 | 0.751 | 6.39E−04 |
| C033 | 1310908 | 991196 | 0.756 | 0.012253 |
| C120 | 752 | 11482 | 15.3 | 6.16E−05 |
| C132 | 282499 | 167166 | 0.592 | 0.004443 |
| C145 | 326344 | 180025 | 0.552 | 0.004194 |
| C147 | 95155 | 51557 | 0.542 | 0.00148 |
| C150 | 46113 | 55803 | 1.21 | 0.006714 |
| DS01 | 311013 | 1774109 | 5.7 | 0.000475 |
| DS02 | 487702 | 6394961 | 13.1 | 4.16E−05 |
| DS10 | 267758 | 1197514 | 4.47 | 0.003601 |
| DS11 | 906527 | 2636532 | 2.91 | 0.009135 |
| X004 | 4613023 | 2914182 | 0.632 | 0.045532 |
| X006 | 208696 | 838347 | 4.02 | 0.020417 |
| X011 | 87860 | 63155 | 0.719 | 0.028248 |
| X016 | 222810 | 113005 | 0.507 | 0.01748 |
| X023 | 3934149 | 1888209 | 0.48 | 0.024163 |
| X036 | 1129 | 3271 | 2.9 | 0.025905 |
| X055 | 7245417 | 4975959 | 0.687 | 0.004099 |
| X066 | 6309371 | 4178876 | 0.662 | 0.001217 |
| X166 | 131134 | 72237 | 0.551 | 0.002014 |
| X278 | 600761 | 432539 | 0.72 | 0.049473 |
| X280 | 5966 | 3456 | 0.579 | 0.007032 |
| X281 | 38561 | 15909 | 0.413 | 6.85E−05 |
| X285 | 14329935 | 10287224 | 0.718 | 0.015646 |
| X401 | 16246 | 9220 | 0.568 | 0.000331 |
| X403 | 11968117 | 7530034 | 0.629 | 0.020306 |
| X407 | 3268 | 469 | 0.144 | 0.025973 |
| X408 | 13573 | 4058 | 0.299 | 6.82E−05 |
| X409 | 3019 | 1769 | 0.586 | 0.00844 |
| X411 | 34827 | 10163 | 0.292 | 2.89E−06 |
| X508 | 14027 | 247788 | 17.7 | 1.38E−05 |
| X513 | 23482 | 14573 | 0.621 | 4.77E−02 |
| X666 | 34338 | 56314 | 1.64 | 0.02665 |
| X679 | 31338927 | 40113826 | 1.28 | 4.30E−07 |
| X665 | 4581080 | 3568661 | 0.779 | 0.002942 |
| X659 | 592748 | 9662 | 0.0163 | 0.0004322 |
| X667 | 10355136 | 890542 | 0.086 | 1.85E−05 |
| X660 | 4065126 | 5975735 | 1.47 | 2.58E−14 |
| X657 | 2221071 | 2687496 | 1.21 | 0.04999 |
| X661 | 87102 | 63759 | 0.732 | 0.00102 |
| X662 | 44101 | 63947 | 1.45 | 1.17E−10 |
| X663 | 1225631 | 741507 | 0.605 | 0.03548 |

TABLE 38
List of metabolites involved in the N vs CD diagnostic model.

| NO | Meta ID |
|---|---|
| 1 | BN012 |
| 2 | BN021 |
| 3 | BN029 |
| 4 | BP013 |
| 5 | C004 |
| 6 | C006 |
| 7 | C015 |
| 8 | C027 |
| 9 | C031 |
| 10 | DS02 |
| 11 | X036 |
| 12 | X280 |
| 13 | X508 |
| 14 | X666 |
| 15 | X679 |
| 16 | X665 |
| 17 | X659 |
| 18 | X667 |
| 19 | X660 |
| 20 | X657 |
| 21 | X661 |
| 22 | X662 |
| 23 | X663 |
| / | / |

TABLE 39
List of Metabolites or metabolites combinations and their
respective performances in the N vs CD diagnostic model.

| Meta ID | AUC | Sens | Spec | Meta ID | AUC | Sens | Spec |
|---|---|---|---|---|---|---|---|
| BN029 | 0.98 | 0.98 | 0.96 | C019, BP012 | 0.85 | 0.79 | 0.88 |
| C004 | 0.91 | 0.84 | 0.95 | BN001, BN029, X082 | 0.97 | 0.95 | 0.96 |
| C019 | 0.85 | 0.83 | 0.86 | BN029, C027, X082 | 0.97 | 0.97 | 0.95 |
| C027 | 0.84 | 0.81 | 0.85 | C146, C150, X508 | 0.93 | 0.90 | 0.93 |
| X508 | 0.92 | 0.89 | 0.97 | BN001, C150, X004 | 0.86 | 0.82 | 0.87 |
| BN029, X082 | 0.97 | 0.97 | 0.96 | X508, C036, BN001 | 0.92 | 0.90 | 0.95 |
| C146, X508 | 0.92 | 0.88 | 0.97 | C019, C036, C150 | 0.86 | 0.80 | 0.92 |
| X508, C036 | 0.91 | 0.89 | 0.96 | BN001, C004, C027 | 0.96 | 0.91 | 0.95 |
| C019, C150 | 0.86 | 0.81 | 0.93 | C019, X004, X403 | 0.83 | 0.85 | 0.84 |
| C004, C027 | 0.93 | 0.83 | 0.97 | DS02, C009, C027 | 0.90 | 0.87 | 0.89 |
| X004, C019 | 0.84 | 0.83 | 0.87 | BP012, C027, C150 | 0.84 | 0.84 | 0.85 |
| C009, DS02 | 0.87 | 0.83 | 0.88 | BN029, C146, C150 | 0.97 | 0.97 | 0.96 |
| BN029, C150 | 0.98 | 0.98 | 0.96 | BN029, C027, C147 | 0.97 | 0.97 | 0.96 |
| C147, C027 | 0.88 | 0.86 | 0.84 | BN029, DS02, X004 | 0.97 | 0.98 | 0.95 |
| BN029, X004 | 0.98 | 0.97 | 0.96 | BP012, C019, DS02 | 0.95 | 0.90 | 1.00 |
| C019, BP012 | 0.85 | 0.79 | 0.88 | C019, BP012 | 0.85 | 0.79 | 0.88 |

### III. Diagnostic Model to Discriminate Different Subtypes of IBD: UC Vs CD Patients.

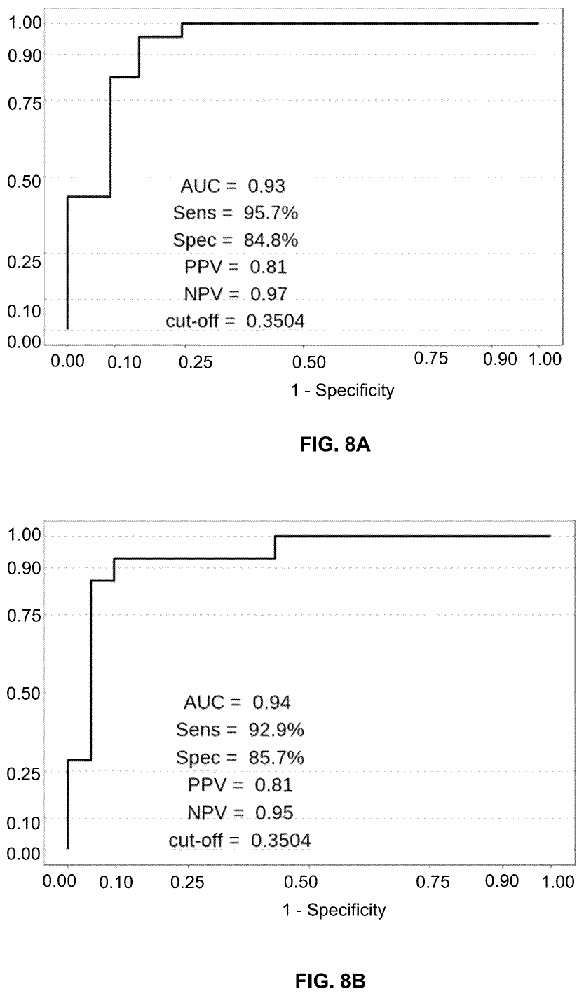

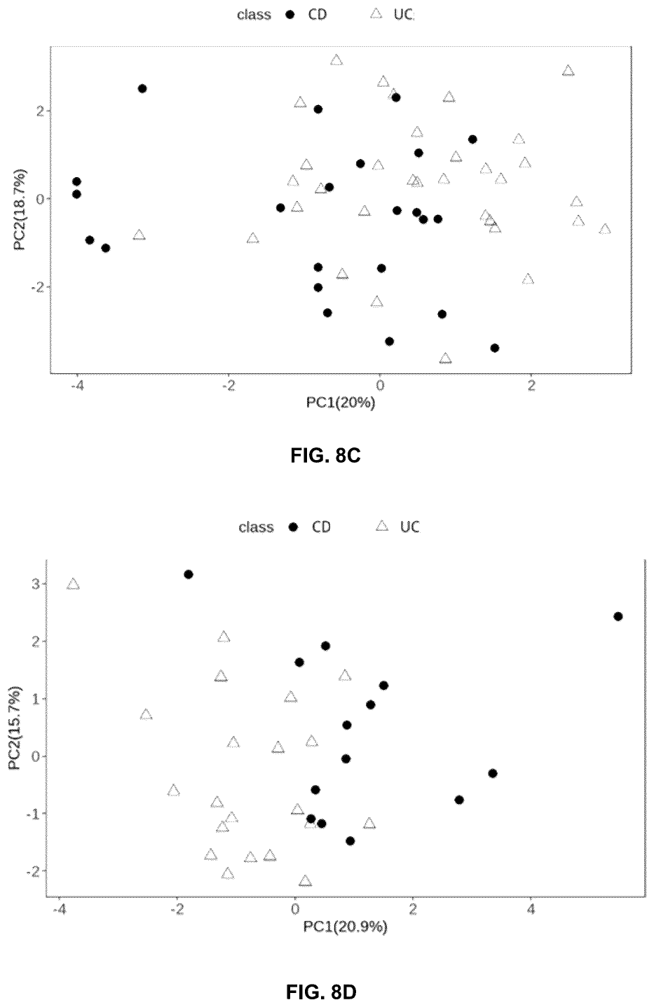

Based on the metabolites panel by targeted detection described in table 31, fold change and p value between the 33 UC and 23 CD individuals within the training set (described in table 32) was calculated. Significantly altered serum metabolites (fold change >1.2 or <0.8, p value<0.05) were filtered out and subsequent feature selection for model construction were carried out using the LASSO algorithm. Subsequently, prediction models were constructed based on logistic regression to discriminate CD and UC individuals in the training set, achieving an AUC of 0.93 (sensitivity=95.7%, specificity=84.8%, PPV=0.81, NPV=0.97) (FIG. 8A). To further evaluate performance of this model, the performance of the UC vs CD diagnostic model in the testing set was evaluated. This model could also yields an AUC of 0.94 (sensitivity=92.9%, specificity=85.7%, PPV=0.81, NPV=0.95). To discriminate CD patients from UC patients in the testing set (FIG. 8B), and PCA plots (FIG. 8C and FIG. 8D) also showed clear separations between the normal individuals and IBD patients in both training and the testing set. Significantly altered serum metabolites were listed in Table 40, Metabolites used for the UC vs CD diagnostic model were listed in Table 41. In addition, using either 1 or combination of 2 or 3 metabolites listed in Table 42 could also acquire promising performances for UC vs CD diagnosis. Metabolites or metabolites combinations and their respective performances were listed in table 42.

TABLE 40
List of significantly altered serum metabolites in the UC vs CD

| | average | average | | |
|---|---|---|---|---|
| | abundance | abundance | | |
| | in UC | in CD | | |
| Meta ID | samples | samples | foldchange | pvalue |
| BN012 | 19727 | 7496 | 0.38 | 0.024424 |
| BN025 | 43622 | 22521 | 0.52 | 0.005271 |
| BN030 | 742638 | 557202 | 0.75 | 0.00483 |
| BP003 | 35653 | 24262 | 0.68 | 0.045304 |
| BP006 | 2403 | 1144 | 0.48 | 0.029915 |
| BP010 | 136450 | 87050 | 0.64 | 0.003533 |
| BP012 | 285704 | 192972 | 0.68 | 0.020551 |
| C006 | 73406 | 47742 | 0.65 | 0.001008 |
| C008 | 578910 | 413710 | 0.71 | 0.000634 |
| C009 | 818418 | 605629 | 0.74 | 0.017168 |
| C011 | 2250936 | 1793122 | 0.8 | 0.016408 |
| C043 | 200107 | 97189 | 0.49 | 0.02182 |
| C112 | 18916 | 35072 | 1.85 | 0.027143 |
| C122 | 14969 | 9069 | 0.61 | 0.017555 |
| C124 | 327004 | 204768 | 0.63 | 0.004693 |
| C128 | 33425 | 22765 | 0.68 | 0.000389 |
| C129 | 34072 | 19962 | 0.59 | 0.003453 |
| C146 | 2147826 | 2922200 | 1.36 | 0.026514 |
| DS04 | 394572 | 187444 | 0.48 | 0.017835 |
| DS05 | 3251447 | 1637598 | 0.5 | 0.022024 |
| DS07 | 4574 | 22246 | 4.86 | 0.037868 |
| DS11 | 1068781 | 2636532 | 2.47 | 0.018284 |
| X055 | 3708044 | 4975959 | 1.34 | 0.043106 |
| X082 | 1223729 | 3862 | 0 | 0.004513 |
| X292 | 1791990 | 5401750 | 3.01 | 0.012143 |
| X407 | 2427 | 469 | 0.19 | 0.049584 |
| X411 | 21747 | 10163 | 0.47 | 0.012428 |
| X412 | 51096 | 37300 | 0.73 | 0.00815 |
| X515 | 11758 | 8011 | 0.68 | 0.016435 |
| X676 | 2312750 | 2960321 | 1.28 | 0.0492 |
| X653 | 250021 | 81757 | 0.327 | 4.30E−05 |
| X659 | 7668 | 9662 | 1.26 | 0.01642 |
| X665 | 4888577 | 3568661 | 0.73 | 0.02996 |
| X652 | 22374419 | 14364377 | 0.642 | 0.000256 |
| X658 | 162355 | 206191 | 1.27 | 0.04083 |
| X673 | 42983 | 61465 | 1.43 | 0.03155 |
| X670 | 562634 | 720172 | 1.28 | 0.04783 |
| X672 | 472723 | 576722 | 1.22 | 0.04759 |
| X675 | 26815647 | 33251402 | 1.24 | 0.04996 |

TABLE 41
List of metabolites involved in the UC vs CD diagnostic model.

| NO | Meta ID | | | | | | |
|---|---|---|---|---|---|---|---|
| 1 | BN012 | 8 | DS04 | 14 | X411 | 20 | X652 |
| 2 | BN025 | 9 | DS05 | 15 | X515 | 21 | X658 |
| 3 | BP003 | 10 | DS07 | 16 | X676 | 22 | X673 |
| 4 | C008 | 11 | DS11 | 17 | X653 | 23 | X670 |
| 5 | C112 | 12 | X082 | 18 | X659 | 24 | X672 |
| 6 | C128 | 13 | X407 | 19 | X665 | 25 | X675 |
| 7 | C146 | / | / | / | / | / | / |

TABLE 42
List of Metabolites or metabolites combinations and their
respective performances in the UC vs CD diagnostic model.

| Meta ID | AUC | Sens | Spec | Meta ID | AUC | Sens | Spec |
|---|---|---|---|---|---|---|---|
| DS11 | 0.72 | 0.54 | 0.88 | BP003, DS11, X407 | 0.77 | 0.66 | 0.84 |
| X082 | 0.73 | 0.74 | 0.71 | C008, DS04, DS07 | 0.77 | 0.59 | 0.88 |
| C128 | 0.71 | 0.56 | 0.89 | C112, C146, X082 | 0.84 | 0.78 | 0.85 |
| BN012, X082 | 0.76 | 0.65 | 0.82 | C112, C146, X515 | 0.79 | 0.63 | 0.89 |
| BN025, X082 | 0.76 | 0.66 | 0.84 | C112, DS11, X407 | 0.82 | 0.67 | 0.9 |
| BN012, DS11, X407 | 0.8 | 0.73 | 0.85 | C112, DS11, X411 | 0.81 | 0.73 | 0.83 |
| BN025, C008, DS04 | 0.76 | 0.61 | 0.86 | C128, DS07, X082 | 0.81 | 0.66 | 0.9 |
| BN025, DS04, DS11 | 0.83 | 0.8 | 0.85 | C146, DS05, X082 | 0.79 | 0.75 | 0.81 |
| BN025, DS05, DS11 | 0.79 | 0.65 | 0.89 | DS07, DS11, X082 | 0.83 | 0.69 | 0.91 |
| BN025, DS07, X082 | 0.78 | 0.67 | 0.84 | / | / | / | / |

### IV. Diagnostic Model to Discriminate Non-IBD and IBD Patients Including Both UC and CD

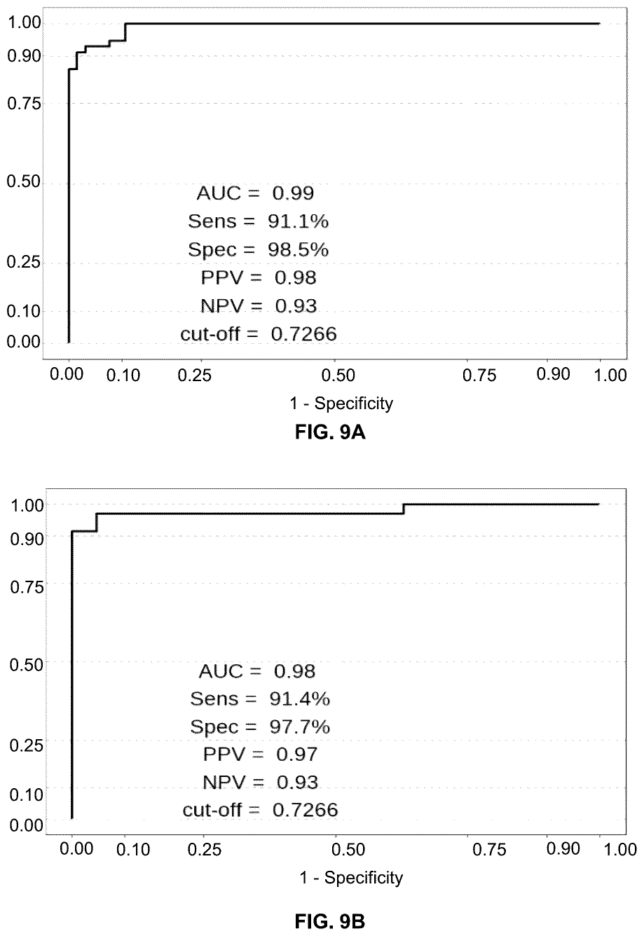

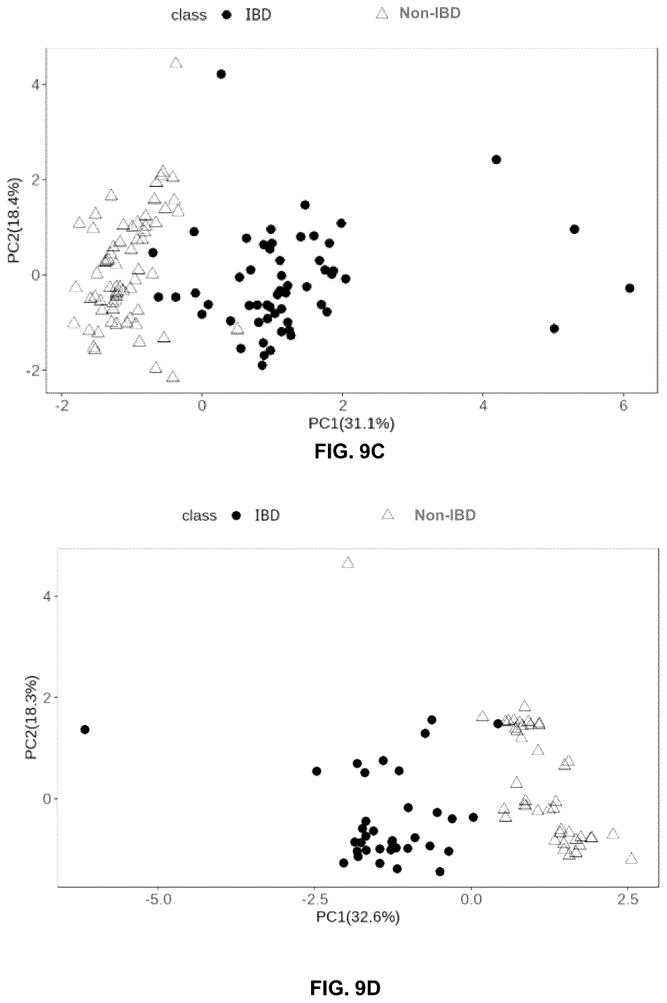

Next, both UC and CD patients were integrated into the IBD group, and the model was further evaluated to discriminate between non-IBD individuals and all IBD patients. Based on the metabolites panel by targeted detection described in table 31, fold change and p value were calculated between the non-IBD (including the 21 normal, 29 adenoma and 16 non-adenoma polyps) and 56 IBD individuals within the training set (described in table 32). Significantly altered serum metabolites (fold change >1.2 or <0.8, p value<0.05) were filtered out and subsequent feature selection for model construction were carried out using the LASSO algorithm. Subsequently, prediction models were constructed based on logistic regression to discriminate IBD and non-IBD individuals in the training set, achieving an AUC of 0.99 (sensitivity=91.1%, specificity=98.5%, PPV=0.98, NPV=0.93) (FIG. 9A). To further evaluate performance of this model, the performance of the non-IBD vs IBD diagnostic model in the testing set was evaluated. This model could also yields an AUC of 0.98 (sensitivity=91.4%, specificity=97.7%, PPV=0.97, NPV=0.93) to discriminate IBD patients from non-IBD individuals in the testing set (FIG. 9B), and PCA plots (FIG. 9C and FIG. 9D) also showed clear separations between the non-IBD individuals and IBD patients in both training and the testing set. Significantly altered serum metabolites were listed in Table 43, Metabolites used for the non-IBD vs IBD diagnostic model were listed in Table 44. In addition, using either 1 or combination of 2 or 3 metabolites listed in Table 45 could also acquire promising performances for diagnosis of non-IBD vs all IBD patient. Metabolites or metabolites combinations and their respective performances were listed in table 45.

TABLE 43
List of significantly altered serum
metabolites in the non-IBD vs IBD

| | average | average | | |
|---|---|---|---|---|
| | abundance | abundance in | | |
| Meta ID | in N samples | IBD samples | foldchange | pvalue |
| BN001 | 14069 | 4404 | 0.313 | 0.00058408 |
| BN003 | 3559 | 2114 | 0.594 | 0.000035181 |
| BN004 | 1124 | 659 | 0.586 | 0.017982 |
| BN006 | 152364 | 100256 | 0.658 | 0.0075865 |
| BN012 | 39615 | 14737 | 0.372 | 1.56E−07 |
| BN013 | 1407 | 601 | 0.427 | 9.46E−07 |
| BN015 | 83596 | 40293 | 0.482 | 1.48E−20 |
| BN016 | 92709 | 112178 | 1.21 | 0.029618 |
| BN017 | 10442896 | 4312916 | 0.413 | 0.000085268 |
| BN020 | 22985 | 196981 | 8.57 | 0.024831 |
| BN021 | 1446 | 4136 | 2.86 | 0.0029601 |
| BN022 | 8663 | 25296 | 2.92 | 0.0020289 |
| BN023 | 359449 | 150969 | 0.42 | 5.66E−10 |
| BN027 | 1785518 | 1415916 | 0.793 | 0.0001075 |
| BN028 | 51570 | 14130 | 0.274 | 1.42E−10 |
| BN029 | 17170418 | 2764437 | 0.161 | 1.11E−27 |
| BN030 | 940860 | 667070 | 0.709 | 4.87E−07 |
| BP002 | 744715 | 70897 | 0.0952 | 5.5535E−06 |
| BP003 | 64940 | 31041 | 0.478 | 4.33E−08 |
| BP007 | 88417 | 54465 | 0.616 | 1.3371E−06 |
| BP009 | 326950 | 161513 | 0.494 | 0.00013217 |
| BP011 | 1911973 | 2313487 | 1.21 | 0.024598 |
| BP013 | 18295 | 5708 | 0.312 | 1.29E−21 |
| C004 | 11343 | 141788 | 12.5 | 0.00012294 |
| C008 | 678966 | 511940 | 0.754 | 9.35E−07 |
| C016 | 33674 | 12459 | 0.37 | 0.029032 |
| C017 | 987903 | 497903 | 0.504 | 2.69E−16 |
| C019 | 38092 | 14818 | 0.389 | 5.35E−15 |
| C021 | 461033 | 339781 | 0.737 | 0.027614 |
| C026 | 17152029 | 12057876 | 0.703 | 0.000031655 |
| C027 | 137405 | 44382 | 0.323 | 3.75E−23 |
| C031 | 104882551 | 78557031 | 0.749 | 1.08E−09 |
| C033 | 1394122 | 1055350 | 0.757 | 8.32E−07 |
| C035 | 4909210 | 3495358 | 0.712 | 0.00033583 |
| C041 | 4560 | 2016 | 0.442 | 0.00027917 |
| C043 | 75292 | 158113 | 2.1 | 0.0019142 |
| C102 | 440405 | 37875 | 0.086 | 3.21E−09 |
| C110 | 93075 | 52680 | 0.566 | 2.14E−07 |
| C112 | 49783 | 25489 | 0.512 | 0.018773 |
| C116 | 4654 | 3295 | 0.708 | 0.03414 |
| C119 | 28411 | 34377 | 1.21 | 0.046446 |
| C120 | 1002 | 25050 | 25 | 0.047465 |
| C131 | 6842103 | 3893157 | 0.569 | 6.0812E−06 |
| C132 | 277837 | 169203 | 0.609 | 4.20E−09 |
| C135 | 776438 | 92396 | 0.119 | 1.5041E−06 |
| C136 | 59169 | 33371 | 0.564 | 4.03E−07 |
| C137 | 100393 | 66761 | 0.665 | 0.00056099 |
| C139 | 83186 | 63804 | 0.767 | 0.020904 |
| C144 | 909 | 2663 | 2.93 | 0.00864 |
| C145 | 344117 | 179973 | 0.523 | 1.41E−11 |
| C146 | 4217150 | 2462816 | 0.584 | 9.37E−07 |
| C147 | 91281 | 53491 | 0.586 | 1.47E−08 |
| C148 | 1843518 | 978908 | 0.531 | 1.47E−08 |
| C149 | 539128 | 355824 | 0.66 | 4.4086E−06 |
| C150 | 45467 | 56379 | 1.24 | 0.00071839 |
| DS01 | 349551 | 2719507 | 7.78 | 0.020421 |
| DS02 | 1042548 | 5150187 | 4.94 | 0.000012595 |
| DS04 | 123422 | 309789 | 2.51 | 0.00045925 |
| DS05 | 1283390 | 2592448 | 2.02 | 0.0026711 |
| DS10 | 294982 | 1563405 | 5.3 | 0.0076913 |
| DS11 | 737299 | 1703161 | 2.31 | 0.0037048 |
| X004 | 5807790 | 2212768 | 0.381 | 2.65E−12 |
| X006 | 138205 | 562494 | 4.07 | 0.0004319 |
| X013 | 289080 | 169690 | 0.587 | 0.0016811 |
| X016 | 235869 | 154258 | 0.654 | 0.0082046 |
| X023 | 7440407 | 3065448 | 0.412 | 3.73E−07 |
| X024 | 383290 | 147567 | 0.385 | 0.0092432 |
| X055 | 7158614 | 4223582 | 0.59 | 5.47E−12 |
| X066 | 6441194 | 4135247 | 0.642 | 2.47E−11 |
| X082 | 399 | 730170 | 1830 | 0.0047517 |
| X154 | 58396 | 40527 | 0.694 | 0.011802 |
| X160 | 1969860 | 2383531 | 1.21 | 0.027752 |
| X166 | 142594 | 72580 | 0.509 | 3.96E−12 |
| X183 | 155476 | 89554 | 0.576 | 2.0648E−06 |
| X188 | 6752089 | 3625872 | 0.537 | 0.000029034 |
| X278 | 677204 | 367722 | 0.543 | 1.61E−10 |
| X280 | 5245 | 3771 | 0.719 | 0.0023204 |
| X281 | 41329 | 16532 | 0.4 | 8.44E−14 |
| X285 | 15109924 | 9020625 | 0.597 | 3.75E−10 |
| X286 | 1513896 | 962838 | 0.636 | 2.0666E−06 |
| X289 | 253683 | 168699 | 0.665 | 5.13E−07 |
| X292 | 1660582 | 3254741 | 1.96 | 0.024228 |
| X293 | 92640 | 65589 | 0.708 | 0.030974 |
| X401 | 17511 | 9158 | 0.523 | 1.09E−09 |
| X403 | 14477867 | 5935925 | 0.41 | 3.64E−14 |
| X407 | 3773 | 1630 | 0.432 | 0.040185 |
| X408 | 17186 | 6239 | 0.363 | 1.06E−07 |
| X409 | 3100 | 1872 | 0.604 | 0.000031178 |
| X411 | 40003 | 17041 | 0.426 | 8.09E−09 |
| X508 | 4664 | 228070 | 48.9 | 2.86E−10 |
| X513 | 26490 | 17483 | 0.66 | 0.00086 |
| X519 | 4249 | 2639 | 0.621 | 0.00076686 |
| X525 | 40289 | 25422 | 0.631 | 0.0032362 |
| X664 | 908056 | 590236 | 0.65 | 0.003076 |
| X657 | 7998501 | 2687496 | 0.336 | 0.00139 |
| X682 | 5096 | 6370 | 1.25 | 0.0268 |
| X667 | 6058107 | 890542 | 0.147 | 0.04714 |
| X677 | 3909161 | 2247768 | 0.575 | 0.0003429 |
| X653 | 156024 | 81757 | 0.524 | 0.0001427 |
| X684 | 216329 | 263921 | 1.22 | 0.01103 |
| X678 | 61284 | 48169 | 0.786 | 0.04626 |
| X681 | 8808110 | 7002447 | 0.795 | 0.03099 |
| X686 | 22155031 | 15663607 | 0.707 | 0.01566 |

TABLE 44
List of metabolites involved in the
non-IBD vs IBD diagnostic model.

| NO | Meta ID |
|---|---|
| 1 | BN017 |
| 2 | BN021 |
| 3 | BN029 |
| 4 | BP002 |
| 5 | BP011 |
| 6 | BP013 |
| 7 | C004 |
| 8 | C008 |
| 9 | C019 |
| 10 | C027 |
| 11 | C119 |
| 12 | C147 |
| 13 | C148 |
| 14 | DS02 |
| 15 | DS04 |
| 16 | X024 |
| 17 | X082 |
| 18 | X285 |
| 19 | X293 |
| 20 | X403 |
| 21 | X407 |
| 22 | X508 |
| 23 | X664 |
| 24 | X657 |
| 25 | X682 |
| 26 | X667 |
| 27 | X677 |
| 28 | X653 |
| 29 | X684 |
| 30 | X678 |
| 31 | X681 |
| 32 | X686 |

TABLE 45
List of Metabolites or metabolites combinations and their respective
performances in the non-IBD vs IBD diagnostic model.

| Meta ID | AUC | Sens | Spec | Meta ID | AUC | Sens | Spec |
|---|---|---|---|---|---|---|---|
| BN029 | 0.97 | 0.91 | 0.97 | C008, X285 | 0.77 | 0.61 | 0.88 |
| BP013 | 0.93 | 0.86 | 0.91 | C027, X024 | 0.89 | 0.81 | 0.87 |
| C004 | 0.89 | 0.78 | 0.94 | BN029, C027 | 0.98 | 0.93 | 0.96 |
| C008 | 0.7 | 0.51 | 0.88 | BP013, C119 | 0.95 | 0.87 | 0.95 |
| C019 | 0.86 | 0.76 | 0.88 | BN017, BP002, C004 | 0.9 | 0.76 | 0.95 |
| C027 | 0.89 | 0.79 | 0.89 | BP013, DS04, X407 | 0.94 | 0.85 | 0.95 |
| C147 | 0.74 | 0.55 | 0.88 | BP011, C004, C147 | 0.92 | 0.79 | 0.96 |
| C148 | 0.77 | 0.59 | 0.9 | BN029, C119, X293 | 0.98 | 0.92 | 0.98 |
| X285 | 0.76 | 0.6 | 0.88 | C027, C148, X407 | 0.92 | 0.85 | 0.89 |
| X403 | 0.83 | 0.71 | 0.89 | C004, C027, X293 | 0.94 | 0.85 | 0.95 |
| X508 | 0.9 | 0.83 | 0.99 | BN021, DS04, X403 | 0.87 | 0.87 | 0.86 |
| BP002, C004 | 0.9 | 0.78 | 0.94 | BP013, X293, X407 | 0.92 | 0.83 | 0.93 |
| BP011, C147 | 0.77 | 0.62 | 0.88 | C019, C027, C119 | 0.9 | 0.8 | 0.9 |
| C027, X407 | 0.89 | 0.77 | 0.91 | BN021, X024, X082 | 0.78 | 0.57 | 0.93 |
| C027, X293 | 0.89 | 0.76 | 0.91 | BN029, BP011, C027 | 0.98 | 0.93 | 0.97 |
| BN021, X403 | 0.87 | 0.86 | 0.86 | C019, C119, X508 | 0.95 | 0.88 | 0.97 |
| BP013, X407 | 0.92 | 0.84 | 0.91 | BP011, C027, DS04 | 0.9 | 0.79 | 0.9 |
| C019, C119 | 0.87 | 0.74 | 0.92 | BN017, C119, DS04 | 0.76 | 0.59 | 0.9 |
| BN029, BP011 | 0.97 | 0.92 | 0.97 | BN017, C008, X285 | 0.77 | 0.59 | 0.88 |
| C019, X508 | 0.95 | 0.89 | 0.97 | BN017, C027, X024 | 0.89 | 0.81 | 0.87 |
| BN017, DS04 | 0.76 | 0.55 | 0.91 | BN021, BP013, C119 | 0.96 | 0.88 | 0.95 |

### V. Diagnostic Model to Discriminate Colorectal Polyps (Adenoma and Non-Adenoma) and IBD Patients.

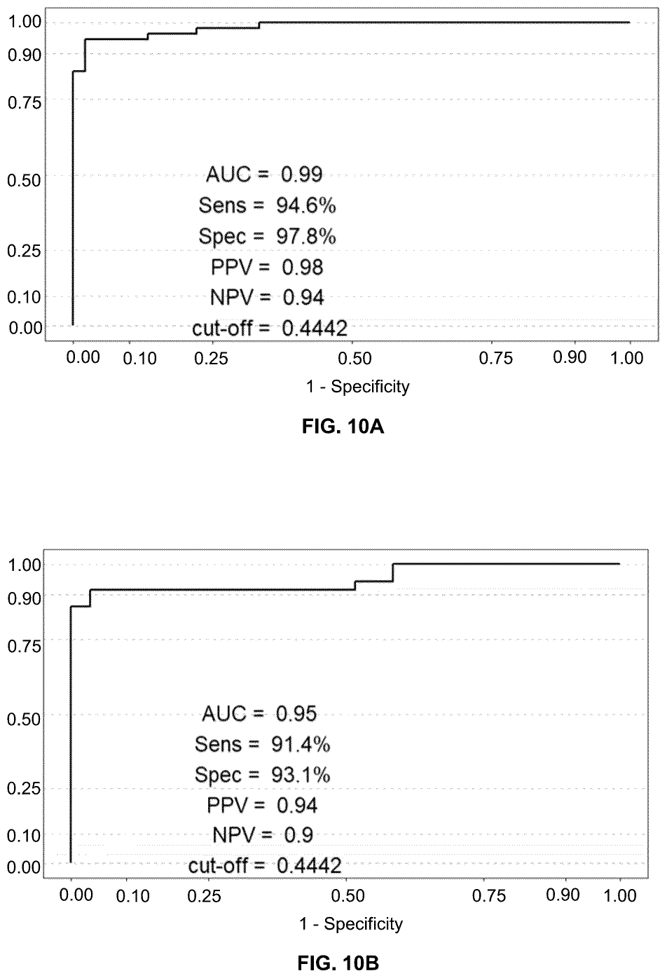

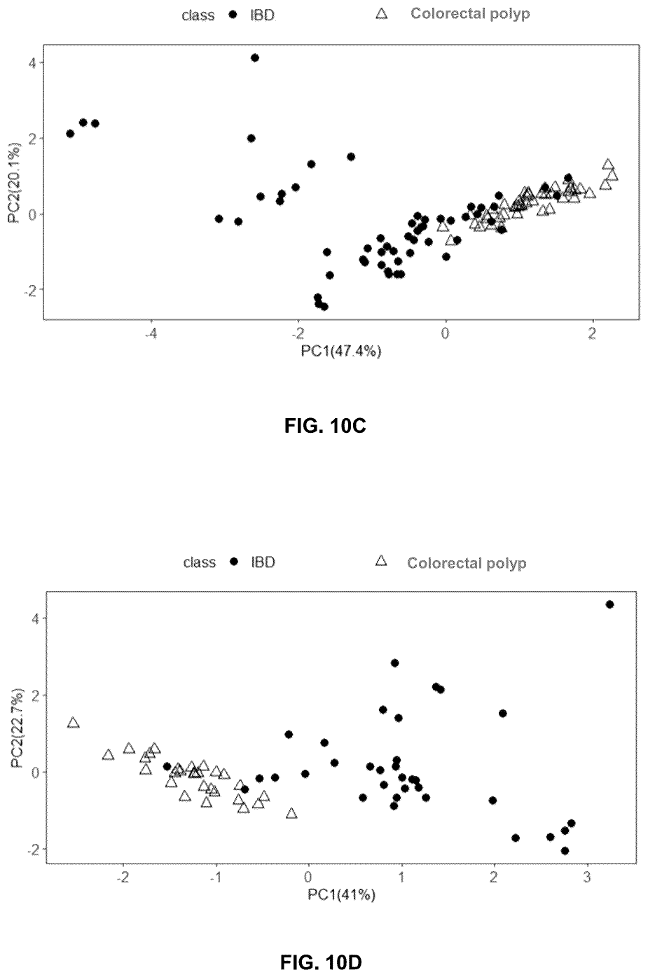

Diagnosis of IBD (for both Crohn's disease and ulcerative colitis) requires the combination of colonoscopy examination and histological examination of the biopsies. In the current calculation, colorectal polyps were found in more than 30% in individuals under colonoscopy test, making it an important interfering disease for IBD diagnosis. These polyps might attribute to inflammation, hyperplastic or adenoma. In this modeling cohort, 48 adenoma and 26 non-adenoma polyps patients were enrolled and the efficiency to discriminate them from IBD patients were evaluated. Based on the metabolites panel by targeted detection described in table 31, fold change and p value between the 45 colorectal polyps and 56 IBD individuals within the training set (described in table 32) were also evaluated. Significantly altered serum metabolites (fold change >1.2 or <0.8, p value<0.05) were filtered out and subsequent feature selection for model construction were carried out using the LASSO algorithm. Subsequently, prediction models were constructed based on logistic regression to discriminate IBD and colorectal polyps individuals in the training set, achieving an AUC of 0.99 (sensitivity=94.6%, specificity=97.8%, PPV=0.98, NPV=0.94) (FIG. 10A). To further evaluate performance of this model, the performance of the normal vs IBD diagnostic model in the testing set was evaluated. This model could also yields an AUC of 0.95 (sensitivity=91.4%, specificity=93.1%, PPV=0.94, NPV=0.9) to discriminate IBD patients from normal individuals in the testing set (FIG. 10B), and PCA plots (FIG. 10C and FIG. 10D) also showed clear separations between the normal individuals and IBD patients in both training and the testing set, and these findings further proved the specificity of certain disease related gut microbiome associated serum metabolites on diagnosis of the respective disease. Significantly altered serum metabolites were listed in Table 46, Metabolites used for the Normal vs IBD diagnostic model were listed in Table 47. In addition, using either 1 or combination of 2 or 3 metabolites listed in Table 48 could also acquire promising performances for colorectal polyps vs IBD diagnosis. Metabolites or metabolites combinations and their respective performances were listed in table 48.

TABLE 46
List of significantly altered serum metabolites
in the colorectal polyps vs IBD

| | average | average | | |
|---|---|---|---|---|
| | abundance in | abundance | | |
| | colorectal polyps | in IBD | | |
| Meta ID | samples | samples | foldchange | pvalue |
| BN001 | 16853 | 4401 | 0.261 | 0.002445 |
| BN003 | 3774 | 2113 | 0.56 | 1.74E−05 |
| BN006 | 156830 | 100293 | 0.64 | 0.006452 |
| BN012 | 42602 | 14754 | 0.346 | 1.01E−06 |
| BN015 | 88020 | 40295 | 0.458 | 6.76E−16 |
| BN016 | 95069 | 74724 | 0.786 | 0.027698 |
| BN017 | 12778641 | 4313401 | 0.338 | 6.98E−05 |
| BN020 | 28509 | 196973 | 6.91 | 0.031808 |
| BN021 | 1671 | 4132 | 2.47 | 0.007199 |
| BN022 | 9908 | 25299 | 2.55 | 0.004887 |
| BN023 | 386706 | 150939 | 0.39 | 3.01E−10 |
| BN027 | 1841006 | 1416403 | 0.769 | 5.42E−05 |
| BN028 | 55864 | 14112 | 0.253 | 6.45E−09 |
| BN029 | 18053187 | 2760277 | 0.153 | 6.83E−20 |
| BN030 | 967165 | 667241 | 0.69 | 5.19E−07 |
| BN035 | 4146 | 6271 | 1.51 | 0.047909 |
| BP002 | 956950 | 70921 | 0.0741 | 1.16E−05 |
| BP003 | 69567 | 31022 | 0.446 | 3.85E−08 |
| BP007 | 89244 | 54442 | 0.61 | 3.79E−06 |
| BP009 | 354740 | 161523 | 0.455 | 4.61E−05 |
| BP013 | 18521 | 5701 | 0.308 | 5.65E−18 |
| C004 | 10994 | 141859 | 12.9 | 0.00012 |
| C008 | 698766 | 511741 | 0.732 | 2.49E−07 |
| C016 | 36657 | 12447 | 0.34 | 0.021314 |
| C017 | 1042372 | 498289 | 0.478 | 8.03E−15 |
| C019 | 39136 | 14834 | 0.379 | 5.56E−13 |
| C021 | 464566 | 339133 | 0.73 | 0.034452 |
| C026 | 17601600 | 13777250 | 0.783 | 1.85E−05 |
| C027 | 141951 | 44315 | 0.312 | 1.38E−18 |
| C031 | 105992781 | 78434658 | 0.74 | 1.05E−09 |
| C032 | 58049 | 42866 | 0.738 | 0.008093 |
| C033 | 1433480 | 1055522 | 0.736 | 2.27E−07 |
| C035 | 5094667 | 3985108 | 0.782 | 3.14E−05 |
| C041 | 4160 | 2014 | 0.484 | 1.29E−07 |
| C043 | 75284 | 158261 | 2.1 | 0.003401 |
| C102 | 492415 | 37854 | 0.0769 | 4.46E−09 |
| C110 | 94196 | 52665 | 0.559 | 6.52E−07 |
| C112 | 49308 | 25485 | 0.517 | 0.02344 |
| C116 | 5001 | 3293 | 0.659 | 0.002923 |
| C120 | 1121 | 25053 | 22.3 | 0.047973 |
| C131 | 6992934 | 3891201 | 0.556 | 1.23E−05 |
| C132 | 275632 | 169173 | 0.614 | 5.54E−08 |
| C135 | 911544 | 92730 | 0.102 | 6.98E−06 |
| C136 | 59841 | 33353 | 0.557 | 8.42E−07 |
| C137 | 101514 | 66739 | 0.657 | 0.000313 |
| C139 | 85291 | 63794 | 0.748 | 0.005937 |
| C144 | 863 | 2662 | 3.08 | 0.001477 |
| C145 | 352523 | 179961 | 0.51 | 2.06E−10 |
| C146 | 4171736 | 2462682 | 0.59 | 7.12E−06 |
| C147 | 89448 | 53447 | 0.598 | 7.31E−07 |
| C148 | 1933631 | 978983 | 0.506 | 1.60E−08 |
| C149 | 540182 | 355877 | 0.659 | 1.88E−05 |
| C150 | 45162 | 56397 | 1.25 | 0.004424 |
| DS01 | 367778 | 2720081 | 7.4 | 0.021615 |
| DS02 | 1304975 | 5145294 | 3.94 | 6.57E−05 |
| DS04 | 130230 | 310355 | 2.38 | 0.000999 |
| DS05 | 1319877 | 2595267 | 1.97 | 0.0046 |
| DS10 | 307858 | 1562109 | 5.07 | 0.008531 |
| DS11 | 657258 | 1706218 | 2.6 | 0.00202 |
| X004 | 6372883 | 2210291 | 0.347 | 1.30E−11 |
| X006 | 104864 | 563163 | 5.37 | 0.000168 |
| X013 | 261052 | 169604 | 0.65 | 0.002161 |
| X016 | 242046 | 154233 | 0.637 | 0.00767 |
| X023 | 9098772 | 3065118 | 0.337 | 1.55E−06 |
| X024 | 486364 | 147481 | 0.303 | 0.003966 |
| X032 | 154001 | 115655 | 0.751 | 0.04739 |
| X055 | 7117559 | 4223570 | 0.593 | 1.09E−09 |
| X066 | 6503543 | 4138098 | 0.636 | 4.54E−11 |
| X082 | 391 | 727739 | 1860 | 0.004751 |
| X154 | 62335 | 40504 | 0.65 | 0.001672 |
| X166 | 148014 | 72587 | 0.49 | 2.01E−10 |
| X183 | 158107 | 89540 | 0.566 | 5.1E−06 |
| X188 | 7428119 | 3629228 | 0.489 | 4.98E−06 |
| X278 | 713360 | 367965 | 0.516 | 1.47E−09 |
| X280 | 4904 | 3774 | 0.769 | 0.015055 |
| X281 | 42638 | 16543 | 0.388 | 2.60E−12 |
| X285 | 15478838 | 9017238 | 0.583 | 2.37E−09 |
| X286 | 1548191 | 962105 | 0.621 | 7.3E−06 |
| X289 | 259716 | 168633 | 0.649 | 1.06E−06 |
| X292 | 1331828 | 3259695 | 2.45 | 0.003923 |
| X293 | 95236 | 74813 | 0.786 | 0.015348 |
| X401 | 18110 | 9152 | 0.505 | 7.40E−08 |
| X403 | 15664912 | 5928811 | 0.378 | 1.14E−13 |
| X408 | 18894 | 6241 | 0.33 | 9.23E−07 |
| X409 | 3138 | 1872 | 0.596 | 0.000196 |
| X411 | 42450 | 17037 | 0.401 | 2.68E−08 |
| X508 | 236 | 228098 | 966 | 8.35E−11 |
| X513 | 27913 | 17474 | 0.626 | 0.000149 |
| X519 | 4553 | 2638 | 0.579 | 0.000304 |
| X525 | 43292 | 25412 | 0.587 | 0.001157 |
| X668 | 4090600 | 2581169 | 0.631 | 0.03127 |
| X669 | 648720 | 412586 | 0.636 | 0.04225 |
| X674 | 5418670 | 3527554 | 0.651 | 0.0006859 |
| X685 | 13921 | 44268 | 3.18 | 0.007998 |
| X671 | 616657 | 315728 | 0.512 | 0.04946 |
| X653 | 233591 | 81757 | 0.35 | 2.99E−10 |
| X659 | 685234 | 9662 | 0.0141 | 0.06798 |

TABLE 47
List of metabolites involved in the colorectal
polyps vs IBD diagnostic model.

| NO | Meta ID |
|---|---|
| 1 | BN021 |
| 2 | BN029 |
| 3 | BN035 |
| 4 | BP002 |
| 5 | C004 |
| 6 | C017 |
| 7 | C021 |
| 8 | C027 |
| 9 | C035 |
| 10 | C102 |
| 11 | C112 |
| 12 | DS02 |
| 13 | DS04 |
| 14 | X024 |
| 15 | X082 |
| 16 | X293 |
| 17 | X403 |
| 18 | X408 |
| 19 | X411 |
| 20 | X508 |
| 21 | X668 |
| 22 | X669 |
| 23 | X674 |
| 24 | X685 |
| 25 | X671 |
| 26 | X653 |
| 27 | X659 |

TABLE 48
List of Metabolites or metabolites combinations and their respective
performances in the colorectal polyps vs IBD diagnostic model.

| Meta ID | AUC | Sens | Spec | Meta ID | AUC | Sens | Spec |
|---|---|---|---|---|---|---|---|
| BN029 | 0.98 | 0.92 | 0.98 | C102, X411 | 0.92 | 0.84 | 0.92 |
| C004 | 0.9 | 0.84 | 0.92 | BN029, X508 | 0.99 | 0.97 | 0.99 |
| C017 | 0.85 | 0.84 | 0.8 | C017, X411 | 0.89 | 0.85 | 0.87 |
| C027 | 0.9 | 0.86 | 0.87 | BN029, X293, X403 | 0.98 | 0.94 | 0.99 |
| C102 | 0.91 | 0.87 | 0.91 | BN029, X024, X293 | 0.98 | 0.94 | 0.97 |
| X403 | 0.85 | 0.79 | 0.85 | BN035, C004, C102 | 0.94 | 0.9 | 0.91 |
| X408 | 0.88 | 0.77 | 0.97 | C035, C112, X508 | 0.97 | 0.92 | 0.97 |
| X411 | 0.86 | 0.74 | 0.96 | C004, C102, X082 | 0.95 | 0.92 | 0.93 |
| X508 | 0.91 | 0.83 | 1 | BN021, C027, X408 | 0.94 | 0.88 | 0.95 |
| BN029, X293 | 0.98 | 0.92 | 0.99 | BN021, C102, X293 | 0.91 | 0.86 | 0.89 |
| BN035, C004 | 0.91 | 0.85 | 0.9 | BN029, C017, C112 | 0.98 | 0.93 | 0.97 |
| C035, X508 | 0.97 | 0.93 | 0.97 | BP002, BN021, C021 | 0.81 | 0.82 | 0.75 |
| C004, X082 | 0.93 | 0.88 | 0.92 | C017, X082, X411 | 0.91 | 0.83 | 0.93 |
| C027, X408 | 0.93 | 0.87 | 0.93 | C004, C021, DS02 | 0.91 | 0.86 | 0.93 |
| C102, X293 | 0.89 | 0.87 | 0.87 | BN035, C112, X411 | 0.84 | 0.75 | 0.91 |
| BN035, C102 | 0.89 | 0.89 | 0.88 | BN029, C112, X082 | 0.98 | 0.94 | 0.98 |
| C017, C112 | 0.86 | 0.83 | 0.81 | C102, DS04, X024 | 0.91 | 0.86 | 0.87 |
| C017, X082 | 0.89 | 0.83 | 0.88 | C021, C102, X411 | 0.91 | 0.83 | 0.91 |
| C004, DS02 | 0.92 | 0.87 | 0.91 | BN029, X082, X508 | 0.99 | 0.98 | 0.99 |
| C112, X411 | 0.86 | 0.73 | 0.94 | BN035, C017, X411 | 0.9 | 0.83 | 0.89 |
| BN029, C112 | 0.98 | 0.92 | 0.98 | C035, C102, C112 | 0.88 | 0.84 | 0.83 |
| C102, DS04 | 0.91 | 0.87 | 0.89 | / | / | / | / |

Collectively, a panel of serum metabolites biomarkers that showed close association with IBD related gut microbiome were established, and an MRM based targeted detection assay of these metabolites was developed. Based on these serum metabolites, an IBD diagnostic model and an UC/CD discrimination model were developed, providing a more accurate approach for detection of IBD patients and discrimination between UC and CD individuals.

1. A system for detecting inflammatory bowel disease (IBD) in a subject, comprising:
at least one storage device including a set of instructions; and
at least one processor in communication with the at least one storage device, wherein when executing the set of instructions, the at least one processor is directed to perform operations including:
(a) obtaining, from a quantitative measurement device, quantified abundance of one or more target metabolites in a panel of metabolites in a sample from the subject, wherein the panel of metabolites include metabolites of Table 1;
(b) determining a sample score by processing the quantified abundance of each of the one or more target metabolites using a prediction model; and
(c) determining whether the subject has IBD by comparing the sample score to a cut-off score.

2. The system of claim 1, wherein the one or more target metabolites include at least two, three, or four metabolites in Table 1.

3. The system of claim 1, wherein the one or more target metabolites include all the metabolites in Table 1.

4. The system of claim 1, wherein the panel of metabolites further include metabolites in Table 2, wherein the one or more target metabolites include at least one metabolite in Table 1 and at least one metabolite in Table 2.

5. The system of claim 4, wherein the one or more target metabolites include one metabolite in Table 1 and one metabolite in Table 2.

6. The system of claim 4, wherein the one or more target metabolites include one metabolite in Table 1 and two metabolites in Table 2.

7. The system of claim 4, wherein the one or more target metabolites include two metabolites in Table 1 and one metabolite in Table 2.

8. The system of claim 1, wherein the panel of metabolites further include metabolites in Table 3, wherein the one or more target metabolites include at least one metabolite in Table 1 and at least one metabolite in Table 3.

9. The system of claim 1, wherein the panel of metabolites further include metabolites in Table 4, wherein the one or more target metabolites include at least one metabolite in Table 1 and at least one metabolite in Table 4.

10. (canceled)

11. The system of claim 1, wherein the sample score indicates a probability that the subject has IBD.

12-32. (canceled)

33. A system for determining whether a subject has Crohn's disease (CD) or Ulcerative colitis (UC), comprising:
at least one storage device including a set of instructions; and
at least one processor in communication with the at least one storage device, wherein when executing the set of instructions, the at least one processor is directed to perform operations including:
(a) obtaining, from a quantitative measurement device, quantified abundance of one or more target metabolites in a panel of metabolites in a sample from the subject, wherein the panel of metabolites include metabolites of Table 16;
(b) determining a sample score by processing the quantified abundance of each of the one or more target metabolites using a prediction model; and
(c) determining whether the subject has CD or the UC by comparing the sample score to a cut-off score.

34. The system of claim 33, wherein the one or more target metabolites include at least two, three, or four metabolites in Table 16.

35. The system of claim 33, wherein the panel of metabolites further include metabolites in Table 17, wherein the one or more target metabolites include at least one metabolite in Table 16 and at least one metabolite in Table 17.

36. The system of claim 33, wherein the panel of metabolites further metabolites in Table 18, wherein the one or more target metabolites include at least one metabolite in Table 16 and at least one metabolite in Table 18.

37. The system of claim 33, wherein the panel of metabolites further include metabolites in Table 19, wherein the one or more target metabolites include at least one metabolite in Table 16 and at least one metabolite in Table 19.

38-42. (canceled)

43. A system for determining whether a subject has inflammatory bowel disease (IBD) or colorectal polyp, comprising:
at least one storage device including a set of instructions; and
at least one processor in communication with the at least one storage device, wherein when executing the set of instructions, the at least one processor is directed to perform operations including:
(a) obtaining, from a quantitative measurement device, quantified abundance of one or more target metabolites in a panel of metabolites in a sample from the subject, wherein the panel of metabolites include metabolites of Table 21;
(b) determining a sample score by processing the quantified abundance of each of the one or more target metabolites using a prediction model; and
(c) determining whether the subject has IBD or the colorectal polyp by comparing the sample score to a cut-off score.

44. The system of claim 43, wherein the one or more target metabolites include at least two, three, or four metabolites in Table 21.

45. The system of claim 43, wherein the panel of metabolites further include metabolites in Table 22, wherein the one or more target metabolites include at least one metabolite in Table 21 and at least one metabolite in Table 22.

46. The system of claim 43, wherein the panel of metabolites further metabolites in Table 23, wherein the one or more target metabolites include at least one metabolite in Table 21 and at least one metabolite in Table 23.

47. The system of claim 43, wherein the panel of metabolites further include metabolites in Table 24, wherein the one or more target metabolites include at least one metabolite in Table 21 and at least one metabolite in Table 24.

48-91. (canceled)
